(19) United States
(12) Patent Application Publication
(10) Pub. No.: US 2024/0398819 A1
(43) Pub. Date: Dec. 5, 2024

(54) UTILITY OF VITAMIN D, FOLIC ACID AND NADH TO IMPROVE LIGAMENT INJURY PREVENTION CHARACTERISTICS

(71) Applicant: Rhode Island Hospital, Providence, RI (US)

(72) Inventors: Brett D. Owens, Barrington, RI (US); Li Yue, Barrington, RI (US)

(21) Appl. No.: 18/677,766

(22) Filed: May 29, 2024

Related U.S. Application Data
(60) Provisional application No. 63/505,035, filed on May 30, 2023.
(60) Provisional application No. 63/590,993, filed on Oct. 17, 2023.

(51) Int. Cl. A61K 31/525; A61K 31/455; A61K 45/06; A61P 19/04; C12N 5/077

(52) U.S. Cl. CPC A61K 31/525; A61K 31/455; A61K 45/06; A61P 19/04; C12N 5/066

(57) ABSTRACT

Described herein are methods directed to modulating, preventing, and/or treating a connective tissue (e.g., ligament) tissue injury characteristic in a subject. These methods comprise administering vitamin D (or any analogue of) and/or an antagonist of relaxin-2 and/or an antagonist of a receptor of relaxin-2 to the subject, whereby one or more connective tissue characteristics are modulated/changed in the subject. Examples of folic acid and NADH are provided to demonstrate, and data for vitamin D is also demonstrated. Related methods can be utilized for diagnosis of subjects. The methods can be applied to in vitro tissue cultivation in view of lab-cultivated ligaments and connective tissues designed to provide implants for surgery. The methods can be applied to screening of therapeutic agents and to screening of subjects in need of preventative care.

### CROSS-REFERENCE TO RELATED APPLICATIONS

This application claims the benefit of priority to U.S. Provisional Patent No. 63/505,035, filed May 30, 2023, the entire disclosure of which is incorporated by reference as if fully set forth herein in its entirety. This application also claims the benefit of priority to U.S. Provisional Patent No. 63/590,993, filed Oct. 17, 2023, the entire disclosure of which is incorporated by reference as if fully set forth herein in its entirety.

### FIELD OF THE INVENTION

The embodiments of the present invention relate to methods of prevention, diagnosis, and treatment of connective tissue defects/injuries and can be particularly applied to prevention and treatment of acute ligament ruptures/injuries. The methods of prevention and treatment can be directed to timing, delivery, modulation, supplementation, preventative care, and refinement of dosages of various compositions at various locations (e.g., at joints, at ligaments, at tendons) during diagnosis, evaluation, or treatment of a subject in need thereof.

### BACKGROUND OF THE INVENTION

Sports activities may lead to sprains, tears, or ruptures of ligaments and other connective tissue injuries, but the risk of various injuries can vary. For example, more than 120,000 anterior cruciate ligament (ACL) injuries occur annually in the United States (US). Knee ACL reconstruction surgeries are among the most common sports medicine surgeries performed in the US. ACL injuries most commonly occur during active sports, such as soccer, gridiron, skiing, rugby, basketball, and volleyball. Female soccer and basketball athletes have been found to experience roughly three times greater incidence of ACL tears compared to their male counterparts.¹¹² Female athletes are 2 to 8 times more likely to injure their ACL than males.^{66,12} Sex differences (e.g., hormones, anatomic factors, biomechanics) may contribute to the increased incidence of ACL ruptures in females.^{14,52,23} Female ACL injuries alone cost billions of dollars in healthcare costs in the US per year, not to mention quality of life loss for the injured athletes. Recovery from ACL surgery for any person (e.g., females and males) can be painful, can delay/alter/devastate a (sports) career, and can take six months, a year, or longer, typically requiring gradual steps of rehabilitation. Typical prevention techniques for ACL injuries are stretching and balance training. Considering the global population (e.g., young and old) as a whole, new methods to manage connective tissue injuries are urgently needed and focusing on the potential for ACL (and other ligament) injury prevention, new methods to prevent, diagnose, and treat ligament injury characteristics are acutely needed.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Example embodiments of the present invention can provide methods for prevention, diagnosis, and treatment of connective tissue injuries in subjects. In some embodiments, the methods lessen a risk of a connective tissue injury. Ligaments, including the ACL and shoulder ligaments, provide practical, acute, and quickly discernable examples of the invention. Ligament injuries, even after successful acute treatments (i.e., surgery), can predispose a subject to long-term issues surrounding the joint with the treated ligament. In some embodiments, the present innovation contemplates applications for connective tissue management that go beyond acute ligament, neck, shoulder, and ACL injuries and could, for example, be applied to modulation, care, or maintenance of long-term connective tissue injuries/defects, genetic differences (e.g., phenotype), to ankle ligaments, wrist and hand ligaments, and spinal ligaments (e.g., neck or back sprains).

According to an aspect of the present innovation, a method of prevention, conditioning, diagnosis, and/or treatment is provided. The method can include modulating the connective tissue(s) of a subject in need thereof by an administering of an antagonist further described below.

According to an aspect of the present innovation, a formulation with a therapeutic agent is provided, in particular, by a method of designing/locating a therapeutic agent to modulate, prevent a condition in, and/or treat connective tissue(s) in a subject in need thereof. A formulation can be made to include the therapeutic agent. The formulation can be administered or applied by any technique known in the art. A method of designing a therapeutic agent can include screening or consideration for antagonism of relaxin-2 or its receptor(s). In some embodiments, the present innovation provides repurposing for compositions or therapeutic agents by elucidating entirely unexpected treatments for existing compositions or therapeutic agents.

Relaxin-2 is a peptide hormone structurally related to insulin, which is the only known relaxin to circulate in the blood.^{124,137} Relaxin-2 is expressed in the placenta, decidua, prostate, corpus luteum, endometrium, mammary glands, heart, brain,¹⁰ and ovary during pregnancy.⁴⁸ Human relaxin-2 has been used to investigate the effect of testosterone on the expression of relaxin receptor in the knee joint.²⁹ Relaxin-2 enhances the growth of pubic ligaments and ripening of the cervix during birth, has anti-fibrotic and cardioprotective effects,⁹⁷ and has a role in tumor growth and vascularization.⁹³ It may also make female athletes more susceptible to ACL injury.²⁸ A previous study demonstrated that relaxin-2 and estrogen primed cells showed increased expression of matrix metalloproteinases (MMP1 and MMP3) and decreased expression of type I and III collagen, suggesting that relaxin may impact the structural integrity of the ACL, increasing the risk for ACL rupture.⁷⁶

Therefore, the high incidence of female ACL rupture may be due to the changes in relaxin-2 levels. Relaxin-2 activates two orphan G protein coupled receptors: RXFP1, also known as leucine-rich repeat-containing G-protein coupled receptor (LGR)7; and RXFP2, also known as LGR8.⁵⁶ Relaxin receptors have also been found in the dorsoradial ligament, synovium, and articular cartilage of the trapeziometacarpal joint.²² RXFP1 and RXFP2 were found in the ACL tissues, Achilles tendons, inguinal ligaments, and shoulder joint capsules of male wistar rats.⁷⁴ Although relaxin-2 markedly upregulated MMPs and reduced collagen expression in human ligament cells from female but not male ACL tissues,⁷⁶ it is elucidated herein whether this is due to differential expression of RXFP2 between female and male ACL tissues. Herein, we demonstrate how RXFP2 receptors and apoptotic cells are differentially expressed in ACL tissue of male and female patients with ACL reconstruction. There has been very little evidence for the presence of RXFP2 in the male ACL, but consistent evidence of RXFP2 in the female ACL.^{33,39} In a study with the specimens from five females and five males with ACL injures, relaxin binding was present in four out of five female ACL cells versus one out of five male ACL cells.³⁹ Another study found that there was uniform specific relaxin-2 binding in the synovial lining cells, stromal fibroblasts, and cells lining blood vessels of female ACLs but not male ACLs.³³

Apoptosis is a form of programmed cell death, and it plays an important role in proper development and maintenance of tissue homeostasis.¹²⁸ Induction of apoptosis leads to a sequence of characteristic biochemical events, resulting in morphological changes of cells and nuclei and death. Cells undergoing apoptosis display membrane blebbing, DNA fragmentation, cell shrinkage, and degradation of cytoskeletal and nuclear proteins.³⁷ Apoptosis is a normal and controlled process in organisms, but apoptosis reduces tissue healing and the structural integrity of tissues when hyperactive.⁷⁹ Increased cell apoptosis occurs in ACL cells following ACL injuries, evidenced by the activation of caspases, enzymes that regulate and execute apoptosis.^{24,103} After ACL rupture, chondrocyte apoptosis at the ACL tibial insertion was observed for an interval lasting between 19 and 206 days in humans.⁹¹ Cell apoptosis has also been detected in patients with bone tunnel enlargement following ACL reconstruction.¹⁴⁴ However, the relationship between RXFP2 and the apoptosis signaling pathway in human ACL is still under investigation as described herein.

The present innovation, in one of its broadest embodiments, provides a method for preventing and/or treating a connective tissue injury characteristic, the method comprising the steps of: (1) providing a subject in need or in a group characteristic of a risk for injury; and (2) administering an antagonist of relaxin-2 and/or an antagonist of a receptor of relaxin-2 to the subject, whereby one or more connective tissue characteristics are modulated/changed in the subject such as to prevent and/or treat the connective tissue injury characteristic in the subject. In some embodiments, the method above can be carried out by any use of a precursor (or a prodrug) of the antagonist. The subject can be instructed to self-administer an antagonist, for example by utilizing a supplement, a formulation, a natural product, or a dietary change. The method disclosed herein can be, in some embodiments, wherein the connective tissue comprises a ligament, and the antagonist comprises folic acid, NADH, or a combination thereof.

In some embodiments, the method disclosed above can be wherein the subject has previously undergone a surgical reconstruction, and/or can be a normal healthy subject, lacking a pre-diagnosed connective tissue disease or condition, and step (2) comprises in part a prophylactic method used to prevent a connective tissue injury/rupture. In some embodiments, the methods disclosed above can be wherein the subject is expected to or likely to participate in an activity, a sport, or a movement in the future that can increase the chances of a connective tissue injury occurring in the subject.

According to some aspects, the method disclosed above can be wherein the subject is at a risk of or in need of a connective tissue care, having a diagnosed connective tissue injury characteristic, or has been determined to be at a risk of a connective tissue injury by a health care provider. According to some aspects, the method disclosed above is wherein the subject is suspected of having or has been diagnosed by a health care provider as having, a disorder including an autoimmune disease of connective tissue, an undifferentiated connective tissue disorder, a myxomatous degeneration, a congenital disease, a neoplasm, or a combination thereof.

In some embodiments, a method for cultivating a connective tissue in vitro is provided, the method comprising the step of contacting the cultured connective tissue with an antagonist of relaxin-2 and/or an antagonist of a receptor of relaxin-2, before, during, or after said cultivating, whereby one or more connective tissue characteristics are modulated/changed. In some embodiments, the method of cultivating can be wherein the connective tissue is suitable for implantation into a subject in need thereof. In some embodiments, this method can be wherein the connective tissue is a cell generated tissue graft and/or wherein the connective tissue cultivated in vitro is derived from or originates from a subject undergoing, or planned to undergo, a ligament reconstruction surgery.

The method of any preceding paragraph, in some embodiments, can be wherein the connective tissue includes ligament, tendon, cartilage, intervertebral disc, cornea, or a combination thereof. The connective tissue can comprise a fibrillar collagen or a collagen. For example, the collagen can include type I (or IA), II, III (or IIIA), V, XI, or a combination thereof.

The methods disclosed above, in some embodiments, can be wherein the antagonist comprises a therapeutic agent, a supplement, a formulation, a natural product, a drug, a biologic, an aerosol, a liquid, a particle, microparticle, or nanoparticle, a biologic, or a combination thereof. In an example, the antagonist includes a daily dose of about 400 micrograms/day antagonist for a healthy human subject, based on general daily recommended values. In some embodiments, a daily dose can be about less than 1000 mg per day, less than about 500 mg per day, less than about 250 mg per day, less than about 100 mg per day, less than about 10 mg per day, less than about 1 mg per day, or less than about 500 micrograms per day.

In some embodiments, the methods disclosed above can be wherein the method is long-term. The antagonists can be, for example, a blocker, an orthosteric antagonist, allosteric antagonist, conformation changing binder, a modulator, a partial inverse agonist/inverse agonist, or a combination thereof.

The method of any preceding paragraph above, in some embodiments, can be wherein the method is in the form of instructions provided in a kit, said kit optionally including a formulation and/or therapeutic agent operative to block/antagonize relaxin-2 and/or a block/antagonize a receptor of relaxin-2.

In some embodiments, a method for diagnosing and/or determining a risk or a likelihood of a connective tissue injury/defect in a subject is disclosed herein, the method comprising quantifying the number of RXFP2 positive cells in a connective tissue derived from the subject and comparing the number to RXFP2 positive cells to a standard determination.

A method for designing, screening, and/or locating a therapeutic agent, said therapeutic agent suitable for preventing and/or treating a connective tissue injury characteristic in a subject in need thereof is demonstrated herein, the method comprising the steps of: (1) providing a candidate therapeutic agent suspected of having activity including antagonism of relaxin-2 and/or antagonism of a receptor of relaxin-2; and (2) screening the candidate therapeutic agent such as to determine/measure antagonism of relaxin-2 and/or a receptor of relaxin-2.

The screening method can be wherein the method is repeated for a plurality of candidate therapeutic agents and a measurement/determination of antagonism of relaxin-2 and/or a receptor of relaxin-2 is provided for each of the candidate therapeutic agents. Each of the candidate therapeutic agents can be ranked in comparison to the other each of the candidate therapeutic agents for an efficacy of relaxin-2 and/or a receptor of relaxin-2.

The methods described above can, in some embodiments, include wherein an expression/activity of MMP1 and/or MMP13 is decreased by the method.

The above-described methods can include administering and/or applying a therapeutic agent to a subject. The therapeutic agent can have a potency in any range suitable to provide an effect in a subject in need thereof. The effect can be immediate and readily discernable, for example, by a decrease in injuries. The effect can be a long-term effect that requires years and/or a statistical significance to discern. The therapeutic agent can be administered in combination with any other drug or therapeutic agent and can have efficacy in the prevention, modulation, treatment, and/or diagnosis of connective tissue(s') injuries. The present invention can be utilized to design therapeutic agents to perform the methods disclosed herein. The therapeutic agents can be delivered alone or can be linked to other agents (e.g., antibody conjugates, liposome formulations, inhaled particles, time-released formulations, micro/nanoparticles). In some embodiments, a targeting moiety can be utilized with the therapeutic agents. In some embodiments, the targeting moiety is an antibody with affinity for a specific type of cell (e.g., a specific type of connective tissue). In another example, the targeting moiety is a nanoparticle. In some embodiments, a formulation or a therapeutic agent can be administered directly to a tissue in need thereof, for example, when an acute injury is treated, by use of a topical (skin penetrating) formulation), or in an emergency. The methods disclosed herein can be utilized to diagnose subjects' risk of being in need of connective tissue treatment. The methods can be applied to populations, for example, with machine learning, or can be applied to individuals, for example, using personalized medicine.

As an additional brief summary, some features of the technology disclosed herein can be briefly summarized by the following list of features:

Feature 1: A method for preventing and/or treating a connective tissue injury characteristic, the method comprising the steps of: (1) providing a subject; and (2) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury.

Feature 2: The method of feature 1, wherein the connective tissue comprises a ligament, and the antagonist comprises folic acid, NADH, or a combination thereof.

Feature 3: The method of any preceding feature, wherein the subject is a subject that has previously undergone a surgical reconstruction, and/or a normal healthy subject, lacking a pre-diagnosed connective tissue disease or condition, and step (2) comprises in part a prophylactic method used to prevent a connective tissue injury and/or rupture.

Feature 4: The method of any preceding feature, wherein the subject is expected to or likely to participate in an activity, a sport, or a movement in the future that can increase the chances of a connective tissue injury occurring in the subject.

Feature 5: The method of feature 1, wherein the subject is at a risk of or in need of a connective tissue care, having a diagnosed connective tissue injury characteristic, or has been determined to be at a risk of a connective tissue injury by a health care provider.

Feature 6: The method of feature 1, wherein the subject is suspected of having, or has been diagnosed by a health care provider as having, a disorder including an autoimmune disease of connective tissue, an undifferentiated connective tissue disorder, a myxomatous degeneration, a congenital disease, a neoplasm, or a combination thereof.

Feature 7: A method for cultivating a connective tissue in vitro, the method comprising the step of contacting the culturing connective tissue with an antagonist of relaxin-2 and/or an antagonist of a receptor of relaxin-2, or a precursor of said antagonist, before, during, or after said cultivating, whereby one or more connective tissue characteristics are modulated/changed.

Feature 8: The method of feature 7, wherein the connective tissue is suitable for implantation into a subject in need thereof.

Feature 9: The method of feature 7, wherein the connective tissue is a cell generated tissue graft.

Feature 10: The method of feature 7, wherein the connective tissue cultivated in vitro is derived from or originates from a subject undergoing, or planned to undergo, a ligament reconstruction surgery.

Feature 11: The method of any preceding feature, wherein the connective tissue includes ligament, tendon, cartilage, intervertebral disc, cornea, or a combination thereof.

Feature 12: The method of feature 11, wherein the connective tissue comprises a ligament.

Feature 13: The method of any preceding feature, wherein the connective tissue comprises a fibrillar collagen or a collagen.

Feature 14: The method of feature 13, wherein the collagen comprises type I (or IA), II, III (or IIIA), V, XI, or a combination thereof.

Feature 15: The method of any preceding feature, wherein the antagonist comprises a therapeutic agent, a supplement, a formulation, a natural product, a drug, a biologic, an aerosol, a liquid, a particle, microparticle, or nanoparticle, a biologic, or a combination thereof.

Feature 16: The method of feature 15, wherein the antagonist includes a daily dose of about 400 micrograms/day antagonist for a healthy human subject.

Feature 17: The method of any preceding feature, wherein the method is long-term.

Feature 18: The method of any preceding feature, wherein the antagonist is a blocker, an orthosteric antagonist, allosteric antagonist, conformation changing binder, a modulator, a partial inverse agonist/inverse agonist, or a combination thereof.

Feature 19: The method of any preceding feature, wherein the method is in the form of instructions provided in a kit, said kit optionally including a formulation and/or therapeutic agent operative to block/antagonize relaxin-2 and/or a block/antagonize a receptor of relaxin-2.

Feature 20: The method of feature 18, wherein the antagonist comprises a relaxin antagonist/relaxin receptor antagonist from examples in Table 2 and/or Table 3, or from a library of known therapeutic agents.

Feature 21: A method for diagnosing and/or determining a risk or a likelihood of a connective tissue injury/defect in a subject, the method comprising quantifying the number of RXFP2 positive cells in a connective tissue derived from the subject and comparing the number to RXFP2 positive cells to a standard determination.

Feature 22: A method for designing, screening, and/or locating a therapeutic agent, said therapeutic agent suitable for preventing and/or treating a connective tissue injury characteristic in a subject in need thereof, the method comprising the steps of: (1) providing a candidate therapeutic agent, or a precursor of the candidate, suspected of having activity including antagonism of relaxin-2 and/or antagonism of a receptor of relaxin-2; and (2) screening the candidate therapeutic agent or precursor such as to determine/measure antagonism of relaxin-2 and/or a receptor of relaxin-2, wherein said antagonism is determinative of the suitability for prevention of an injury.

Feature 23: The method of feature 22, wherein the method is repeated for a plurality of candidate therapeutic agents and a measurement/determination of antagonism of relaxin-2 and/or a receptor of relaxin-2 is provided for each of the candidate therapeutic agents.

Feature 24: The method of feature 23, wherein each of the candidate therapeutic agents is ranked in comparison to the other each of the candidate therapeutic agents for an efficacy of relaxin-2 and/or a receptor of relaxin-2.

Feature 25: A method for preventing and/or treating a connective tissue injury characteristic, the method comprising the steps of: (1) providing a subject; and (2) administering vitamin D (D1, D2, D3, D4, D5, or an analogue thereof) to the subject, or a precursor of said vitamin D, whereby one or more connective tissue characteristics are modulated/changed in the subject such as to prevent and/or treat the connective tissue injury.

Feature 26: A method for cultivating a connective tissue in vitro, the method comprising the step of contacting the culturing connective tissue with vitamin D (D1, D2, D3, D4, D5, or an analogue thereof), or a precursor of said vitamin D, whereby: (1) one or more connective tissue characteristics are modulated/changed; and (2) one or more differences in effects of the modulated/changed are observed between cells derived from a female subject and cells derived from a non-female or a male subject.

Feature 27: The method of feature 25 or feature 26, wherein the vitamin D comprises vitamin D3 (cholecalciferol), vitamin D2 (ergocalciferol), or a combination thereof.

Feature 28: The method of feature 25 or 26, wherein the analogue comprises Alfacalcidol, Calcipotriol (calcipotriene), Doxercalciferol, Falecalcitriol, Paricalcitol, Tacalcitol, or a combination thereof.

Feature 29: The method of feature 25 or 26, wherein the method is used in any combination with the method of any one of features 1-24, or before or after any one of features 1-24. Any of the above features can be combined with an administration of vitamin D.

Feature 30: The method of any one of features 25-29, wherein Type I collagen expression is observably increased after vitamin D in female cells compared to such observable expression under the same conditions in male cells.

Feature 31: The method of any preceding feature, wherein an expression/activity of MMP1 and/or MMP13 is decreased by the method.

Feature 32: The method of feature 31, wherein MMP13 gene expression is different between cells derived from a female subject and cells derived from a male subject after one or more vitamin D treatments.

Feature 33: The method of feature 31, wherein MMIP13 gene expression is reduced after one or more vitamin D treatments at about 10 nmol/L in female cells and at about 100 nmol/L in male cells.

Feature 34: A method for preventing and/or treating a connective tissue injury characteristic, the method comprising the steps of: (1) providing a subject; (2) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury; and (3) administering vitamin D (D1, D2, D3, D4, D5, or an analogue thereof) to the subject, or a precursor of said vitamin D, whereby one or more connective tissue characteristics are modulated/changed in the subject such as to prevent and/or treat the connective tissue injury; wherein an execution of steps (2) and (3) is separated by a time period of less than about 1 year.

While the summary examples disclosed above provide some introduction to embodiments of the invention, other implementations are also contemplated, described, and recited herein. These and other features and advantages will be apparent from a reading of the following detailed description, the Examples, and a review of the associated drawings. It is to be understood that both the foregoing general description and the following detailed description are explanatory only and are not restrictive of aspects as claimed.

### BRIEF DESCRIPTION OF THE DRAWINGS

For the purpose of illustration, certain discernable embodiments of the present invention are shown in the drawings described below. It should be understood, however, that the invention is not limited to the precise arrangements, data, dimensions, and illustrations shown. In the drawings:

FIGS. 1A-1F provide RXFP2 expression in studied patients' ACL tissues. FIG. 1A provides the age of male patients (n=10) and female patients (n=10) with ACL reconstruction. FIG. 1B shows representative images of negative and positive RXFP2 immunostaining in ACLs of healthy donor and patients with ACL reconstruction. Scale bar=50 μm. In FIG. 1B, lighter gray arrows (previously shown as red in color) indicate RXFP2 positive immunostaining (i.e., brown to dark brown color). Darker black arrows indicate RXFP2 negative immunostaining (i.e., blue/purple color). FIG. 1C shows the number of RXFP2 positive cells in 1 mm² on ACLs of normal control donors, male and female patients. Correlation between relaxin-2 receptor expression and patient's age is provided in male patients (FIG. 1D), female patients (FIG. 1E), and all 20 patients (FIG. 1F). In FIGS. 1A-1F, *p<0.05 vs. normal control group.

FIGS. 2A-2E provide ACL cell apoptosis in patient ACL tissues. FIG. 2A shows representative images of negative and apoptotic staining in ACLs of healthy donor and patients with ACL reconstruction. Scale bar=50 μm. Lighter gray (previously shown as red in color) arrows indicate apoptotic positive immunostaining (i.e., brown precipitate). Darker black arrows indicate negative apoptotic immunostaining (i.e., green color). FIG. 2B shows the number of apoptotic positive cells in 1 mm² on ACLs of male and female patients. Correlation is shown between the number of apoptotic cells and age of male patients (FIG. 2C) or female patients (FIG. 2D), and all 20 patients (FIG. 2E). *p<0.05 and **p<0.01 vs. normal control group.

FIGS. 3A-3G provide correlations between RXFP expression and cell apoptosis, and cAMP expression in the ACL tissues. Correlations are shown between RXFP expression and cell apoptosis of male patients (FIG. 3A), female patients (FIG. 3B), and all 20 patients (FIG. 3C).

FIG. 3D provides cAMP concentration (pmol/mg) on ACL of 8 male patients and 9 female patients. Correlation is shown between cAMP concentration and age in male patients (FIG. 3E) and in all patients (FIG. 3F). FIG. 3G shows correlation between the number of apoptotic cells and time from injury (months).

FIGS. 4A-4C provide expressions of MMP1 and MMP13 in patient ACL tissues. FIG. 4A shows progressive morphologic changes of patient ACL cells. Scale bar=100 μm. Expression of MMP1 (FIG. 4B) and MMP13 (FIG. 4C) in 8 male and 9 female patient ACL cells is provided. Data were examined by one-way analysis of variance (ANOVA) with Tukey post-hoc multiple comparison, *p<0.05.

FIGS. 5A-5E provide expressions of MMP13, collagen 1A, collagen 3A, MMP3, and MMP1 in patient ACL tissues. The figures show plots of (FIG. 5A) MMP13 expression; (FIG. 5B) collagen 1A expression; (FIG. 5C) collagen 3A expression; (FIG. 5D) MMP3 expression; and (FIG. 5E) MMP1 expression. In FIGS. 5A-5E, the protocol for patient enrollment was approved by the IRB (Institutional Review Board) committee at Rhode Island Hospital. GraphPad PRISM 8 software was used to perform all statistical analyses. Results were presented as mean+standard error of the mean (SEM) of different samples and considered significant when p<0.05. A t-test was used to compare any two groups, *p<0.05, **p<0.01, ***p<0.001 compared to their individual control group; ##p<0.01, compared to their individual relaxin-2 group.

FIGS. 6A-6C provide expression of RXFP2 on shoulder capsules and labra from 20 patients with shoulder instability. Immunohistochemical staining of a relaxin-2 receptor RXFP2 was performed in patient shoulder specimens. The brown precipitate (seen as gray in grayscale, FIG. 6A, FIG. 6B) indicates the presence of the target antigen RXFP2. The nucleus is stained purple (slightly darker gray) by hematoxylin staining. Combination of purple and brown colors resulted in dark brown (darker gray). The brown and dark brown cells were counted as RXFP2 positive cells. FIG. 6A shows a representative image of a negative control and a positive control of immunohistochemistry. Scale bar (lower left)=50 μm. Negative control in human labrum tissues was not incubated with a RXFP2 antibody. RXFP2 immunohistochemistry in shoulder labrum of male Wistar rat was used as a positive control. FIG. 6B shows representative immunohistochemistry images of RXFP2 positive cells in capsule and labrum tissues of patients with shoulder instability. The gray arrows (previously red in color) indicate RXFP2 positive cells (brown/gray to dark brown/gray precipitate). FIG. 6C shows quantification of the number of RXFP2 positive cells in 1 mm² field from male patients (n=15) and female patients (n=5). Results were presented as mean±SD of different samples, as the dots are absolute number of RXFP2 positive cells. **P<0.01 vs male.

FIGS. 7A-7B provide expression of RXFP2 in vessels and nucleus in shoulder specimens from patients with shoulder instability. Immunohistochemical staining of a relaxin-2 receptor RXFP2 were performed in patient shoulder specimens. The brown (gray) precipitate indicates the presence of the target antigen RXFP2. The nucleus is stained purple (darker gray) by hematoxylin staining. Combination of purple and brown colors resulted in dark brown/gray. The brown and dark brown cells were counted as RXFP2 positive cells. Representative immunohistochemistry images of RXFP2 located in vessels (FIG. 7A) and nucleus (FIG. 7B) of capsules and labra in patients with shoulder instability. Gray arrows indicate RXFP2 positive immunostaining (brown to dark brown precipitate). Black arrows indicate RXFP2 negative immunostaining (previously purple color).

FIGS. 8A-8G provide data supporting no correlation between RXFP2 expression and ages in 20 patients with shoulder instability. Immunohistochemical staining of a relaxin-2 receptor RXFP2 were performed in shoulder capsules and labra from 20 patients with shoulder instability. The number of RXFP2 positive cells in 1 mm² field were counted and the correlation between age and RXFP2 number in the male and female patients were calculated. FIG. 8A is a plot of the age of male (n=15) and female (n=5) patients with shoulder instability that was present. The correlation between age and RXFP2 number in 1 mm² field on capsules (FIG. 8B) of patients including twelve male (FIG. 8C) and five female (FIG. 8D) patients. The correlation between age and RXFP2 number in 1 mm² field on labra (FIG. 8E) of patients, including fifteen male (FIG. 8F) and five female (FIG. 8G) patients. The dots (symbolized on the plots) are absolute number of RXFP2 positive cells for each group.

FIG. 9 provides a flow diagram depicting patient selection and number of exclusions per exclusion criteria for the analysis of index anterior cruciate ligament injuries.

FIG. 10 shows a flow diagram depicting patient selection and number of exclusions per exclusion criteria for the analysis of anterior cruciate ligament reconstruction failure with revision reconstruction.

FIG. 11 provides a bar chart of incidence of anterior cruciate ligament tears in patients with vitamin D deficiency; and a comparison with a matched cohort; 95% confidence intervals are reported as error bars.

FIG. 12 shows a table of rates of anterior cruciate ligament (ACL) tears within 1 and 2 years of a diagnosis of vitamin D deficiency. A comparison among overall and sex- and age-specific subgroups.

FIG. 13 is a table showing number and percentage of ACL tears that underwent surgical reconstruction within two years of the index ACL tear.

FIG. 14 provides a table showing rates of revision anterior cruciate ligament (ACL) reconstruction or repair within 1 and 2 years of a primary ACL reconstruction or repair in patients with and without a recent diagnosis of vitamin D deficiency. A comparison among overall and sex- and age-specific subgroups.

It should be understood that while different illustrations are sometimes used in some of the figures above to describe different embodiments and different aspects of the technology, any aspect from any figure can be inter-combined with an aspect from any other figures. All trademarks, images, likenesses, words, and depictions in the drawings and the disclosure are plainly in fair use and are provided solely for the purposes of illustration of the invention in view of an urgent need to prevent injuries and to treat subjects as further discussed in more detail below.

### DETAILED DESCRIPTION OF THE INVENTION

The subject innovation is now described, in some examples with reference to the drawings, wherein examples can used to refer to the aspects of the breadth of concepts of the invention. In the following description, for purposes of explanation, specific details are set forth in order to provide a thorough understanding of the present invention. It may be evident, however, that the present invention may be practiced without these specific details. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention can be determined by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “range” may be provided. A statement may include “in the range from about A to about B”. All points from A to B are subsumed by the range, and all those points can define preferred ranges. Within said range, any range subsumed therein means any range that is within the stated range. Endpoints within the range can define a new range. For example, the following are all subsumed within the range of about 10 to about 50. 10 to 20; 15 to 35; 23 to 40; or 50 to 31; or any other range or set of ranges within the stated range. As such, within the range any set of endpoints subsumed therein can be used as an exemplary range.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. In specific examples, “consisting essentially of” can be explained herein for each example or can be defined broadly, for example, by stating that an administration to a subject (in a method herein) does not include any other active pharmaceutical ingredient or therapeutic agent in addition to the one specified.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), racing mammals, laboratory animals (e.g., mice, rats, rabbits, etc.), but are not so limited. Subjects include human subjects. The human subject may be a pediatric, adult, or a geriatric subject. The human subject may be of either sex. In another example, the term “subject” can refer to a connective tissue culture, and the methods disclosed herein, while claimed towards subjects, contemplate use in the laboratory in synthetic tissue(s). As used herein, a female cell can refer to a cell with 2X chromosomes; a male cell can refer to a cell with 1X and 1Y chromosome.

As used herein, the terms “effective amount” and “therapeutically effective amount” include an amount sufficient to modulate a treatment or prevent or ameliorate a manifestation of disease or medical condition, such as a connective tissue condition or a risk of a connective tissue injury. Such a condition (or risk) may not be readily discernable and may take years, statistical analysis, and/or machine learning to determine a prevention, treatment, or amelioration. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the condition and on the characteristics of the individual, such as general health, age, sex, body weight and tolerance to drugs. It will also depend on the degree, severity and type of condition. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, manage, modulate, slow down or stop the progression or severity of a symptom or condition. The term “treating” includes reducing or alleviating at least one adverse effect (undesirable characteristic) or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a condition or decay, delay or slowing of a progression and/or risk of injury, and an increased lifespan/enjoyment as compared to that expected in the absence of treatment.

As used herein, the term “long-term” administration means that the therapeutic agent or drug is administered for a period of at least 12 weeks. The therapeutic agent or drug may refer to a formulation, composition, or agent. The formulation can be changed to a fresh formulation during administration. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by any technique known in the art that is subsequently applied to a subject, topical application, absorption, injection, ingestion, transfusion, implantation or transplantation. In an example embodiment, compositions are applied as a tablet or drug in capsule. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subdermal, subcuticular, intraarticular, subcapsular, subarachnoid, intraspinal and intrasternal injection and infusion. It is known in the art that therapeutic agents can be rapidly deployed through the skin and directly into joint/ligaments by use of DMSO (dimethyl sulfoxide) as a carrier solvent applied (with the therapeutic agent) to the skin near to or surrounding a joint. While DMSO is rarely used anymore for these purposes because of its nature as a universal solvent and its tendency to carry any residual chemicals present on the skin into the bloodstream (along with the intended agent), the technology contemplates such uses. In one contemplated embodiment, the compositions contemplated herein are administered to a subject by direct injection into a tissue, lymph node, or site of treatment. In another example, administration is provided in the form of a natural product, vitamin, supplement, food, aerosol, inhalation, vapor, or drink. Formulations disclosed herein can be ready made or require mixing just before administration.

Any of the methods disclosed herein can be carried out in part or completely by including a dietary change, a food, natural product, precursor, or prodrug of a therapeutic agent. As used herein, a precursor or a prodrug is intended to encompass compounds or therapeutic agents which, under physiologic conditions, are converted into the therapeutically active agents of the present invention (e.g., a compound for any of the present claims or features). A common method for making a prodrug is to include one or more selected moieties which are hydrolyzed under physiologic conditions to reveal the desired molecule. In other embodiments, the prodrug is converted by an enzymatic activity of the host subject. For example, esters or carbonates (e.g., esters or carbonates of alcohols or carboxylic acids) are preferred prodrugs of the present invention. In certain embodiments, some or all of the compounds selected from Table 2 or 3 in a formulation represented above can be replaced with the corresponding suitable prodrug, for example, wherein a hydroxyl in the parent compound is presented as an ester or a carbonate or carboxylic acid present in the parent compound is presented as an ester. A common method of making a precursor/prodrug that can be used herein is to use a carrier/nanocarrier (e.g., mesoporous silica particles). The precursor/prodrug can be released from a carrier to form the active therapeutic agent. A precursor or prodrug can be metabolized to the active parent compound (therapeutic agent) in vivo (e.g., the ester is hydrolyzed to the corresponding hydroxyl, or carboxylic acid).

The terms. “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms: “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term: “small molecule” refers to a molecule that has a molecular weight <1000. As used herein, the term: “large molecule” refers to a molecule that has a molecular weight >1000, and the term includes biologics such as the examples of oligonucleotides, peptides, antibodies, linkers, oligosaccharides, polymers, DNA chains, and RNA chains. The term: “therapeutic agent” may refer to small molecule, element, large molecule, biologic, formulation, composition, agent, or a combination thereof.

### Relaxin-2 and its Receptor Antagonists to Improve Ligament Injury Prevention Characteristics:

Disclosed herein is the first study on quantification of relaxin-2 receptor RXFP2/LGR8 number in male and female patients with ACL reconstruction as compared to normal ACL tissues. This study openly displays the connection between ACL injury and relaxin-2 receptors in both male and female patients. We found relaxin-2 receptor expression was higher in patients with ACL reconstruction.

Peptide hormone relaxin-2, a member of the insulin family, enhances the growth of pubic ligaments and ripening of the cervix during birth as well as increases the risk for ACL ruptures. Serum relaxin levels are similar between young athletic men and women. We found that apoptotic cells are present in paraffin embedded specimens from patients with bone tunnel enlargement after ACL reconstruction. Relaxin receptors are present at the ligament, cartilage, and synovium of the trapeziometacarpal joints from female and male patients. Although relaxin-2 markedly upregulated matrix metalloproteinase (MIMP) and reduced collagen expression in human ligament cells from female but not male ACL tissues, it is elucidated herein whether this is due to differentially expression of relaxin-2 peptide receptor 2 (RXFP2) between female and male ACL tissues. It is unknown the correlation between apoptosis and RXFP2 expression. We demonstrate herein the extent that RXFP2 is differentially expressed in ACL tissues between male and female ACLs.

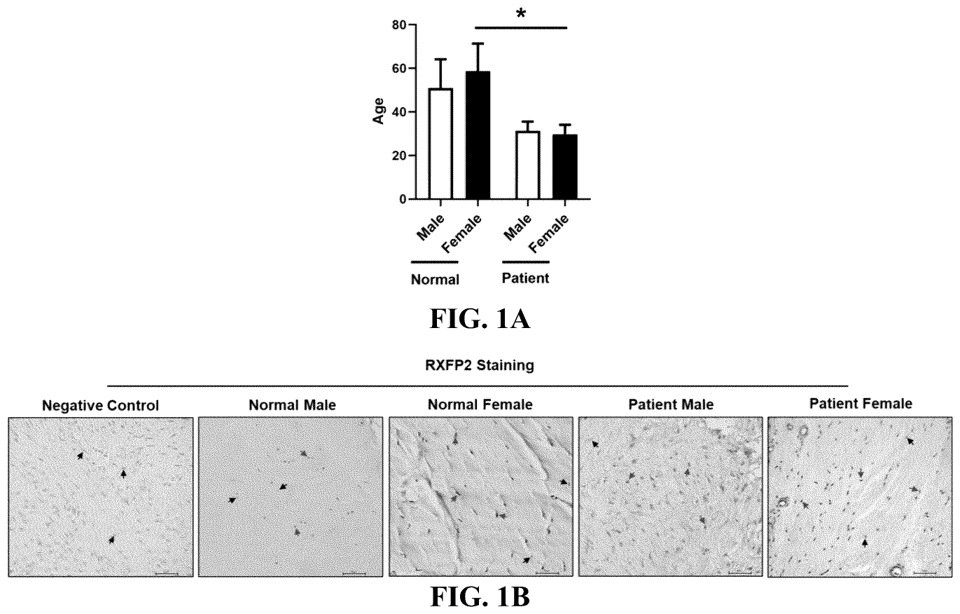

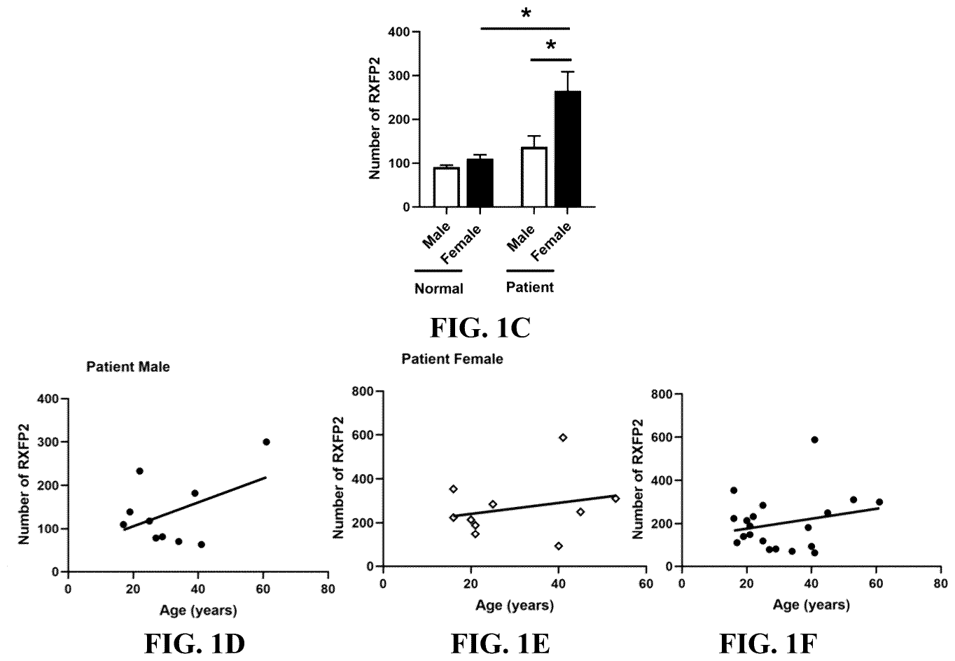

In an initial study, ACL samples of 20 patients (10 males and 10 females), 4 normal males and 4 normal females are collected. Examples 1-3 below describe further details. Patient demographics and clinical characteristic are described in Table 1 below. The mean age of all patients was 30.6 years old, with patient ages ranging from 16 years to 61 years old. The mean age of 10 male patients was 31.4±4.15, and the mean age of 10 female patients was 29.8±4.29. The mean age of 4 normal male donors was 51±13.18, and the mean age of 4 normal female donors was 58.75±12.63, with donors' age ranging from 24 years to 84 years old (FIG. 1A). There was no discernible difference in age between male and female patients. The age of normal female donors was significantly higher than that of female patients.

TABLE 1
Patient Demographics and Clinical Characteristics.

| | | | | | Time to | |
|---|---|---|---|---|---|---|
| | Date of | | | | surgery in | Oral Contraceptive |
| Patient # | Surgery | Age, y | Gender | Race | months | use |
| 1 | Jan. 7, 2020 | 20 | Female | White | 1 | y |
| 2 | Jan. 7, 2020 | 27 | Male | Black | 2.5 | n/a |
| 3 | Jan. 14, 2020 | 61 | Male | White | 6 | n/a |
| 4 | Jan. 28, 2020 | 16 | Female | White | 1 | n |
| 5 | Feb. 4, 2020 | 21 | Female | Asian | 24 | y |
| 6 | Feb. 4, 2020 | 41 | Female | White | 1 | y |
| 7 | Feb. 5, 2020 | 25 | Female | White | 1.5 | n |
| 8 | Feb. 12, 2020 | 22 | Male | White | 2.5 | n/a |
| 9 | Feb. 26, 2020 | 41 | Male | White | 1 | n/a |
| 10 | Feb. 26, 2020 | 40 | Female | White | 0.5 | n |
| 11 | Mar. 3, 2020 | 53 | Female | White | 5 | n but on |
| | | | | | | progesterone med for |
| | | | | | | menopause |
| 12 | Sep. 1, 2020 | 21 | Female | White | 24 | n |
| 13 | Sep. 15, 2020 | 39 | Male | White | 6 | n/a |
| 14 | Oct. 27, 2020 | 45 | Female | White | 1.5 | n |
| 15 | Nov. 3, 2020 | 16 | Female | White | 0.5 | y |
| 16 | Nov. 17, 2020 | 17 | Male | White | 11 | n/a |
| 17 | Nov. 18, 2020 | 19 | Male | White | 1 | n/a |
| 18 | Dec. 2, 2020 | 25 | Male | White | 40 | n/a |
| 19 | Dec. 4, 2020 | 29 | Male | Hispanic | 2 | n/a |
| 20 | Dec. 8, 2020 | 34 | Male | White | 2 | n/a |

### RXFP2 Expression in the ACL Tissues

Immunohistochemical staining of a relaxin-2 receptor RXFP2 was performed on ACL samples from all 20 male and female patient samples. Nuclei were indicated by blue/purple (shown as darker gray in grayscale) hematoxylin staining (FIG. 1B). This served as a negative control. The presence of the target antigen RFXP2 was indicated by the presence of a brown or dark brown (darker gray) precipitate (FIG. 1B). The dark brown precipitate resulted when the blue/purple stained nuclei were combined with the RXFP2 brown staining. We found that both male and female ACL samples of patients and normal donors expressed RXFP2. The number of RXFP2 positive cells was found to be markedly higher in ACL tissues from female patients as compared to that of male patients or normal female donors. There was no difference on the number of RXFP2 positive cells among normal male donors, normal female donors and male patients. The mean RXFP2 numbers of human ACLs were as follows: Normal male donor was 91.5±3.97, normal female donor was 110.8±8.38, male patient was 137.4±24.83, and female patient was 265.6±43.25 (FIG. 1C). These results suggest that the activity of relaxin-2 may be related to gender and may be more upregulated in female ACL tissues than that of males.

Data were examined using Pearson's correlation test. The correlation coefficient r was 0.4595 (r²=0.2112, p=0.1815) for males in FIG. 1D, r was 0.2483 (r²=0.06165, p=0.4891) for female patients in FIG. 1E, and r was 0.22352 (r²=0.05531, p=0.3182) for all patients in FIG. 1F. These results indicate that a weak relationship exists between RXFP2 expression and age in ACLs from these 20 patients, and in female patients. There was a significant correlation between RXFP2 expression and age in male patients.

### Apoptotic Expression in the ACL Tissues

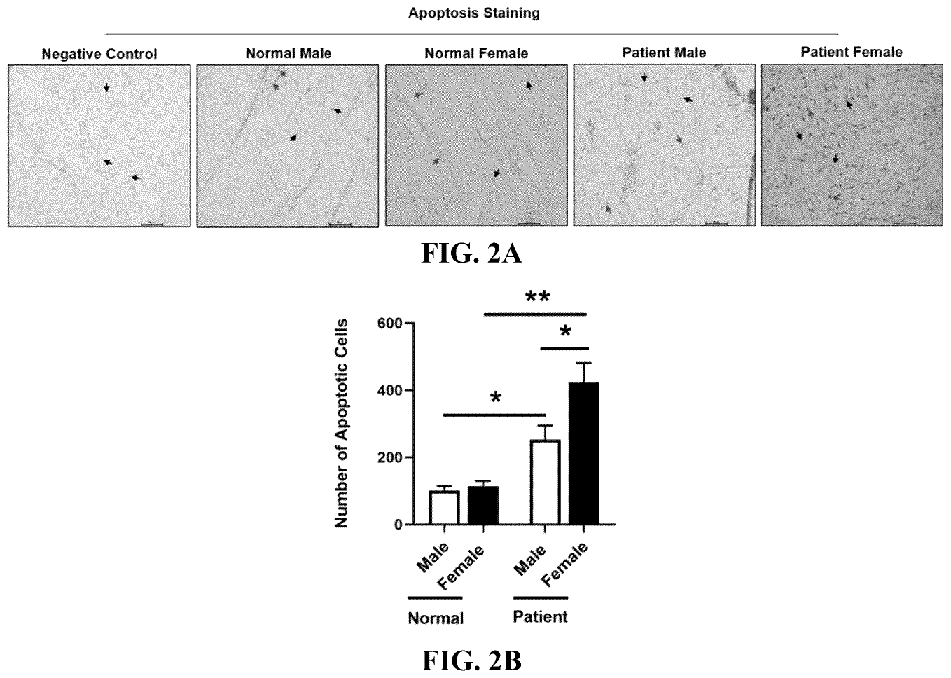

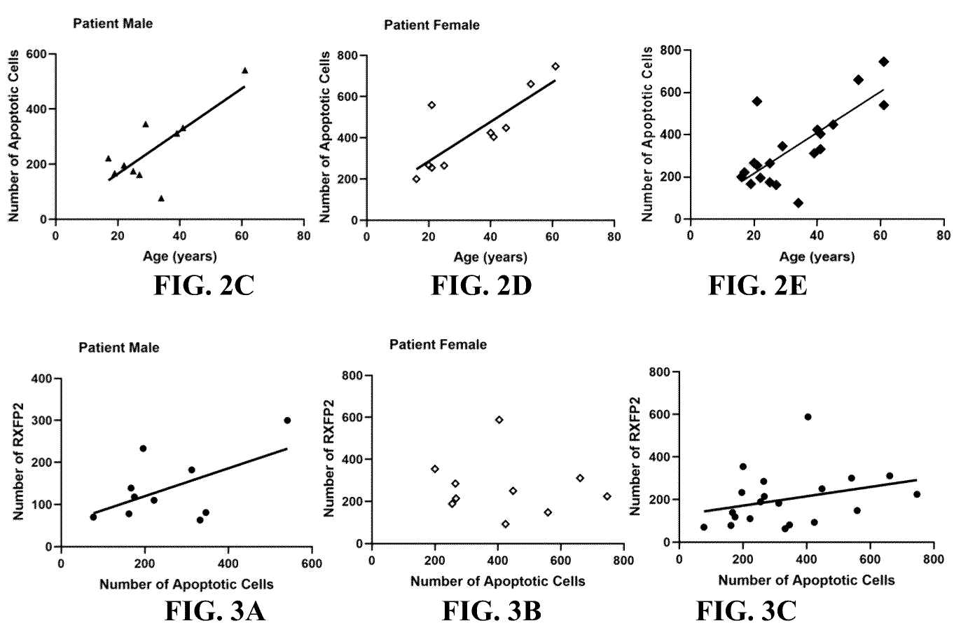

Both male and female ACL samples of patients and normal donors expressed apoptosis. Apoptotic expression was found to be significantly higher in female ACL tissues compared to that of male patients or normal female donors. The number of apoptotic cells in male patient's ACLs was markedly higher than that of normal male donors. There was no difference on the number of apoptotic cells between normal male donors and normal female donors. The mean apoptotic cell number of normal male donor was 101±13.04, normal female donor was 114±15.74, 10 male patients was 253±41.93, and 10 female patients was 422.7±58.49 (FIG. 2A and FIG. 2B). These results suggest that increased apoptotic activity may be related to gender and may be more upregulated in female ACL tissues than that of males after ACL injury. Correlation coefficient r was 0.7665 (r²=0.5875, p=0.0097) for males in FIG. 2C, r was 0.8174 (r²=0.6681, p=0.0039) for females in FIG. 2D, r was 0.7196 (r²=0.5178, p<0.0001) for all patients in FIG. 2E. These results indicate that a strong positive correlation was found between apoptotic cells and age of male patients, between apoptotic cells and female patients, between apoptotic cells and all 20 patients.

### Correlations Between RXFP2 Expression and Apoptotic Cell Number

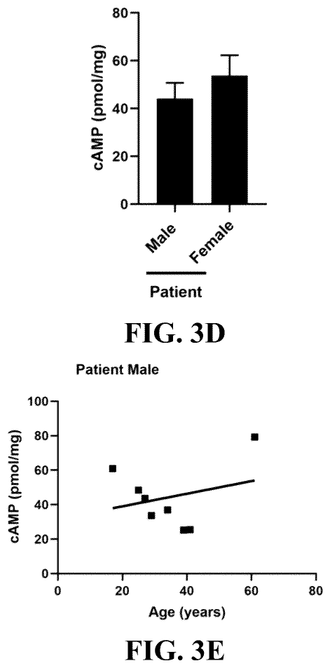

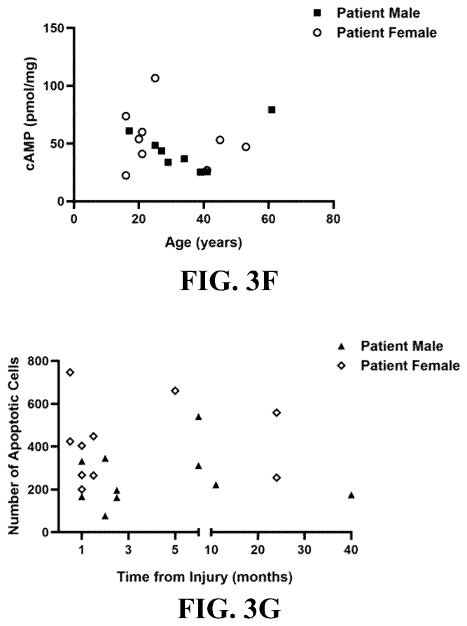

Correlation coefficient r was 0.5590 (r²=0.3125, p=0.0930) for males in FIG. 3A, r was −0.1195 (r²=0.01427, p=0.7424) for females in FIG. 3B, r was 0.3102 (r²=0.09622, p=0.1832) for all patients in FIG. 3C. These data showed there is a strong linear relationship between RXFP2 expression and the number of apoptotic cells in male patients. There is a moderate positive relationship between RXFP2 expression and the number of apoptotic cells in all 20 patients. There is no relationship between RXFP2 expression and the number of apoptotic cells in female patients.

Expression of cAMP in the ACL Tissues

Relaxin-2 imparts extracellular catabolism in some types by binding to its receptor 1 and 2 as part of a cAMP-dependent pathway. The levels of cAMP were assessed using an enzyme-linked immunoassay for 17 patients. Patient 8, 10, and 17 (2 males and 1 female) were not analyzed due to insufficient cell growth. There was no significant difference found between cAMP concentrations in females and males. The mean cAMP concentration in the 8 male ACLs was 44.22±6.55 pmol/mg, and the mean cAMP concentration in the 9 female patients was 53.86±8.45 pmol/mg (FIG. 3D). There was also no significant correlation found between cAMP concentrations and age. The correlation coefficient r for cAMP and age was 0.2639 (r²=0.06967, p=0.5276) for male patients, r was −0.1789 (r²=0.03199, p=0.6452) for female patients, and r was −0.05598 (r²=0.003134, p=0.831) for patients overall (FIG. 3E and FIG. 3F). These data showed no correlation between cAMP concentration and 20 patients' age. These results indicate that there is no age-dependent change in cAMP concentration in ACL from male and female patients.

Correlations Between Apoptotic Cell Number and Time from Injury

Correlation coefficient r was −0.1357 (r²=0.01841, p=0.7086) for male patients, r was −0.0008151 (r²=6.643e-007, p=0.9982) for female patients, r was −0.08718 (r²=0.0076, p=0.7148) for all patients (FIG. 3G). These data showed there is no relationship between the number of apoptotic cells and time from injury in male patients, female patients and all 20 patients.

### Expressions of MMP1 and MMP13 in Patient ACL Tissues

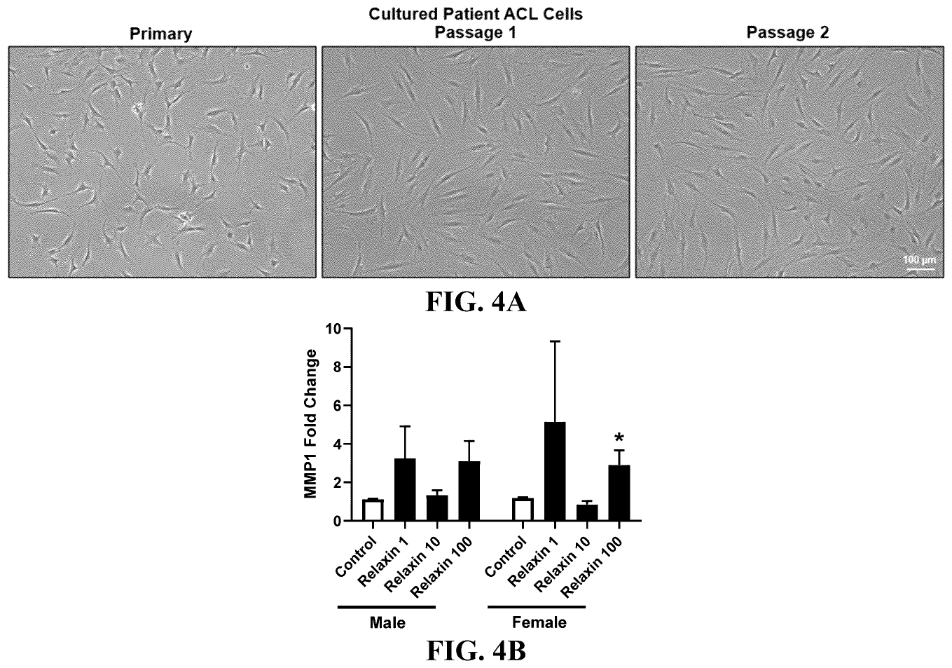

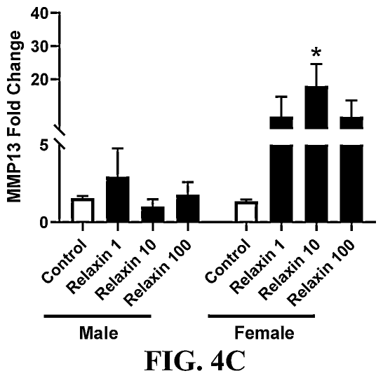

The human ACL cells made progressive morphological changes during culture (FIG. 4A, scale bar at lower right=100 μm). Primary ACL cells were fibroblasts with a dark, round nucleus and short branches. Passaged ACL cells were fibroblasts with clustering, long branches, elongated and web-like shape. In female ACL cells, MMP1 expression increased in response to 100 ng/mL of relaxin-2, while MMP13 expression increased in response to 10 ng/mL of relaxin-2 as compared with untreated controls. In male ACL cells, relaxin-2 treatments didn't significant change the expressions of MMP1 and MMP3 as compared to control groups (FIG. 4B and FIG. 4C).

Results: Both male and female ACL human samples expressed RXFP2 and apoptosis. The number of RXFP2 positive cells was found to be significantly higher in ACL tissues from female patients as compared to that of male patients or normal female tissues. There was a significant correlation between RXFP2 expression and age in male patients, between apoptosis and age, and between RXFP expression and the number of apoptotic cells in 20 patients. Apoptotic expression was found to be significantly higher in female patients compared to that of normal female tissues and male patients. There was no age difference between male and female patients. In female cells, MMP1 expression increased in response to 100 ng/mL of relaxin-2, while MMP13 expression increased in response to 10 ng/mL of relaxin-2 as compared with untreated controls.

The data support that increased expression of RXFP2 and cell apoptosis in the ACL tissues from females may contributes to higher risk for developing ACL rupture after surgical reconstruction. Mechanistically, this is associated increased MMPs expression mediated by relaxin/RXFP2 signal pathway in females.

Clinical Relevance: This study significantly helps a better understanding of sexual difference of RXFP2 expression on patients with ACL reconstruction and provides the evidence to screen and/or develop potential drugs candidates to treat or prevent ACL injury by inhibiting RXFP2.

Relaxin serum concentrations have been found to be significantly higher in Division I female athletes with ACL tears than those without ACL tears, but another study found that serum concentrations of relaxin-2 were comparable in athletic college men and women. There has been very little evidence for the presence of RXFP2 in the male ACL, but consistent evidence of RXFP2 in the female ACL. It was reported that relaxin exhibits specifically saturable binding in human female ACL, where its receptors are observed. Relaxin-2 upregulates intracellular processes in human female ACL cells but has no effect on male cells. Therefore, the high incidence of female ACL rupture may be due to the changes in relaxin-2 levels.

### Discussion

Accordingly, we sought to examine the expression of relaxin-2 receptor in ACL explants from both female and male patients. Further experimental details are discussed in Examples 1-3 below. In this study, we found that there was a significantly higher number of RXFP2 in female patient ACLs as compared to normal females and male patients, suggesting a gender related difference in the activity of relaxin-2 in patients, and RXFP2 expression increased after ACL reconstruction in females. This is the first time to compare the RXFP2 expression in ACLs of patients with ACL reconstruction to that of normal donors. We also found that RXFP2 was present in both male and female patient ACL tissues, providing evidence for the first time that RXFP2 is consistently expressed in male ACLs. In contrast, an earlier study showed that relaxin receptors were not present in male subject ACL.³³

In a prior study, elevated serum relaxin concentrations were detected in female athletes with ACL tears as compared to those females without tears.³² Another study indicated that serum relaxin levels are similar between young athletic men and women.¹⁴⁰ In addition, relaxin exhibited specific saturable binding in the female patient ACLs, where specific relaxin receptors were present.³³ Konopka et al. revealed female athletes with higher circulating relaxin levels may be more susceptible to ACL injury.⁷⁶ Previous studies demonstrated that within the ACL, increased relaxin levels have been found to be correlated with increased risk for ACL injuries, and high numbers of RXFP2 have been observed in female ACLs as compared to no or very minimal RXFP2 number in males. However, proteins of relaxin receptor RXFP1 and RXFP2 were observed in the ACL tissues, achilles tendons, inguinal ligaments, and shoulder joint capsules of male wistar rats.⁷⁴ Relaxin exhibits specific saturable binding in the female patient ACLs, but not in male ACLs.³³

Apoptosis can be triggered by numerous stimuli, such as high temperature, ischemia, immune reactions, hypoxia, exposure to certain drugs and chemicals, infectious agents, radiation, and various disease states.³⁷ Increased levels of apoptotic, inflammatory and catabolic factors in chondrocytes were detected after ACL ruptures.^{24,103} Increased apoptosis has been found to be correlated with increased tissue degradation.⁹¹ Satellite cell apoptosis was significantly increased after ACL reconstruction.¹⁰⁴ ACL cell apoptosis could occur during ACL reconstruction surgical procedure due to higher knee temperature using arthroscopy, or ACL tissue damaged by surgical instruments, and so on. Relaxin-2 was correlated with increased risk of ACL ruptures in females through regulation of key structural components, such as collagen and MMPs.⁷⁶ Following ACL reconstructions, all twenty patients in our study had apoptotic ACL cells. Given that apoptosis and RXFP2 numbers were higher in female patients compared to male patients, it is possible that increased RXFP2 activity enhanced tissue degradation that might be associated with increased apoptosis following the ACL reconstruction due to increased degradation. Our data showed that apoptosis and RXFP2 expression were correlated in male patients and all 20 patients, but not in female patients. A possibility maybe that more RXFP2 expression in female patients could not only induce apoptosis, but also active other signals that cannot be activated in male patients.

Previous research reported that enhanced apoptosis is caused by increased tissue degradation in the ACL, but wasn't able to definitively determine whether apoptosis following mechanical injury in the ACL is the cause or result of degradation.⁹¹ In our study, apoptosis was detected on both male and female patient ACLs. The number of apoptotic ACL cells was significantly positively correlated with patient age overall, which is consistent with the fact that older patients have increased tissue degradation. Considering the number of RXFP2 is weak correlated with female patients' age, but the number of apoptotic cells is significantly correlated with female patients' age, RXFP2 likely is weakly involved in age-related increases in apoptotic activity following ACL injury and reconstruction.

The ACL is a ligament in the center of the knee that helps maintain the knee's rotational stability and prevents the shin bone (tibia) from moving forward on the thigh bone (femur). The ACL is composed of multiple types of collagen bundles and a matrix made of a network of proteins, elastic systems, glycoproteins, and glycosaminoglycans with multiple functional interactions.³⁵ The majority of the ACL is composed of bundles of type I collagen. Type II collagen was observed in the fibrocartilaginous regions of the ACL, specifically the tibial and femoral sites of attachment. Within the ACL, type III collagen is located in the loose connective tissue that divides the type I collagen bundles.³⁵ Among collagenases, MMP1 and MMP13 are the major enzymes that degrade type I collagen.⁵⁹ MMP13 cleaves triple helical collagens, including type I, type II and type III collagen, but shows higher cleavage specificity for type II collagen. MMP13 is five times less potent than MMP1 in cleaving collagen types I and III.¹⁰⁹ Degradation of the main matrix components of ACL could possibly raise the risk if ACL injury.⁷⁶ A significant upregulation of MMP13 was found in patients with ACL rupture.¹⁰³ In addition, recombinant human relaxin-2 markedly upregulated intracellular processes in human female ACL cells, but no effect was detected in male cells. Relaxin-2 significantly increased expression of MMP1 and MMP13 and decreased expression of type I and III collagen in cultured ACL cells, suggesting that relaxin-2 may impact the structural integrity of the ACL, leading to increased risk for ACL ruptures.⁷⁶ Moreover, MMP1 induced apoptosis of myelodysplastic syndromes cells via interaction with protease-activated receptor 1, which further activates p38 MAPK signaling pathway.¹⁴⁶

In osteoarthritis cartilage tissues, silencing MMP13 inhibited cell apoptosis yet increased cell proliferation through upregulation of type II collagen, and downregulation of interleukin-1 (IL-1)β, tumor necrosis factor-α and IL-17. These results suggested that MMP13 could suppress cell proliferation while enhance cell apoptosis.⁸³ However, the effects of relaxin-2 on MMP1 and MMP13 expressions, and apoptosis in patient ACL cells require further investigations as discussed herein. We investigate the potential intracellular mechanisms by which relaxin-2 may be involved in to reduce ACL structural integrity. Some of our findings were consistent with the results reported by Konopka et al. that relaxin-2 administration augmented MMP1 and MMP13 expressions, while no effect was detected on male ACL cells.⁷⁶ There is no significant difference in cAMP levels in female and male ACL cells primed with relaxin-2 and estrogen.⁷⁶ We also found relaxin-2 treatment increased expressions of MMPs in female ACL cells but not male ACL cells, and couldn't change cAMP levels in male and female ACL cells. Further investigations on the mechanistic role of MMP1, MMP13 and apoptosis, and their signaling pathways in human ACLs can be performed. Our results also showed same results with Konopka et al, that there was no significant difference between the cAMP concentration of female ACLs and those of males. In addition, there was no significant correlation between cAMP concentration and age. These findings suggest that cAMP is likely not one of the intracellular mechanisms by which relaxin-2 is involved in regarding the increased degradation and likelihood of ACL injuries for females.

Twenty ACL samples were harvested from patients undergoing ACL reconstructions. A larger sample size would have been more ideal to reduce sampling errors but given the more challenging nature of acquiring human ACL samples, this study limited the sample size to 20 patients. For female patients, we consider that the use of oral contraceptives and the timing of their menstrual cycle may impact the expression of RXFP2 receptors. Recent study has demonstrated that high serum relaxin-2 levels were observed during the luteal phase than in the follicular phase, while oral contraceptives therapy attenuated serum relaxin-2 levels.⁹⁸ Previous investigations showed mixed findings with regard to whether they help protect the ACL against injury.^{77,15,18,39} There is a protective association between oral contraceptives use and the likelihood of sustaining an operatively treated ACL injury.¹¹⁵ Also, oral contraceptives use reduced ACL injury risk.⁷⁷ On the contrary, another study indicated that oral contraceptive administration did not show any protective effect against ACL injuries.¹¹⁵ However, the women had a significantly higher percentage of ACL injuries during midcycle (ovulatory phase) and lower percentage of ACL injuries during the luteal phase of the menstrual cycle. oral contraceptive use diminished the significant association between anterior cruciate ligament tear distribution and the ovulatory phase.¹³⁹ If the female patients are taking oral contraceptives, the RXFP2 levels could appear lower than what is representative of female ACLs without taking oral contraceptives.

Furthermore, some publications have shown that relaxin levels differed during certain periods of the menstrual cycle, but some studies found that relaxin levels increased during the luteal phase following ovulation.^{61,81,139} If the female ACL specimens were harvested during the highest relaxin phase, such as the luteal phase, the female RXFP2 countmight be similarly skewed higher compared to other phases of the menstrual cycle. Further studies should consider a patient's menstrual cycle phase and oral contraceptive use. The results of this study suggest that modulating RXFP2 may offer promising future therapeutic alternatives for females with increased risk of ACL tears. Further long-term studies focused on in vivo RXFP2 therapy and uncovering the precise mechanisms of RXFP2 should be undertaken, to decrease the sex disparity of ACL injury.

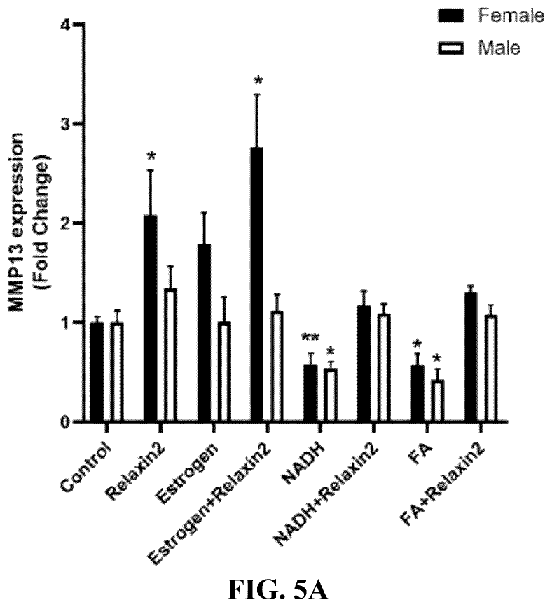

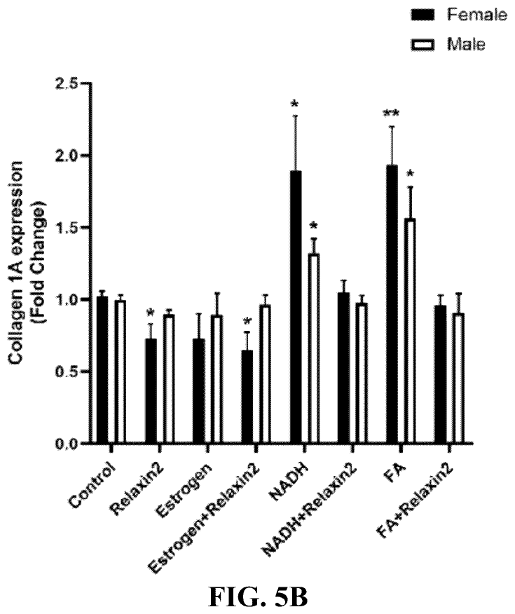

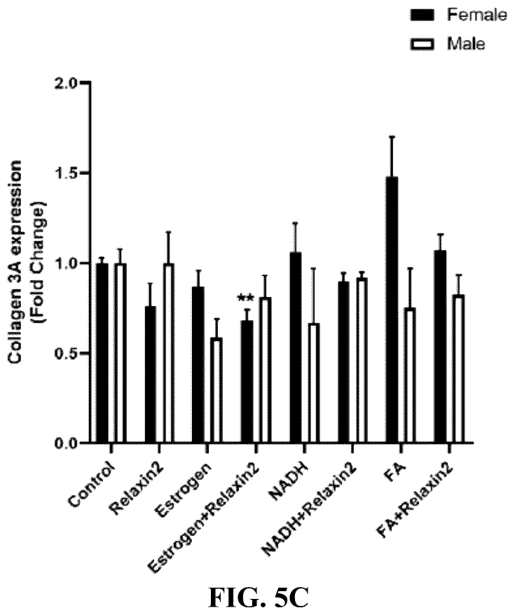

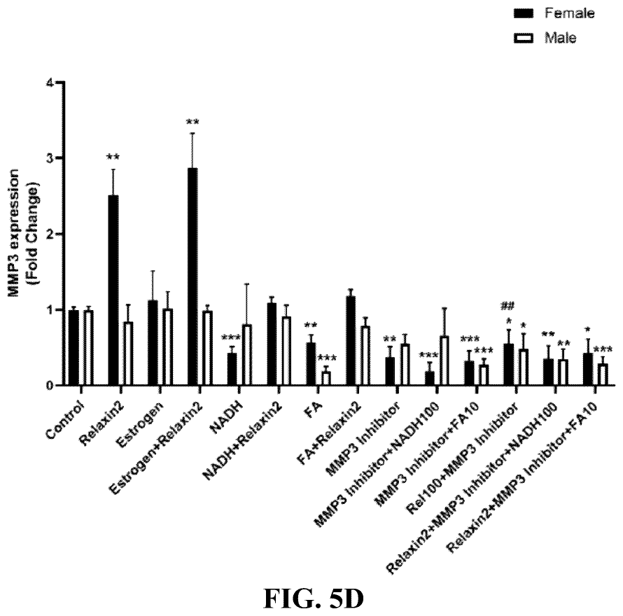

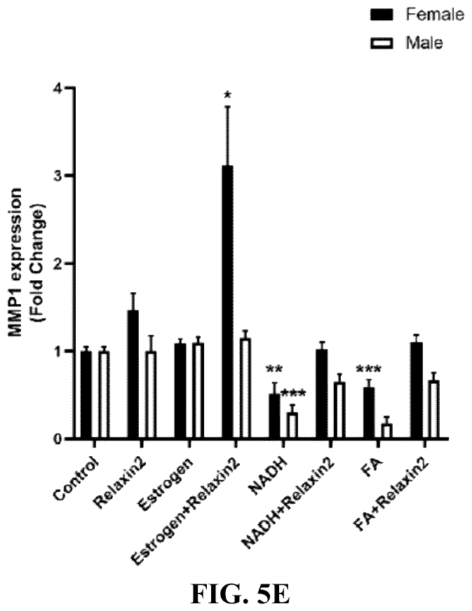

Measurements of expressions of MMP13, collagen 1A, collagen 3A, MMP3, and MMP1 in patient ACL tissues were conducted (FIG. 5). Biopsy tissues of the ACL from 20 patients (10 male and 10 female) who underwent surgeries for ACL reconstruction were harvested. The patients' ages ranged from 16 to 61 years, with equal numbers of males and females. The protocol for patient enrollment was approved by the IRB committee at our institution. Each ACL tissue sample was minced and digested and then the cells were cultured and passaged before treated with different concentrations and combinations of 17β-estradiol (1 μM), relaxin-2 (100 ng/mL), relaxin-2 antagonist (Folic Acid, 10 μg/ml), and relaxin-2 receptor antagonist (NADH, 100 μg/ml). After treatment, cells were lysed and analyzed for the expressions of type I collagen, type III collagen, MMP1, MMP3, and MMP13 using quantitative real-time polymerase chain reaction (RT-PCR). Examples of expressions of MMP13, collagen 1A, collagen 3A, MMP3, and MMP1 in patient ACL tissues are presented in FIG. 5. MMP13 expression (FIG. 5A); collagen 1A expression (FIG. 5B); collagen 3A expression (FIG. 5C); MMP3 expression (FIG. 5D); and MMP1 expression (FIG. 5E), all present the innovation. The protocol for patient enrollment was approved by the IRB committee at Rhode Island Hospital. GraphPad PRISM 8 software was used to perform all statistical analyses. Results were presented as mean+standard error of the mean (SEM) of different samples and considered significant when p<0.05. A t-test was used to compare any two groups, *p<0.05, **p<0.01, ***p<0.001 compared to their individual control group; ##p<0.01, compared to their individual relaxin-2 group.

As discussed further in the Examples 1-3 below, we examined the utility of Relaxin-2 and its receptor blockers to improve the injury prevention characteristics of the extracellular matrix. Ongoing work is further described in the Examples, as well as application to other readily discernable areas (e.g., shoulder work). For examples, we chose to test 2 commonly used molecules—Folic Acid (FA) and NADH, whose safety is established. We exposed fibrocytes from explanted human anterior cruciate ligament (ACL) specimens and found that the administration of these antagonists for relaxin-2 resulted in expression of proteins associated with an improved extracellular matrix milieu—a significant decrease in MMP-13 and significant increases in collagen 1A and 3A expression.

Expression of a Relaxin-2 Receptor Rxfp2 in the Shoulder Capsule and Labrum of Patients with Shoulder Instability

Background: Elevated serum relaxin levels have been shown to correlate with the risk of shoulder instability. Although a relaxin receptor RXFP2 is expressed in shoulder joint capsule of rats, whether RXFP2 is present in the capsule and labrum of patients with shoulder instability remains unknown.

Methods: Biopsy tissues of the shoulder labrum and capsule were obtained from 20 patients (ages between 18 and 40 years old, 15 male and 5 female) undergoing surgical treatment of shoulder instability. These tissues were fixed in 4% paraformaldehyde and embedded in paraffin blocks, then 5 m sections were cut. Immunohistochemistry was performed to detect the expression of RXFP2, and number of RXFP2 positive cells from 5 to 7 different fields was counted. Experimental details are further discussed in Example 5 below.

Results: RXFP2 was expressed in capsules and labra of patients with shoulder instability. The number of RXFP2 positive cells was higher in male tissues than those in female tissues. We also found that RXFP2 was present in vessels of capsules and labra in patients with shoulder instability. There was no correlation between RXFP2 expression and patients' age.

These data support that RXFP2 was expressed in capsules and labra of patients with shoulder instability, which was higher in male tissues than those in female tissues.

### Introduction

Shoulder instability is common among people who participate in contact and noncontact athletic activities. Shoulder instability usually occurs when the capsule or labrum become stretched, torn or detached from the bone. Surgical intervention is often used to repair the capsule, labrum and other soft tissues surrounding the joint and to limit future instability episodes.^{36,72} While the shoulder capsule and labrum have been the focus of many surgical studies in humans, the molecular factors that affect post-injury changes are not fully understood.

Relaxin is a peptide hormone that is a member of the insulin-like superfamily. Seven relaxin family peptides (relaxin-1, 2, 3, and the insulin-like peptide (INSL) 3, INSL 4, INSL 5, and INSL 6) are all structurally related to insulin, and they produce their physiological effects by activating a group of G protein coupled receptors: relaxin family peptide receptors (RXFP), including RFXP1, RFXP2, RFXP3, and RFXP4.¹⁰ Relaxin plays critical roles in biological processes, such as growth, metabolism, pregnancy, and parturition.^{10,11} Among these relaxins, relaxin-2 is best studied and is the only known relaxin that can circulate in the blood.^{124,137} Interestingly, serum relaxin levels are similar between young athletic men and women.¹⁴⁰ Furthermore, relaxin receptors are present at the ligament, cartilage, and synovium of the trapeziometacarpal joints from both male and female patients.²² Relaxin-2 activates two orphan G protein coupled receptors: RXFP1 and RXFP2.⁵⁶ RXFP1, also known as leucine-rich repeat-containing G-protein coupled receptor (LGR)7, and RXFP2 (also known as LGR8) are present in ligament, tendon, and shoulder joint capsule of male rats.⁷⁴ Although a previous study showed that an elevated serum relaxin level correlates with the risk of shoulder instability,¹⁰⁰ the expression of the relaxin-2 receptor in the human capsule and labrum has not been reported. Based on the reports mentioned above, we investigate the extent relaxin-2 receptors are differentially expressed in shoulder capsules and labra between male and female with shoulder instability. Example 5 below presents experimental details of this study.

RXFP2 was Expressed in Shoulder Capsules and Labra from Both Male and Female Subjects.

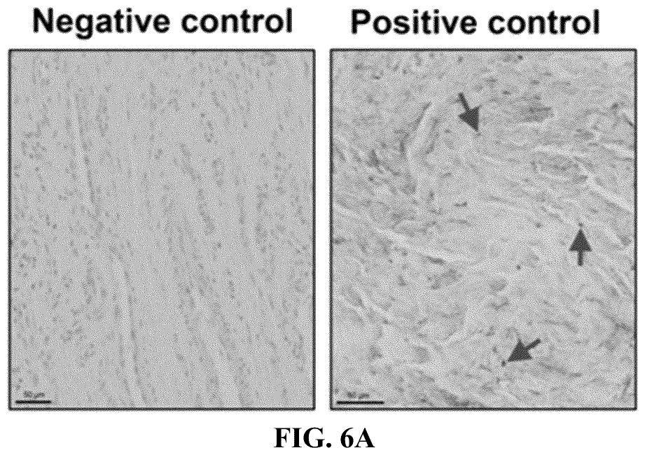

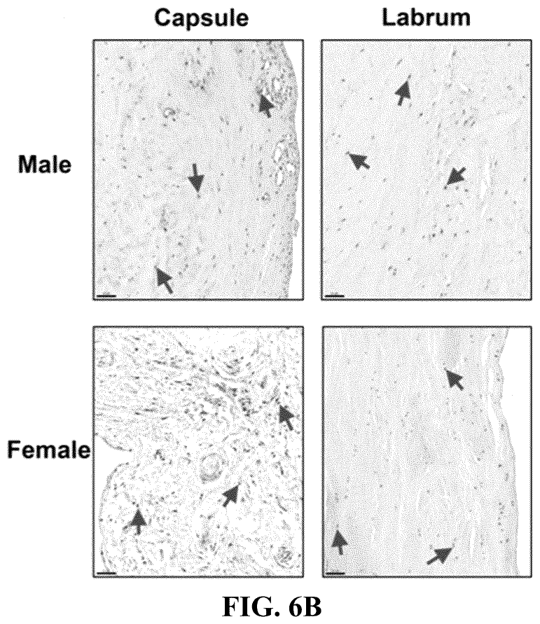

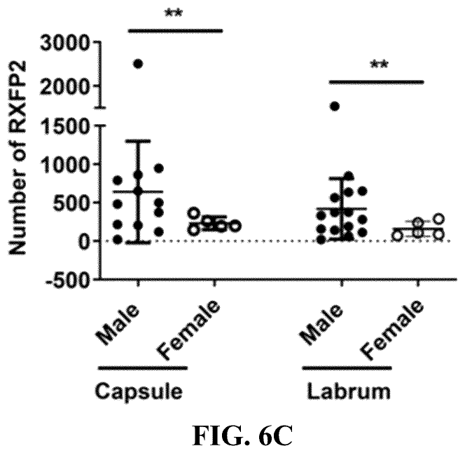

FIG. 6 shows data for expression of RXFP2 on shoulder capsules and labra from 20 patients with shoulder instability. Immunohistochemical staining of a relaxin-2 receptor RXFP2 were performed in shoulder capsule and labrum from all 20 male and female patients with shoulder instability. The brown precipitate (gray in grayscale figures) indicates the presence of the target antigen RXFP2. The nucleus is stained purple by hematoxylin staining. Combination of purple and brown colors resulted in dark brown (darker gray). The brown and dark brown cells were counted as RXFP2 positive cells. FIG. 6A (left panel) showed a negative control that was not incubated with a primary RXFP2 antibody during staining in human labrum. It has been reported that RXFP2 were present in shoulder joint capsule of young male Wistar rats.⁷⁴ Thus, we used shoulder labrum of male Wistar rat to stain RXFP2 as a positive control (right panel in FIG. 6A). Negative control in human labrum tissues was not incubated with a RXFP2 antibody. RXFP2 immunohistochemistry in shoulder labrum of male Wistar rat was used as a positive control. (FIG. 6B) Representative immunohistochemistry images of RXFP2 positive cells in capsule and labrum tissues of patients with shoulder instability.Grayarrows indicate RXFP2 positive cells (brown to dark brown precipitate). (FIG. 6C) Quantification of the number of RXFP2 positive cells in 1 mm² field from male patients (n=15) and female patients (n=5). Results were presented as mean±SD of different samples, as the dots are absolute number of RXFP2 positive cells. **P<0.01 vs male. As shown in FIG. 6B, RXFP2 was expressed in both shoulder capsules and labra from patients with shoulder instability. The number of RXFP2 positive cells in 1 mm² field was 638±191 in male capsule, 230±37 in female capsule, 417±102 in male labrum, and 179±42 in female labrum. Statistical analysis showed that a significantly higher number of RXFP2 positive cells in 1 mm² field was observed in capsules and labra in male than those in female from patients with shoulder instability (FIG. 6C). These results suggest that RXFP2 is expressed in both shoulder capsules and labra, and this is higher in male patients with shoulder instability compared to female subjects.

RXFP2 was Expressed in Blood Vessels and Nucleus of Shoulder Specimens from Male and Female Patients.

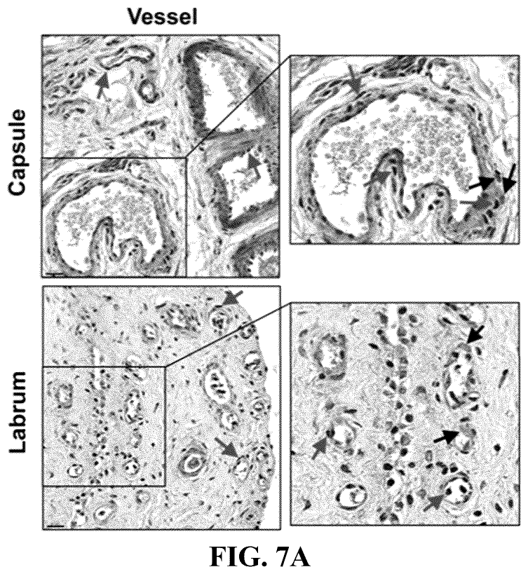

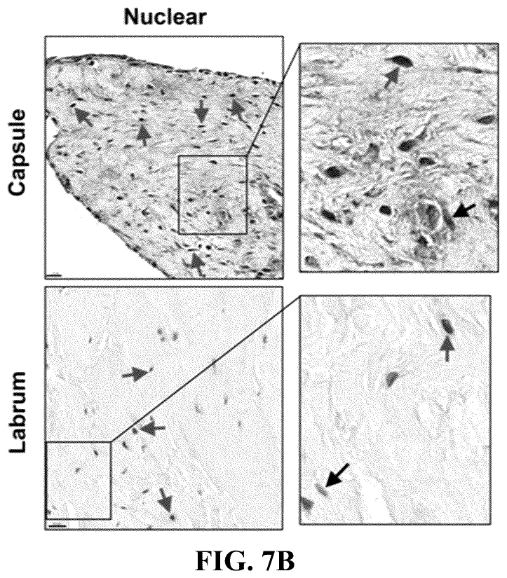

In FIG. 7A (upper panel), RXFP2 was widely expressed in blood vessels from the capsules of male and female patients with shoulder instability. In addition, RXFP2 was present in nucleus of capsules and labra of patients with shoulder instability (FIG. 7B). Nuclear expression of RXFP2 was shown as a dark brown staining after combination of purple (nuclear) and brown (RXFP2 positive) colors (darker shaded gray in grayscale). These results demonstrate that RXFP2 can be expressed in vessels and nucleus of patients with shoulder instability. FIG. 7 demonstrates expression of RXFP2 in vessels and nucleus in shoulder specimens from patients with shoulder instability. Immunohistochemical staining of a relaxin-2 receptor RXFP2 were performed in patient shoulder specimens. The brown precipitate (gray) indicates the presence of the target antigen RXFP2. The nucleus is stained purple (darker gray in grayscale) by hematoxylin staining. Combination of purple and brown colors resulted in dark brown. The brown and dark brown cells were counted as RXFP2 positive cells. Representative immunohistochemistry images of RXFP2 located in vessels (FIG. 7A) and nucleus (FIG. 7B) of capsules and labra in patients with shoulder instability. Gray arrows (formerly red in color images) indicate RXFP2 positive immunostaining (brown to dark brown precipitate). Black arrows indicate RXFP2 negative immunostaining (purple color).

RXFP2 Expression Did not Correlate with Patients' Age

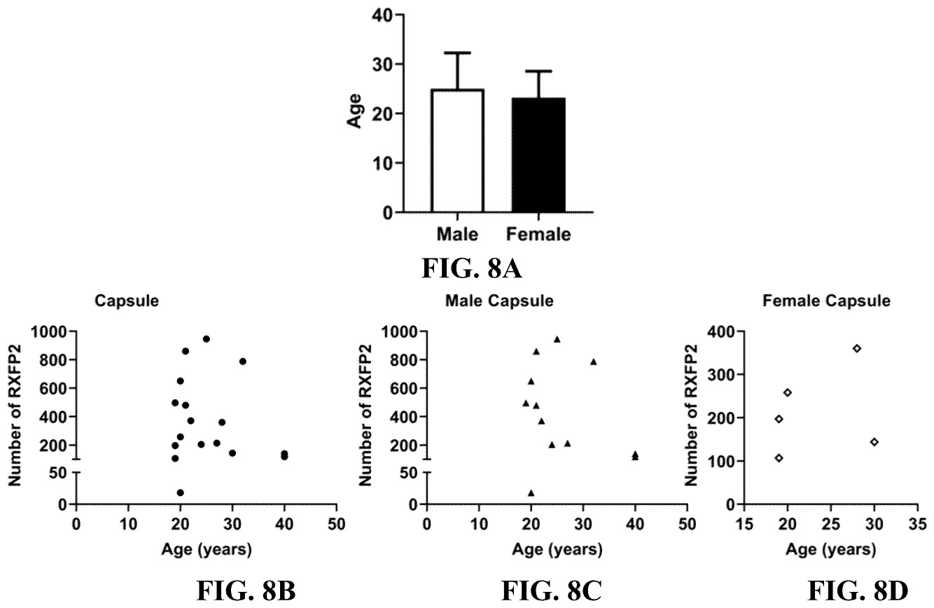

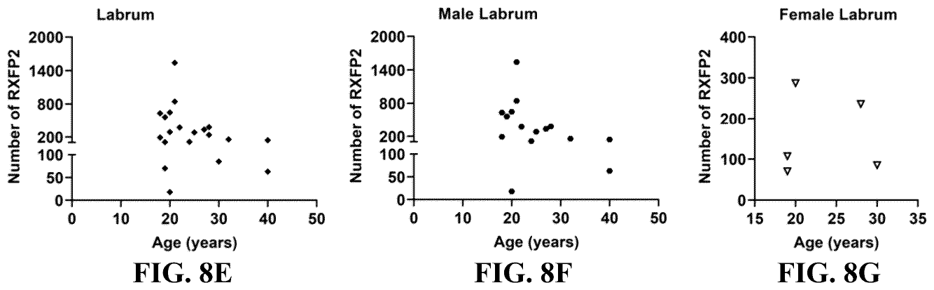

The mean age of 15 male patients was 25.0±1.9, and mean age of 5 female patients was 23.2±2.4. There was no statistic difference in age between male and female patients with shoulder instability (FIG. 8A). The number of RXFP2-positive cells varied on capsules (FIGS. 8B-D) and labra (FIGS. 8E-G) within these 20 patients. We then determined whether there is a correlation between RXFP2 expression and ages in patients with shoulder instability. As shown in FIG. 8B, correlation coefficient (r) between number of RXFP2 positive cells in capsules and ages was −0.1658 (p=0.5247). The r values between number of RXFP2 positive cells in capsules and ages in male and female patients were −0.3090 (p=0.3285) and 0.2826 (p=0.6450), respectively (FIG. 8C and FIG. 8D). The r value between number of RXFP2 positive cells in labra and ages was −0.3364 (p=0.1470) (FIG. 8E). The r values between number of RXFP2 positive cells in labra and ages in male and female patients were −0.4331 (p=0.1068) and −0.02334 (p=0.9703), respectively (FIG. 8F and FIG. 8G). These results indicate that there was no statistical significance in correlation between RXFP2 expression in capsules and labra and patients' age, regardless of sex. FIG. 8 provides data there was no correlation between RXFP2 expression and ages in 20 patients with shoulder instability. Immunohistochemical staining of a relaxin-2 receptor RXFP2 were performed in shoulder capsules and labra from 20 patients with shoulder instability. The number of RXFP2 positive cells in 1 mm² field were counted and the correlation between age and RXFP2 number in the male and female patients were calculated. (FIG. 8A) The age of male (n=15) and female (n=5) patients with shoulder instability was present. The correlation between age and RXFP2 number in 1 mm² field on capsules (FIG. 8B) of patients including twelve male (FIG. 8C) and five female (FIG. 8D) patients. The correlation between age and RXFP2 number in 1 mm² field on labra (FIG. 8E) of patients, including fifteen male (FIG. 8F) and five female (FIG. 8G) patients. The dots (symbols on plots) are absolute number of RXFP2 positive cells for each group.

### Discussion

Relaxin is a protein hormone first described in 1926 by Frederick Hisaw.⁹³ Relaxin plays important roles in the central nervous system,¹⁹ cardiovascular system,¹²² reproductive system,⁶⁹ and musculoskeletal system²⁸ by binding to its receptors in different types of tissues. In females, relaxin emanates from the corpus luteum²⁵ and the placenta⁴⁷ during pregnancy and the ovaries during the luteal phase of the menstrual cycle.⁶⁸ In males, relaxin is mainly expressed in the prostate³, which can be secreted into the seminal fluid, and its receptors are detectable in several male reproductive organs.⁶⁹ Specific to the musculoskeletal system, relaxin had effects on bone, cartilage, synovial lining, muscles, tendons, and ligaments.²⁸ Relaxin correlates with a decrease in knee articular cartilage stiffness and degradation of fibrocartilage via an increase in collagenase and matrix metalloproteinases (MMPs).^{17,60} Moreover, relaxin is an osteoclast-activating factor involved in increasing bone resorption,³⁸ and it is correlated with a decrease in knee articular cartilage stiffness and degradation of fibrocartilage via an increase in collagenase and MMPs.^{17,60} Diseases affecting the synovium, such as rheumatoid arthritis, have shown a lower incidence and severity during pregnancy when relaxin concentrations are high.^{26,63} This could be due to relaxin's anti-inflammatory capabilities in downregulation of neutrophil function⁶ as well as reduced circulating TNF-α and increased IL-10 when combined with estrogen.^{43,21} Furthermore, relaxin was implicated in muscle fiber regeneration and suppressing fibrosis, both of which require an imbalance towards relaxin between relaxin and TGF-3.^{46,55,87,89,35} With respect to tendons, relaxin may play a role in controlling the length of tendons^{15,42} and reducing tendon stiffness via an increase in laxity through activation of collagenase.¹⁰⁶ Lastly, relaxin is implicated in altering ligament mechanics via collagenolytic effects, and also may engage in reducing the density and organization of collagen bundles¹²⁹ due to its activation of collagenase,^{50,38} MMPs,¹¹³ and plasminogen activator.⁷⁵ In the female rats and pigs, relaxin has effects on elongation of the interpubic ligament during pregnancy.¹²⁵

Additionally, relaxin modulates mechanical properties of the anterior cruciate ligament (ACL) in adult female guinea pigs.³⁴ Moreover, relaxin exhibits specifically saturable binding in human female ACL, where its receptors are observed.³³ Shoulder instability is a condition in which the laxity of the shoulder increases abnormally and is frequently characterized by a loose shoulder joint. A previous study demonstrated that serum relaxin level was increased in patients with shoulder instability.¹⁰⁰ Therefore, relaxin could affect post-injury changes in patients with shoulder instability through regulating downstream signaling pathways. However, whether relaxin-2 and its receptors play an important role in patients with shoulder instability is still unknown. In present study, we employed the shoulder capsules and labra from male and female patients with shoulder instability, and determined the expression of a relaxin-2 receptor RXFP2. Here, we found that a relaxin-2 receptor RXFP2 was present on both labral and capsular biopsies taken from male and female patients with shoulder instability. Therefore, it is possible that relaxin-2 and its receptors play important roles in modulating shoulder stability.

Relaxin-2 is expressed in the placenta, decidua, prostate, corpus luteum, endometrium, mammary glands, heart, brain,¹⁰⁰ and ovary during pregnancy.⁴⁸ In the phase III clinical trials for the treatment of acute heart failure, serelaxin, a recombinant form of human relaxin-2, was used as a 48 hour intravenous infusion at a dose of 30 μg/kg/day and showed improvement in short-term dyspnea scores and 180-day mortality compared with placebo group.¹³² To date, research has implicated minimal male RXFP presence in ACL thus limiting the relaxin potential therapeutic population to females.^{33,39,76} Among females, ACL injury rates are higher during the luteal phase of menstruation when relaxin is higher.⁹² Although we found the number of RXFP2 positive cells was higher in male tissues than those in female tissues, we should consider the relationships between the menstrual cycle and RXFP2 expression in our female patients. One study has demonstrated that serum relaxin-2 concentrations were significantly higher during the luteal phase than in the follicular phase, while oral contraceptive therapy reduced serum relaxin-2 in elite female athletes.⁹⁸ Another studies have shown that serum levels of relaxin were detected during both the follicular and luteal phase of the menstrual cycle,^{107,143} while the mean relaxin levels were higher during oral contraceptive use than during the non-oral contraceptive cycle.¹⁴3 Therefore, the menstrual cycle phase and oral contraceptive therapy may affect the expression of relaxin-2 receptor RXFP2 in our female patients.

Relaxin-2 is the only relaxin known to circulate in the blood,^{124,137} and that RXFP2 is present in shoulder joint capsule of young male Wistar rats.⁷⁴ The shoulder joint is supplied by the anterior and posterior circumflex humeral arteries, which are both branches of the axillary artery. Thus, axillary artery is the major blood vessel in the shoulder. Therefore, we determined whether RXFP2 is expression in vessels in capsules and labra of patients with shoulder instability. RXFP1 has been identified in both males and females in the dorsoradial ligament, synovium, and articular cartilage of the trapeziometacarpal joint.²² Relaxin receptor transcripts were also identified in males and females in the anterior oblique ligament of the thumb carpometacarpal joint.¹⁴¹ With respect to shoulder tissues, RXFP1 and RXFP2 expression have been found in shoulder joint capsules in male Wistar rats.⁷⁴ Although there has been limited research in showing the presence of RXFP1 receptor in the shoulder joint and periphery of female rat, intraarticular injection of relaxin-2 alleviated shoulder arthrofibrosis in female rats.¹⁵ Therefore, we decided to investigate if RXFP2 is expressed in human shoulder capsule and labrum from both males and female patients with shoulder instability. We found RXFP2 was present in shoulder capsules and labra from both male and female patients with shoulder instability. Our findings were consistent with Kim's findings that RXFP2 was present in male capsules,⁷⁴ even though Kim et al. found RXFP2 in male rats, and we detected RXFP2 in human males. Dragoo et al. demonstrated that relaxin receptors were only present in ACL tissue from female patients who underwent ACL reconstruction, but not in male ACL tissue.³³ The possible explanations for the different finding in male specimens between ours and Dragoo et al. are as follows: the method of detection of relaxin receptors, and the different human tissues used in the study. Dragoo et al. used biotinylated relaxin and a 2000-fold excess of insulin to do immunohistochemistry to investigate the expression of relaxin receptors in human ACL tissues, but we and Kim et al. use commercial RXFP2 antibody to carry out immunohistochemical staining in human shoulder tissues or rat shoulder tissues. The mechanisms underlying increased RXFP2 expression in capsules and labra in male compared to those in female patients remain elusive.

RXFP2 mRNA expression has been identified in human in the brain, pituitary, uterus, testis, kidney, thyroid, muscle, peripheral blood cells and bone marrow, as well as in rat and/or mouse in the ovary and gubernaculum.⁵⁶ We were interested in finding the location of RXFP2 in shoulder samples. Our findings are in agreement with a previous study showing RXFP2 expression in rat vessels.⁷⁴ Cell-specific expression of RXFP2 in capsules and labra in patients with shoulder instability is investigated herein. Hematoxylin was used to stain cell nuclei purple. In DAB-based staining, brown precipitates indicated positive staining of RXFP2. Combination of purple and brown colors resulted in dark brown. It was found that many dark brown precipitates appeared in shoulder tissues, suggesting that RXFP2 was expressed in the nucleus in capsules and labra from both male and female patients. Nevertheless, further study using immunofluorescence with DAPI staining will confirm it expression in nucleus. During the organ development, the expression of RXFP2 gene increased with age in gubernacular, kidney, testis, and lung.⁴⁰ There was no correlation between relaxin-2 receptor expression and patients' age in the present study. This suggests that age does not affect RXFP2 expression in capsules and labra of patients with instability.

Future planned work for this study is to include a control group in patients without shoulder instability. Further study using shoulder capsules and labra from control subjects (e.g., rotator cuff tear patients, or shoulder arthritis patients) will determine whether RXFP2 was altered during the progress of shoulder instability. In addition, further study with more patients will confirm our findings. Relaxin-2 is a native peptide with high specificity, excellent safety profile, and no major adverse effects reported. The signaling of RXFP2 is regulated by G proteins leading to stimulation of adenylate cyclase and an increase of cyclic adenosine monophosphate.⁴¹ Modulating RXFP2 may offer promising future therapeutic alternatives for subjects with increased risk of shoulder instability. Further long-term studies on in vivo RXFP2 therapy and uncovering precise mechanisms underlying the effects should be undertaken.

Before discussing the inter-combined effects of vitamin D, in conclusion, RXFP2 was expressed in capsules and labra of patients with shoulder instability. This was further augmented in male subjects compared to those in female patients. There was no correlation between RXFP2 expression and ages in these patients. This study helps us better understand the effect of RXFP2 on shoulder instability in human and provides the evidence to develop potential drugs candidates to treat or prevent shoulder instability by targeting RXFP2.

A Diagnosis of Vitamin D Deficiency is Associated with Increased Rates of Anterior Cruciate Ligament Tears and Reconstruction Failure

Further studies were conducted to inter-combine vitamin D effects with the technology disclosed herein. An exemplary purpose of this study was the aim to characterize the association between a diagnosis of hypovitaminosis D and primary anterior cruciate ligament (ACL) tear, primary ACL reconstruction (ACLR), and revision ACLR in different sex and age cohorts.

General overview of methods: In this retrospective cohort study of the PearlDiver claims database, records were queried between Jan. 1, 2011 to Oct. 31, 2018 for all patients aged 10 to 59 who received a diagnosis of hypovitaminosis D. Rates of primary ACL tears, primary reconstruction, and revision reconstruction were calculated for sex- and age-specific cohorts and compared to a control of patients without a diagnosis of hypovitaminosis D. Incidence rates for primary ACL injuries were calculated, and multivariable logistic regression was used to compare rates of ACL injury, primary reconstruction, and revision reconstruction while controlling for age, sex, Charlson comorbidity index, and several other comorbidities.

Overview of Results: Among the 328,011 patients (mean age 41.9±12.6 years, 65.8% female) included in both the hypovitaminosis D and control cohorts, the incidence of ACL tears was 115.2 per 100,000 person-years (95% CI, 107.2-123.7), compared to 61.0 (95% CI, 55.2-67.2) in the demographics and comorbidity matched control cohort. The study cohort was significantly more likely to suffer an ACL tear over a one- (aOR=1.67, 95% CI, 1.41-1.99, p<0.001) and two-year (aOR=1.81, 95% CI, 1.59-2.06, p<0.001) period. This trend remained for both males at the one- (aOR=1.66, 95% CI, 1.29-2.14, p<0.001) and two-year (aOR=1.68, 95% CI, 1.37-2.06, p<0.001) mark and females at the one- (aOR=1.69, 95% CI, 1.33-2.14, p<0.001) and two-year (aOR=1.80, 95% CI, 1.51-2.14, p<0.001) mark. Finally, vitamin D-deficient patients had a significantly increased likelihood of undergoing a revision ACL reconstruction within two years of a primary reconstruction (aOR=1.28, 95% CI, 1.05-1.55, p=0.012).

Overview of conclusions: This study reports an association between patients previously diagnosed with hypovitaminosis D and significantly increased rates of both index ACL tears (81% increase within two years of diagnosis) and revision ACLR (28% within two years). These results identify a population with increased odds of injury and provide valuable knowledge as we expand our understanding of the relationship between vitamin D and musculoskeletal health.

### Introduction

Anterior cruciate ligament (ACL) injury is one of the most common knee injuries, accounting for a large physical, psychological, and economic burden on affected patients. Injury incidence and subsequent surgical repair are increasing, with tear rates approaching 250,000 per year and up to 175,000 of those undergoing ACL reconstruction (ACLR).^{21,53,84,119,130} Several risk factors for ACL rupture have been studied, including female sex, high-risk athletic activities, young age, genetic predisposition, body mass index (BMI), and hormonal fluctuations.² Previously, decreased vitamin D levels have been implicated in poor musculotendinous health.^{9,20,57,71} Both Harada et al. and Cancienne et al. have found that reduced vitamin D levels are associated with poor operative outcomes following rotator cuff repair.^{20,57} Similarly, Barker et al. reported impaired strength recovery in vitamin D-deficient patients undergoing ACL reconstruction.⁹ In a separate study, Barker et al. also reported an association between higher vitamin D concentration and faster strength recovery following a muscular injury.⁷ Other studies have demonstrated that vitamin D supplementation in deficient patients resulted in increased muscle strength and athletic performance, as well as decreased circulating levels of matrix metalloproteinase-9 (MMP-9), a known contributor to connective tissue degradation and pathogenic remodeling.^{4,131,133} However, it should be noted that there is no high-quality evidence suggesting that alterations in MMP-9 concentrations directly affect ACL injury risk.

In ACL-deficient patients who desire to return to sports or occupational activities that require cutting and pivoting, ACLR remains the gold standard for surgical management.¹³⁰ Despite ongoing advancement regarding surgical technique and graft choice, a history of ACL injury remains a notorious risk factor for a second ACL injury (including ipsilateral and contralateral), with reports of graft failure near 9% and secondary ipsilateral revision or contralateral reconstruction nearing 40% longterm.^{51,20} Although conflicting data exist, a recent meta-analysis by Zhao et al. reported several prognostic factors for failure of ACLR, including male sex, younger age, lower BMI, concomitant MCL injury, hamstring autograft use, allograft use, tunnel malposition, and smaller graft diameter.¹⁴⁵

Although many factors for ACL injury and ACLR failure are non-modifiable, further investigation into common and potentially modifiable risk factors is needed. Hypovitaminosis D is a major public health issue, with nearly 1 billion people afflicted worldwide.^{64,95} Though widely prevalent, vitamin D levels are easily modifiable and often treated simply with daily supplementation after an initial loading dose to restore and maintain normal physiologic levels of vitamin D.⁷³ Numerous studies have demonstrated the association between physiologic vitamin D levels and improved regulation of the inflammatory cascade as well as promotion of neuromuscular function in orthopedic patients.^{16,86,88,101} The existing literature on ACL injuries and vitamin D deficiency, however, is sparse, contradictory, and comprised of small cohort studies and animal research.^{54,88} This study aims to characterize the association between a diagnosis of hypovitaminosis D and primary anterior cruciate ligament (ACL) tear, primary ACL reconstruction (ACLR), and revision ACLR in different sex and age cohorts. It was hypothesized that an association between a diagnosis of vitamin D deficiency and higher rates of primary ACL injury, primary ACLR, and revision ACLR would be identified.

### Methods

Data Source—This retrospective comparative analysis was performed using de-identified data from both the M151Ortho (all patients included in PearlDiver) and MArthro (all patients who underwent an arthroscopic procedure within PearlDiver) datasets within the PearlDiver (PearlDiver Technologies, Colorado Springs, CO) database from Jan. 1, 2011 to Oct. 31, 2018. This insurance claims database is generated and updated using all Humana Incorporated insurance claims from over 151 million insured patients from January 2010 through October 2020. These data provide researchers the ability to longitudinally characterize and analyze short-, medium-, and long-term rates of various conditions and complications using International Classifications of Disease, ninth (ICD-9) and tenth revision (ICD-10) and Current Procedural Terminology (CPT) codes. For the current study, these data were used to calculate and compare rates of index ACL tears, primary ACLR, and revision ACLR in those who underwent surgery with vitamin D deficiency and a matched control population.

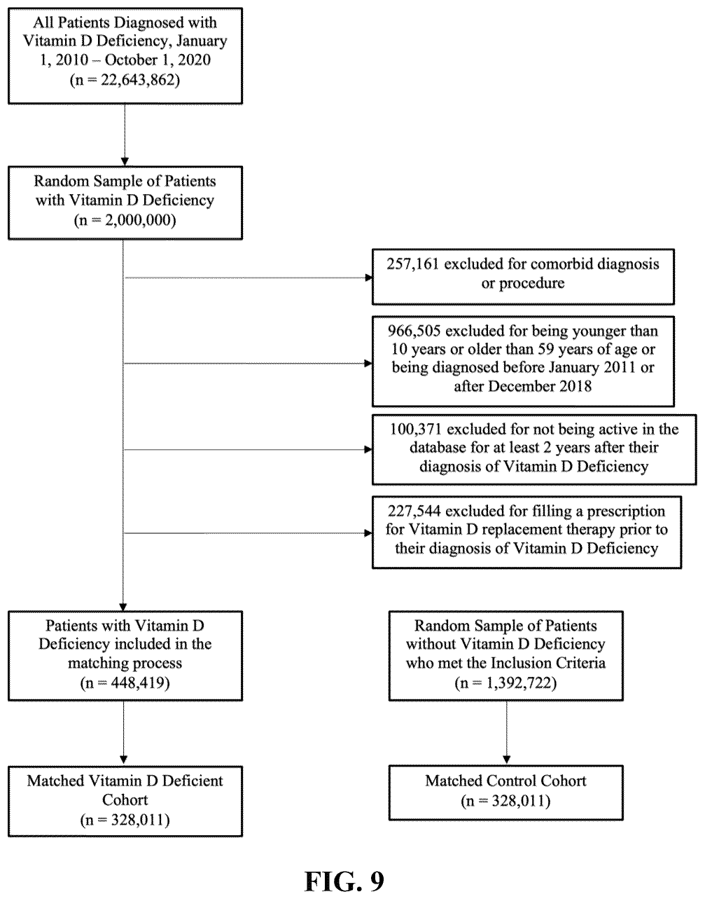

Generating the Cohorts: The M151Ortho dataset within the larger Mariner database was queried for all patients with a diagnosis of Vitamin D deficiency (ICD-9-2689 and ICD-10-E559). This query returned 22,643,862 patients (˜15%), and to facilitate data handling, a randomly generated sample of 2,000,000 of these patients was used to create the initial vitamin D-deficient cohort. Subsequently, this sample was filtered to isolate those patients aged 10 to 59 with a diagnosis of vitamin D deficiency between Jan. 1, 2011 to Oct. 31, 2018. This age range was selected as it contains most patients with index ACL injuries, and this time period was selected to allow for a minimum of data one year before and two years after the diagnosis of vitamin D deficiency.¹¹⁹ Those patients who were not active within the dataset, meaning they changed providers or insurance and are no longer tracked within the dataset, for this three-year span were excluded from the study. Patients with a diagnosis of connective tissue or rheumatologic disease, mitochondrial disease, Paget disease of the bone, multiple myeloma, hyperparathyroidism, cachexia, a malabsorptive disease like inflammatory bowel disease, achalasia, or those who underwent a previous bariatric surgery were excluded from the study. Patients who filled prescriptions for vitamin D supplementation were also excluded. This cohort was subsequently matched using the 1:1 exact matching method based on age, sex, Charlson comorbidity index, tobacco use, diabetes, osteoporosis, and overweight/obesity (body mass index (BMI)>25) to a randomly generated control cohort that met the inclusion criteria. This created two overall cohorts of 328,011 patients that were subdivided into sex-specific and age-specific (10 to 25, 26 to 40, and 41 to 59 years) sub-cohorts (FIG. 9).

Calculating the Rate of ACL Tears and Primary Reconstruction: The rate of ACL tears was calculated over a one- and two-year period following the diagnosis of vitamin D deficiency and compared to the same time periods within the control group. These injuries were identified using ICD-9 (71783) and ICD-10 (M23611, M23612, M23619, S83511A, S83511D, S83511S, S83512A, S83512D, S83512S, S83519A, S83519D, S83519S) codes. Only index ACL tears were included in the rate analysis. The rates of primary ACLR in both cohorts were identified using CPT-29888 and calculated using the number of index ACL tears over the two-year period previously determined.

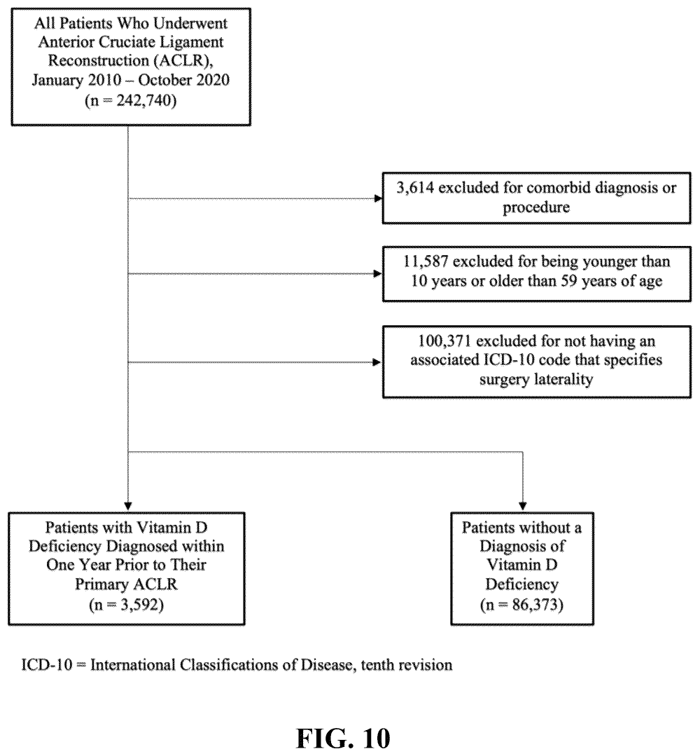

Calculating the Rate of Revision ACL Reconstruction: In this second analysis of revision ACLR, the MArthro sub-dataset within the larger Mariner database was queried for all patients who underwent arthroscopic ACL reconstruction (CPT-29888), and the previously utilized exclusion and inclusion criteria were applied, creating a new sample population. Only index ACLR was included in the analysis. Only patients who underwent ACLR and had an associated ICD-10 code that specified laterality (M23611, S83511A, S83511D, 5835115, M23612, 583512A, 583512D, 5835125) were included in this part of the study. This allowed for the identification of which knee was operated on and limited the analysis to only those patients who underwent a revision ACLR on the same knee previously operated on, excluding those patients who subsequently underwent ACLR on the opposite knee. This cohort was divided based on the presence of a diagnosis of vitamin D deficiency within the year prior to the primary ACLR (FIG. 10). To ensure proper power in the subsequent analysis, 1:1 exact matching was not performed, but all variables included in the previously described matching process were included in the multivariable regression analysis to control for potential confounding, replacing the role of matching in this analysis. Rates of revision ACLR within one and two years of the initial surgery were calculated and compared.

Statistical Analyses: The incidence of ACL tears was calculated for all cohorts in units per 100,000 person-years by dividing the number of ACL tears that occurred over the two-year period by the number of patients at risk over that two-year period. These rates were compared using the exact Poisson method. Multivariable logistic regression, controlling for age, sex, CCI, diabetes, tobacco use, chronic kidney disease, overweight or obesity (BMI>25), morbid obesity (BMI>40), and osteoporosis, was used to compare rates of ACL tears, primary reconstructions, and revision reconstructions. Adjusted odds ratios (aOR) and 95% confidence intervals (CI) were generated and reported for each comparison. To protect patient identity, cohorts comprised of less than 11 patients are reported as “−1” by PearlDiver and thus were reported as “<11” throughout this manuscript. Statistical analyses are still able to be performed on these smaller cohorts, but the specific value is simply not reported. A p-value of <0.05 was determined to represent statistical significance a priori. All statistical analyses were performed using the R Statistical Package (v4.2.1; R Core Team 2022, Vienna, Austria) embedded within PearlDiver.

### Results

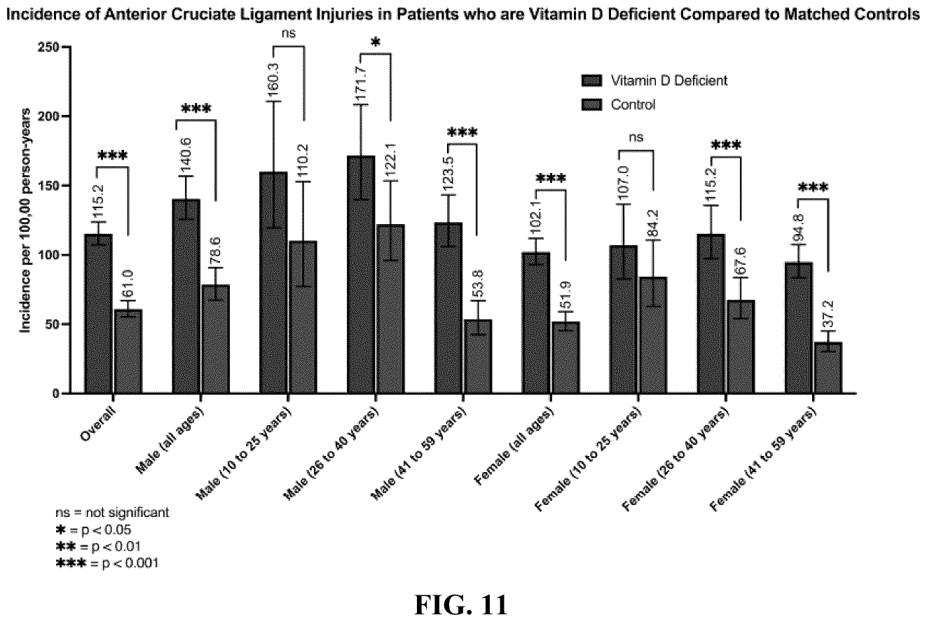

The mean age of each cohort was 41.9±12.6 years, with a female predominance at 65.8%. The mean Charlson comorbidity index was 0.3±0.7 and the percentage of patients with a history of tobacco use (23.5%), diabetes mellitus (24.1%), or a body mass index ≥25 kg/m² (31.6%) within each cohort was statistically similar (p=1.000 for all). The overall incidence of ACL tears over the two-year period following a diagnosis of vitamin D deficiency was 115.2 per 100,000 person-years (95% CI, 107.2-123.7), compared to 61.0 (95% CI, 55.2-67.2) in the matched control cohort (FIG. 11).

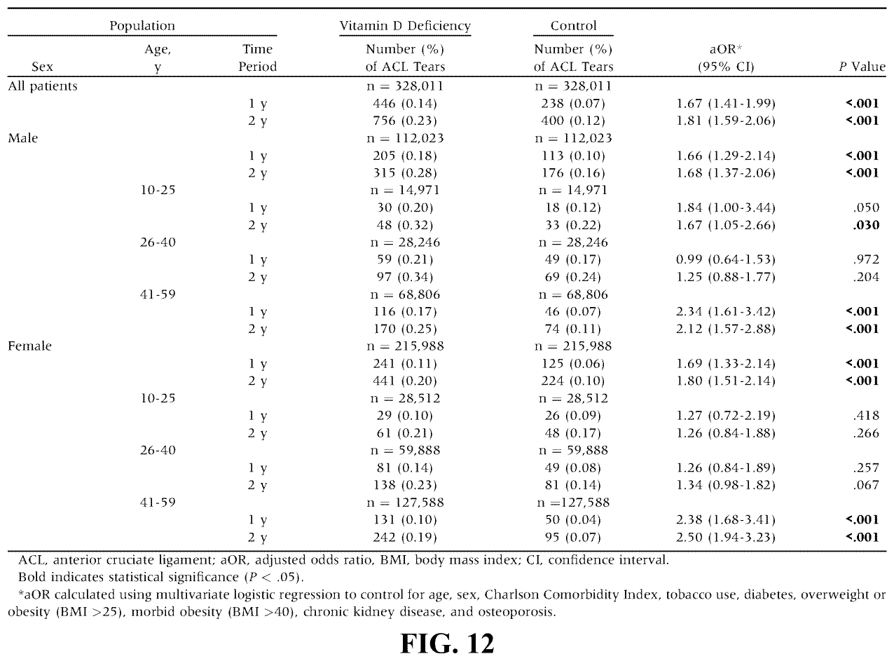

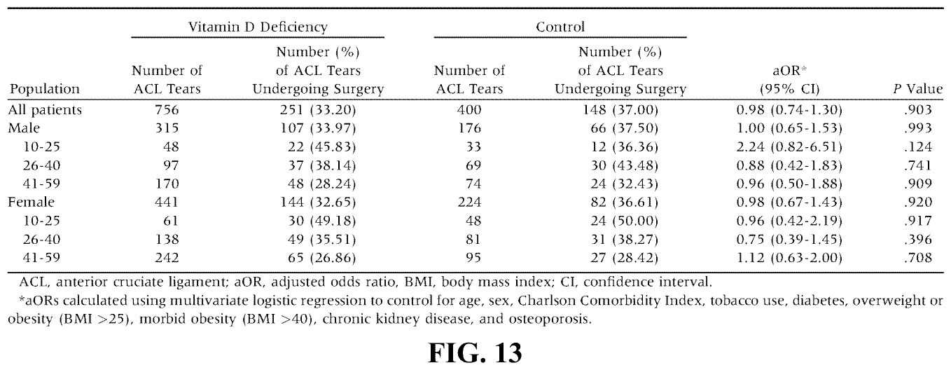

Patients aged 10 to 59 diagnosed with hypovitaminosis D had a significantly increased likelihood of suffering from an ACL tear over both a one- (aOR=1.67, 95% CI, 1.41-1.99) and two-year (aOR=1.81, 95% CI, 1.59-2.06) period (p<0.001 for both). This trend was also present in the sex-specific cohorts (see table in FIG. 12). Patients aged 41 to 59 years in both the male and female cohorts had the greatest increased odds compared to their respective controls, with all odds ratios greater than 2.00 for both the one- and two-year time points (p<0.001 for all). Dissimilarly, no difference was observed in the rates of primary tears in males aged 10 to 25 at 1 year, males aged 26 to 40 at both time points, females aged 10 to 25 at both time points, and females aged 26 to 40 at both time points. There was also no difference in the rates of undergoing primary ACL reconstruction between the vitamin D-deficient and control cohorts (see table in FIG. 13, p>0.05 for all).

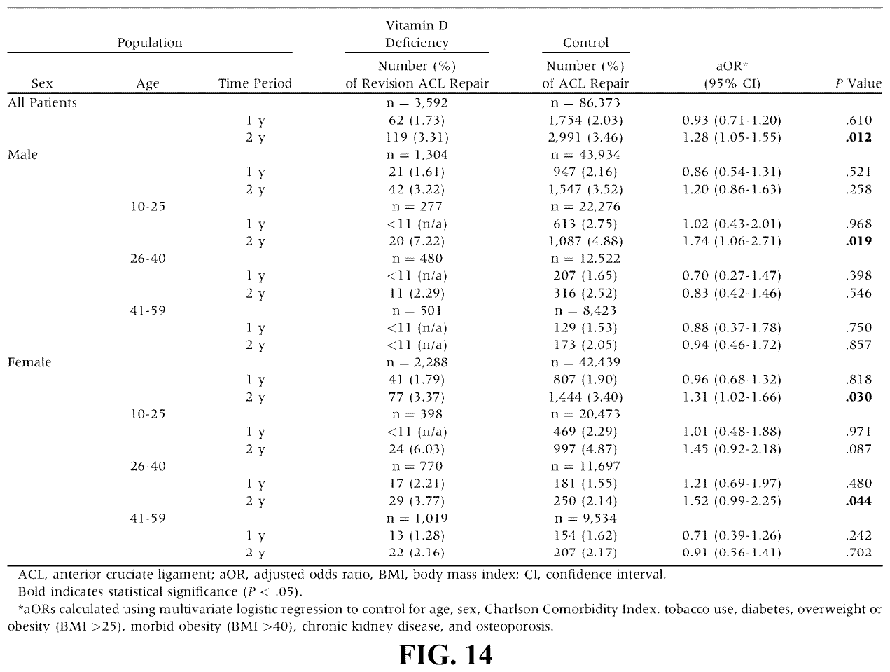

In the second analysis of patients undergoing ACL reconstruction, there was no difference in the rates of revision ACL reconstruction within one year of a primary ACL reconstruction. At the two-year mark, vitamin D-deficient patients were significantly more likely to undergo revision ACL reconstruction (aOR=1.28, 95% CI, 1.05-1.55, p=0.012). This trend remained for three of the sex- and age-specific cohorts as well: males aged 10 to 25, all females, and females aged 26 to 40 (see table in FIG. 14).

### Discussion

The main findings of this study were that patients diagnosed with vitamin D deficiency experience significantly increased rates of primary ACL tears and revision ACLR when compared to a control. Vitamin D deficiency is a global public health issue with profound impacts on a spectrum of health conditions.⁶⁵ However, given the lack of universal vitamin D deficiency screening guidelines in adolescent and adult populations within the United States, the exact rate of vitamin D deficiency is not known in the control cohort as they were either not screened or screened once and had a normal value, but sequentially developed a deficiency that was not captured.⁴²

In orthopaedics, vitamin D deficiency is associated with increased postoperative complications following knee arthroplasty, hip arthroplasty, and rotator cuff repair.⁸⁸ Additionally, studies have shown that vitamin D deficiency impairs normal musculoskeletal biology in vivo and in vitro.^{31,88,05,34} However, the clinical manifestations of these effects in primary orthopaedic injuries have not been well characterized. Regarding ACL tears specifically, given the prevalence and substantial burden placed on patients by these injuries, it is imperative to better understand the potentially modifiable risk factors for primary ACL injury.¹ The present findings identify an association between being diagnosed with vitamin D deficiency, a widely prevalent and easily treatable medical comorbidity, and increased rates of ACL tears and the need for revision ACL reconstruction after a primary procedure among certain age groups. While the present study reports the association between vitamin D deficiency and increased risk, there is currently no high-quality data suggesting that treatment of the deficiency modifies the risk.

Prior studies have demonstrated the role of poor knee mechanics during dynamic movements that strain the ACL and increase the risk of injury.^{111,14,126,27} In order to maintain alignment during activity, sufficient strength in Type II muscle fibers of the lower limb is necessary.^{12,30,96,10} Notably, these type II muscle fibers are directly influenced by the presence of vitamin D as it serves to facilitate muscle cell growth, differentiation, and contraction by stimulating the expression of growth factors.^{45,49,78} The lack of these anabolic signals leads to uncontrolled activity of degradative proteolytic pathways, leading to type II muscle atrophy and weakness with both eccentric and concentric contraction patterns.^{7,8,13,18,71,102,108} This motor weakness not only increases one's risk for initial rupture but may also have significant ramifications for postoperative recovery and risk of re-rupture.^{7,9,18,31,102,108} Kyritsis et al. found that a 10% deficit in knee flexor to extensor strength ratio was associated with a 10.6 fold-increased risk of ACL re-injury upon return to sport.⁸⁰ In a 2011 study by Barker et al., patients with low vitamin D levels prior to and after surgery had lower single-leg peak isometric forces than patients with vitamin D levels ≥30 ng/mL.⁹ These findings suggest that vitamin D deficiency may affect both primary injury and postoperative recovery, which support the results from the present study. Nonetheless, there are also several studies identifying vitamin D deficiency in high-level athletes, demonstrating the ability to perform at a high physical level with hypovitaminosis, suggesting the relationship between vitamin D deficiency and muscle fiber function and integrity is complex and multifactorial.^{27,67}

In addition to its effects on skeletal muscle, vitamin D deficiency alters the local synovial environment in which the ACL resides.^{31,82,16,36} Vitamin D-deficient joints display an increase in inflammatory cytokines and matrix metalloproteinases (MMPs), a class of proteins involved in degradation of the soft tissues and cartilage in the knee.^{31,62,82,99,16} Studies have shown that inflammatory mediators such as tumor necrosis factor-α (TNF-α) and nuclear factor-κB (NF-κB) initiate cellular changes in the ACL through the activation of MMPs.^{44,58,70,90,99} In particular, imbalanced MMP activity has been implicated in promoting excess connective tissue degradation and pathogenic extracellular matrix remodeling.⁹⁴ As no published literature specifically implicates vitamin D deficiency as a cause of structural changes, including collagen fiber orientation and impairing the elasticity and strength of the ACL, this is a speculative explanation to account for the results of the present study.

The findings in the present study suggest that patients diagnosed with vitamin D deficiency have increased rates of revision ACLR. Although the success of ACLR is dependent upon numerous factors, stability at the bone-graft interface is of critical importance. This process and its relationship to vitamin D have been thoroughly studied with rotator cuff repairs. Harada et al. found that vitamin D-deficient patients were 54% more likely to require revision surgery for rotator cuff repair and suffered greater postoperative complications than patients without vitamin D deficiency.⁵⁷ Robertson et al. found elevated levels of MMPs to be highly associated with degradation of the repaired tendon and subsequent rotator cuff repair failure in humans.¹¹⁷ In an animal model of rotator cuff repair, Angeline et al. found that vitamin D-deficient rats had decreased fibrocartilage formation and poor collagen organization.⁵ This translates to a potentially weaker tendon-to-bone incorporation and higher rates of rotator cuff repair failure.³¹ In the limited ACL-specific literature available, Barker et al. found that inflammatory cytokines were elevated following ACLR in vitamin D-deficient patients.⁹ Taken collectively, with upregulation of inflammatory cytokines and MMPs as a result of vitamin D deficiency, ACL grafts may be subject to similar breakdown and potentially contribute to higher rates of revision ACLR, though it is not possible to fully extrapolate the rotator cuff repair animal model to the human ACL.^{82,33}

Fortunately, vitamin D deficiency is a readily modifiable risk factor with treatments that have proven effects on musculoskeletal health. In a meta-analysis, Stockton et al. found that supplementation with vitamin D in deficient adults results in a large effect size increase in hip muscle strength.¹³¹ These results are further corroborated by a randomized-controlled trial demonstrating that vitamin D-deficient athletes experience improvements in muscle strength and performance after supplementation.²³ Studies have also reported that vitamin D supplementation also decreases circulating MMP-9 levels in deficient patients.^{4,133} Regarding the role of vitamin D supplementation in this population, this is an interesting topic of future research as there are currently no high-quality studies that associate the treatment of vitamin D deficiency with decreased rates of ACL injury when compared to those with untreated vitamin D deficiency. Overall, the results of this study suggest that there may be an association between vitamin D deficiency and the risk of ACL injury. Further study is needed to validate this finding.

Limitations: This study is not without limitations. Inherent to any retrospective database analysis, the results depend on accurate diagnostic coding of injuries. Similarly, the diagnosis of vitamin D deficiency requires clinical suspicion of the condition and subsequent testing, which may result in missed diagnoses. However, due to the large number of patients included in the database with a diagnosis of vitamin D deficiency, this limitation should not greatly influence the interpretation of the results and conclusions of this study. Second, as previously described, there is no way to quantify the exact rate of vitamin D deficiency in the control cohort as many patients go undiagnosed. Third, while several factors were included in the matching and multivariable regression model to control for potential confounding variables, there are several other variables that contribute to the development of an ACL tear and primary and secondary reconstruction. For example, this database does not include data on patient activity level, compliance with the postoperative recovery plan, surgical technique, preoperative knee laxity, and more. Similarly, this database does not provide the exact vitamin D level in these patients with diagnosed vitamin D deficiency. Hence, this study is unable to correlate the degree of vitamin D deficiency with rates of ACL tears or primary and secondary surgery. Lastly, while these results are representative of a large cohort, they were generated from insurance claims from a single insurance provider and thus may not be a representative sample of patients with Medicare, Medicaid, or those without insurance.

### Vitamin D Study Conclusions

This study reports an association between patients previously diagnosed with hypovitaminosis D and significantly increased rates of both index ACL tears (81% increase within two years) and revision ACLR (28% within two years). These results identify a population with increased odds of injury and provide valuable knowledge as we expand our understanding of the relationship between vitamin D and musculoskeletal health. In some embodiments, the present innovation proposes synergistic effects while administering an antagonist of relaxin-2 and/or an antagonist of a receptor of relaxin 2, along with a vitamin D treatment or regimen, to the subject, or a precursor of said antagonist or vitamin, whereby one or more connective tissue characteristics are modulated/changed in the subject such as to prevent and/or treat the connective tissue injury.

The present innovation, in one of its broadest embodiments, provides a method for preventing and/or treating a connective tissue injury characteristic, the method comprising the steps of: (1) providing a subject; and (2) administering an antagonist of relaxin-2 and/or an antagonist of a receptor of relaxin-2 to the subject (optionally along with administration of vitamin D, as discussed above), whereby one or more connective tissue characteristics are modulated/changed in the subject such as to prevent and/or treat the connective tissue injury characteristic in the subject. In some embodiments, the method above can be carried out by any use of a precursor (or a prodrug) of the antagonist. The subject can be instructed to self-administer an antagonist, for example by utilizing a supplement, a natural product, or a dietary change. The method disclosed herein can be, in some embodiments, wherein the connective tissue comprises a ligament, and the antagonist comprises folic acid, NADH, or a combination thereof.

In some embodiments, the method disclosed above can be wherein the subject has previously undergone a surgical reconstruction, and/or can be a normal healthy subject, lacking a pre-diagnosed connective tissue disease or condition, and step (2) comprises in part a prophylactic method used to prevent a connective tissue injury/rupture. In some embodiments, the methods disclosed above can be wherein the subject is expected to or likely to participate in an activity, a sport, or a movement in the future that can increase the chances of a connective tissue injury occurring in the subject.

According to some aspects, the method disclosed above can be wherein the subject is at a risk of or in need of a connective tissue care, having a diagnosed connective tissue injury characteristic, or has been determined to be at a risk of a connective tissue injury by a health care provider. According to some aspects, the method disclosed above is wherein the subject is suspected of having or has been diagnosed by a health care provider as having, a disorder including an autoimmune disease of connective tissue, an undifferentiated connective tissue disorder, a myxomatous degeneration, a congenital disease, a neoplasm, or a combination thereof.

In some embodiments, a method for cultivating a connective tissue in vitro is provided, the method comprising the step of contacting the cultured connective tissue with an antagonist of relaxin-2 and/or an antagonist of a receptor of relaxin-2, and/or vitamin D, before, during, or after said cultivating, whereby one or more connective tissue characteristics are modulated/changed. In some embodiments, the method of cultivating can be wherein the connective tissue is suitable for implantation into a subject in need thereof. In some embodiments, this method can be wherein the connective tissue is a cell generated tissue graft and/or wherein the connective tissue cultivated in vitro is derived from or originates from a subject undergoing, or planned to undergo, a ligament reconstruction surgery.

The method of any preceding paragraph, in some embodiments, can be wherein the connective tissue includes ligament, tendon, cartilage, intervertebral disc, cornea, or a combination thereof. The connective tissue can comprise a fibrillar collagen or a collagen. For example, the collagen can include type I (or IA), II, III (or IIIA), V, XI, or a combination thereof.

The methods disclosed above, in some embodiments, can be wherein the antagonist comprises a therapeutic agent, a supplement, a formulation, a natural product, a drug, a biologic, an aerosol, a liquid, a particle, microparticle, or nanoparticle, a biologic, or a combination thereof. In an example, the antagonist includes a daily dose of about 400 micrograms/day antagonist for a healthy human subject, based on general daily recommended values.

In some embodiments, the methods disclosed above can be wherein the method is long-term. The antagonists can be, for example, a blocker, an orthosteric antagonist, allosteric antagonist, conformation changing binder, a modulator, a partial inverse agonist/inverse agonist, or a combination thereof.

The method of any preceding paragraph above, in some embodiments, can be wherein the method is in the form of instructions provided in a kit, said kit optionally including a formulation and/or therapeutic agent operative to block/antagonize relaxin-2 and/or a block/antagonize a receptor of relaxin-2.

In some embodiments, a method for diagnosing and/or determining a risk or a likelihood of a connective tissue injury/defect in a subject is disclosed herein, the method comprising quantifying the number of RXFP2 positive cells in a connective tissue derived from the subject and comparing the number to RXFP2 positive cells to a standard determination.

According to some aspects, a method for designing, screening, and/or locating a therapeutic agent, said therapeutic agent suitable for preventing and/or treating a connective tissue injury characteristic in a subject in need thereof is demonstrated herein, the method comprising the steps of: (1) providing a candidate therapeutic agent suspected of having activity including antagonism of relaxin-2 and/or antagonism of a receptor of relaxin-2; and (2) screening the candidate therapeutic agent such as to determine/measure antagonism of relaxin-2 and/or a receptor of relaxin-2.

The screening method can be wherein the method is repeated for a plurality of candidate therapeutic agents and a measurement/determination of antagonism of relaxin-2 and/or a receptor of relaxin-2 is provided for each of the candidate therapeutic agents. Each of the candidate therapeutic agents can be ranked in comparison to the other each of the candidate therapeutic agents for an efficacy of relaxin-2 and/or a receptor of relaxin-2.

The methods described above can, in some embodiments, include wherein an expression/activity of MMP1 and/or MMP13 is decreased by the method.

In all aspects, our invention of the field of use for Folic Acid and NADH to block relaxin-2 and its receptors, thereby augmenting ligament structure preventing ligament mechanical failure or injury, provides a novel discovery. This discovery is novel considering a field of work that shows the deleterious effects of relaxin-2 on ligament biology and its responsiveness to the agonist of estrogen. While some improvement in ACL injury prevention has been show with oral contraceptive medicines, this treatment is not always possible or desirable in young athletes. Our discovery provides a treatment option to help prevent ligamentous injury. This medicine/supplement would have appeal to athletes at high risk for injuries such as ACL tear or individuals who suffer from ligamentous hyperlaxity. For example, serum relaxin levels are equivalent in young men and young women.¹⁴⁰ Serum relaxin concentration was elevated in subjects prior to experiencing shoulder instability Owens 2016.¹⁰⁰ As shown in the Examples below, we have isolated the relaxin receptor in the shoulder labrum and capsule in patients with shoulder instability undergoing surgery. We have isolated the relaxin receptor in the ACL tissue of patients undergoing ACL reconstruction as well as elucidated gender differences of ACL and relaxin. We have examined known relaxin and relaxin receptor blockers (e.g., Example 1 below), and then performed in vitro analysis on the previously stated blockers showing the data.

In some embodiments, the techniques described herein relate to a method for preventing and/or treating a connective tissue injury characteristic, the method including the steps of: (1) providing a subject; and (2) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury.

According to some aspects, the techniques described herein relate to a method, wherein the connective tissue includes a ligament, and the antagonist includes folic acid, NADH, or a combination thereof.

In some embodiments, the techniques described herein relate to a method, wherein the subject is a subject that has previously undergone a surgical reconstruction, and/or a normal healthy subject, lacking a pre-diagnosed connective tissue disease or condition, and step (2) includes in part a prophylactic method used to prevent a connective tissue injury and/or rupture.

In some embodiments, the techniques described herein relate to a method, wherein the subject is expected to or likely to participate in an activity, a sport, or a movement in the future that can increase the chances of a connective tissue injury occurring in the subject.

According to some aspects, the techniques described herein relate to a method, wherein the subject is at a risk of or in need of a connective tissue care, having a diagnosed connective tissue injury characteristic, or has been determined to be at a risk of a connective tissue injury by a health care provider.

In some embodiments, the techniques described herein relate to a method, wherein the subject is suspected of having, or has been diagnosed by a health care provider as having, a disorder including an autoimmune disease of connective tissue, an undifferentiated connective tissue disorder, a myxomatous degeneration, a congenital disease, a neoplasm, or a combination thereof.

In some embodiments, the techniques described herein relate to a method for cultivating a connective tissue in vitro, the method including the step of contacting the culturing connective tissue with an antagonist of relaxin-2 and/or an antagonist of a receptor of relaxin-2, or a precursor of said antagonist, before, during, or after said cultivating, whereby one or more connective tissue characteristics are modulated/changed.

According to some aspects, the techniques described herein relate to a method, wherein the connective tissue is suitable for implantation into a subject in need thereof.

In some embodiments, the techniques described herein relate to a method, wherein the connective tissue is a cell generated tissue graft.

In some embodiments, the techniques described herein relate to a method, wherein the connective tissue cultivated in vitro is derived from or originates from a subject undergoing, or planned to undergo, a ligament reconstruction surgery.

According to some aspects, the techniques described herein relate to a method, wherein the connective tissue includes ligament, tendon, cartilage, intervertebral disc, cornea, or a combination thereof.

In some embodiments, the techniques described herein relate to a method, wherein the connective tissue includes a ligament.

In some embodiments, the techniques described herein relate to a method, wherein the connective tissue includes a fibrillar collagen or a collagen.

According to some aspects, the techniques described herein relate to a method, wherein the collagen includes type I (or IA), II, III (or IIIA), V, XI, or a combination thereof.

In some embodiments, the techniques described herein relate to a method, wherein the antagonist includes a therapeutic agent, a supplement, a formulation, a natural product, a drug, a biologic, an aerosol, a liquid, a particle, microparticle, or nanoparticle, a biologic, or a combination thereof.

In some embodiments, the techniques described herein relate to a method, wherein the antagonist includes a daily dose of about 400 micrograms/day antagonist for a healthy human subject. In some embodiments, a daily dose can be in a range from about 50 micrograms/day to about 1 gram per day, in a range from about 100 micrograms/day to about 500 mg per day, in a range from about 200 micrograms/day to about 100 mg per day, or in a range from about 300 micrograms/day to about 50 mg per day, depending on the subject.

According to some aspects, the techniques described herein relate to a method, wherein the method is long-term.

In some embodiments, the techniques described herein relate to a method, wherein the antagonist is a blocker, an orthosteric antagonist, allosteric antagonist, conformation changing binder, a modulator, a partial inverse agonist/inverse agonist, or a combination thereof.

In some embodiments, the techniques described herein relate to a method, wherein the method is in the form of instructions provided in a kit, said kit optionally including a formulation and/or therapeutic agent operative to block/antagonize relaxin-2 and/or a block/antagonize a receptor of relaxin-2.

According to some aspects, the techniques described herein relate to a method, wherein the antagonist includes a relaxin antagonist/relaxin receptor antagonist from examples in Table 2 and/or Table 3, or from a library of known therapeutic agents.

In some embodiments, the techniques described herein relate to a method for diagnosing and/or determining a risk or a likelihood of a connective tissue injury/defect in a subject, the method including quantifying the number of RXFP2 positive cells in a connective tissue derived from the subject and comparing the number to RXFP2 positive cells to a standard determination.

In some embodiments, the techniques described herein relate to a method for designing, screening, and/or locating a therapeutic agent, said therapeutic agent suitable for preventing and/or treating a connective tissue injury characteristic in a subject in need thereof, the method including the steps of: (1) providing a candidate therapeutic agent, or a precursor of the candidate, suspected of having activity including antagonism of relaxin-2 and/or antagonism of a receptor of relaxin-2; and (2) screening the candidate therapeutic agent or precursor such as to determine/measure antagonism of relaxin-2 and/or a receptor of relaxin-2, wherein said antagonism is determinative of the suitability for prevention of an injury.

According to some aspects, the techniques described herein relate to a method, wherein the method is repeated for a plurality of candidate therapeutic agents and a measurement/determination of antagonism of relaxin-2 and/or a receptor of relaxin-2 is provided for each of the candidate therapeutic agents.

In some embodiments, the techniques described herein relate to a method, wherein each of the candidate therapeutic agents is ranked in comparison to the other each of the candidate therapeutic agents for an efficacy of relaxin-2 and/or a receptor of relaxin-2.

In some embodiments, the techniques described herein relate to a method for preventing and/or treating a connective tissue injury characteristic, the method including the steps of: (1) providing a subject; and (2) administering vitamin D (D1, D2, D3, D4, D5, or an analogue thereof) to the subject, or a precursor of said vitamin D, whereby one or more connective tissue characteristics are modulated/changed in the subject such as to prevent and/or treat the connective tissue injury.

According to some aspects, the techniques described herein relate to a method for cultivating a connective tissue in vitro, the method including the step of contacting the culturing connective tissue with vitamin D (D1, D2, D3, D4, D5, or an analogue thereof), or a precursor of said vitamin D, whereby: (1) one or more connective tissue characteristics are modulated/changed; and (2) one or more differences in effects of the modulated/changed are observed between cells derived from a female subject and cells derived from a non-female or a male subject.

In some aspects, the techniques described herein relate to a method or a feature, wherein the vitamin D includes vitamin D3 (cholecalciferol), vitamin D2 (ergocalciferol), or a combination thereof.

In some embodiments, the techniques described herein relate to a method, wherein the analogue includes Alfacalcidol, Calcipotriol (calcipotriene), Doxercalciferol, Falecalcitriol, Paricalcitol, Tacalcitol, or a combination thereof.

According to some aspects, the techniques described herein relate to a method, wherein the method is used in any combination with the method, or before or after any one.

In some aspects, the techniques described herein relate to a method, wherein Type I collagen expression is observably increased after vitamin D in female cells compared to such observable expression under the same conditions in male cells.

In some embodiments, the techniques described herein relate to a method, wherein an expression/activity of MMP1 and/or MMP13 is decreased by the method.

According to some aspects, the techniques described herein relate to a method, wherein MMP13 gene expression is different between cells derived from a female subject and cells derived from a male subject after one or more vitamin D treatments.

In some aspects, the techniques described herein relate to a method, wherein MMP13 gene expression is reduced after one or more vitamin D treatments at about 10 nmol/L in female cells and at about 100 nmol/L in male cells.

In some embodiments, the techniques described herein relate to a method for preventing and/or treating a connective tissue injury characteristic, the method including the steps of: (1) providing a subject; (2) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury; and (3) administering vitamin D (D1, D2, D3, D4, D5, or an analogue thereof) to the subject, or a precursor of said vitamin D, whereby one or more connective tissue characteristics are modulated/changed in the subject such as to prevent and/or treat the connective tissue injury; wherein an execution of steps (2) and (3) is separated by a time period of less than about 1 year.

In some embodiments, the techniques described herein relate to a method for modulating biological characteristics, including: administering a therapeutic agent to a biological entity; modulating biological characteristics in the biological entity; preventing and/or treating a biological condition in the biological entity.

According to some aspects, the techniques described herein relate to a method, wherein the therapeutic agent is an antagonist of relaxin-2, which is a hormone involved in the regulation of the reproductive system and has been implicated in the modulation of connective tissue flexibility.

In some embodiments, the techniques described herein relate to a method, wherein the therapeutic agent is an antagonist of a receptor of relaxin-2, specifically targeting the RXFP1 receptor which is known to mediate the physiological actions of relaxin-2.

According to some aspects, the techniques described herein relate to a method, wherein the therapeutic agent is a precursor of an antagonist of relaxin-2 or a receptor of relaxin-2, wherein the precursor is metabolized in the biological entity to form the active antagonist that exerts the therapeutic effects.

In some embodiments, the techniques described herein relate to a method, wherein the biological characteristics are one or more connective tissue characteristics, including but not limited to collagen composition, tensile strength, and elasticity of the connective tissue.

According to some aspects, the techniques described herein relate to a method, wherein the biological condition is a connective tissue injury, such as a sprain, strain, tear, or other form of damage to the connective tissue that supports, connects, or separates different types of tissues and organs in the body.

In some embodiments, the techniques described herein relate to a method, wherein the biological entity is a subject, and the subject may be a human or an animal, wherein the method is applied in a clinical or veterinary setting for the improvement of the subject's health condition.

According to some aspects, the techniques described herein relate to a method, wherein the modulation of biological characteristics results in a change in the biological characteristics, such as an increase or decrease in the production of collagen, alterations in the cross-linking of connective tissue fibers, or changes in the biomechanical properties of the tissue.

In some embodiments, the techniques described herein relate to a method, wherein the prevention and/or treatment of the biological condition results in a change in the biological condition, leading to an improvement in symptoms, a reduction in pain, inflammation, or other clinical signs associated with the connective tissue injury, or a restoration of the normal function of the affected tissue.

According to some aspects, the techniques described herein relate to a method for preventing and/or treating a connective tissue injury in a subject, the method including the step of administering vitamin D (D1, D2, D3, D4, D5, or an analogue thereof) to the subject, or a precursor of said vitamin D, whereby one or more connective tissue characteristics are modulated/changed in the subject such as to prevent and/or treat the connective tissue injury.

In some embodiments, the techniques described herein relate to a method, wherein the vitamin D includes vitamin D3 (cholecalciferol), vitamin D2 (ergocalciferol), or a combination thereof.

According to some aspects, the techniques described herein relate to a method, wherein the analogue includes Alfacalcidol, Calcipotriol (calcipotriene), Doxercalciferol, Falecalcitriol, Paricalcitol, Tacalcitol, or a combination thereof.

In some embodiments, the techniques described herein relate to a method, wherein the therapeutic agent is administered in a dosage form selected from the group consisting of a tablet, a capsule, a solution, an injection, and a transdermal patch.

According to some aspects, the techniques described herein relate to a method, wherein the administration of the therapeutic agent is followed by a monitoring step, wherein the biological characteristics of the biological entity are monitored to assess the efficacy of the treatment.

In some embodiments, the techniques described herein relate to a method, wherein the therapeutic agent is administered in conjunction with one or more additional therapeutic agents that contribute to the modulation of the biological characteristics.

According to some aspects, a step can involve administering a therapeutic agent to a subject with the goal of modulating connective tissue characteristics to prevent or treat connective tissue injury. The therapeutic agent targets either relaxin-2 or its receptor, or it may be a precursor that is metabolized into the active form within the subject's body. This step is the primary action in the method for addressing connective tissue injuries.

Regarding the nature of the therapeutic agent, it can be an antagonist of relaxin-2, an antagonist of a receptor of relaxin-2, or a precursor that becomes the active antagonist. The interaction between the therapeutic agent and the target inhibits the normal activity that affects connective tissue properties, such as collagen composition, tensile strength, and elasticity.

In general, the therapeutic agent can be delivered in a specific form, which could include tablets, capsules, solutions, injections, or transdermal patches. This ensures that the agent is delivered in a manner suitable for the subject's condition.

The subject can be monitored after the administration of the therapeutic agent. This involves assessing the treatment's efficacy by observing changes in the connective tissue properties. Various diagnostic tools and methods may be used to measure these properties.

The therapeutic agent may be administered alongside additional agents. This is based on the understanding that a combination of agents may have a synergistic effect or address multiple pathways involved in connective tissue modulation and injury treatment.

The technology encompasses the selection and administration of a specific therapeutic agent or its precursor, the method of delivery, and the subsequent observation of its effects on connective tissue properties, all aimed at addressing connective tissue injuries in a subject.

The technology involves the process of altering characteristics within a subject that has experienced or is at risk of a connective tissue injury. This targets the modulation of characteristics such as collagen composition, tensile strength, and elasticity of connective tissues, which are essential for the structural integrity and function of ligaments, tendons, and cartilage.

The modulation is achieved through the administration of a specific agent that interacts with the hormone relaxin-2 or its receptor. Relaxin-2 is involved in the regulation of connective tissue flexibility, and by antagonizing its action or the action of its receptor, the method aims to induce changes in the connective tissue to address the injury or the risk thereof.

The characteristics that are modulated, can include the composition, strength, and elasticity of the connective tissue. The modulation results in a tangible change in these characteristics, which could involve alterations in collagen production or the cross-linking of connective tissue fibers, affecting the tissue's biomechanical properties. The nature of the modulation would depend on the specific condition being addressed and the desired outcome.

The process of modulation involves the interaction of the administered agent with cellular receptors, signaling pathways, and the extracellular matrix of the connective tissue. The agent would be administered in a suitable form and potentially in conjunction with other agents to achieve the desired effect. Monitoring may follow to assess the effectiveness of the treatment.

In summary, herein is described the action of altering connective tissue characteristics through the administration of specific agents, with the goal of addressing connective tissue injuries. This involves an understanding of the biological mechanisms and the careful selection and administration of the appropriate agents.

The data provided herein clearly shows a synergistic effect can be achieved and a risk of injury reduction can be achieved, for example, for female athletes.

The methods disclosed herein can be carried out by use of instructions provided to a subject, for example, on a label, electronic instructions, via telecommunication, or instructions provided by demonstration (e.g., video). The methods herein can be applied prophylactically to a normal healthy subject. A subject can be one who has been previously diagnosed with or identified as suffering from or having a condition in need of treatment (e.g., a genetic disorder) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, or at risk of developing that condition.

### EXAMPLES

The invention now being generally described, it will be more readily understood by reference to the following examples which are included merely for purposes of illustration of certain aspects and embodiments of the present invention and are not intended to limit the invention.

### Example 1. Virtual Screening of FDA-Approved Drug Library

Preparation of the Protein Structure: The 3D structure of human relaxin-2 (PDB ID: 2MV1) and RXFP2 LDLa module (2M96) were retrieved from the RCSB Protein Data Bank (rcsb.org). The structure was optimized using Schrödinger Maestro with default parameters. The protein structure was prepared by adding missing atoms, assigning bond orders, and optimizing hydrogen positions using Maestro's Protein Preparation Wizard. Corrections or modifications, such as removing water molecules or unwanted ligands, were performed as necessary.

Identification of the Receptor: After minimization, the protein structure was visualized to identify potential binding sites based on structural features like cavities, clefts, or pockets. Regions capable of accommodating ligands or small molecules were identified. A molecular surface representation of the protein was generated using Maestro's surface panel to visualize its topology and facilitate the identification of potential binding sites. The surface was further analyzed for properties such as electrostatic potential or hydrophobicity to identify regions involved in ligand binding. Conserved residues within the protein structure, indicative of their importance for protein function, were identified. Sequence alignment tools were used to identify these conserved residues. The chemical properties, spatial arrangement, and potential interactions of the identified residues within the binding site were analyzed. All these analyses were performed using default parameters.

Generation of the Grid: In Maestro, the Grid Generation panel was used to define the grid parameters, including size, center, and spacing. These parameters were set to cover the entire active site while minimizing unnecessary computational resources.

Ligand Preparation: A total of 2454 FDA-approved drugs were retrieved from the BindingDB (bindingdb.org) and used as ligands in the study. The structures of the ligands were further optimized using Maestro's LigPrep module. LigPrep generated low-energy conformations, ionization states, and tautomers of the ligands to ensure their suitability for docking and other molecular modeling techniques.

Molecular Docking: Molecular docking was performed using Maestro's Glide module. The OPLS3e force field, default parameters, and search algorithms were employed. The ligands, after LigPrep, were docked into the active site using the generated grid.

The docking results were analyzed to identify potential binding poses and interactions. From the top 30 candidates obtained through virtual screening, based on Glide scores, ligands were selected for wet lab testing. Examples of docking results are presented in Table 2 (Relaxin Receptor Antagonists) and in Table 3 (Relaxin Receptor Antagonists).

Docking score is the scoring function used to predict the binding affinity of both ligand and target once it is docked. The drugs with high docking score and low toxicity are selected. Folic acid is found as a relaxin-2 antagonist, and it is a dietary supplement which helps with ligament, hair, skin, and bone health. Another option is a relaxin-2 receptor antagonist nicotinamide adenine dinucleotide hydride (NADH), a food supplement that has been shown to support ligament, joint, and cartilage health.

While Folic acid was selected, any of the compounds showing activity could be claimed in certain aspects of the technology disclosed herein.

TABLE 2
Example Relaxin Receptor Antagonists and Docking Scores.

| Entry ID | GENERIC NAME | docking score |
|---|---|---|
| 1 | not used | not tested |
| 2 | Ceruletide | −10.341 |
| 3 | Desmopressin | −8.284 |
| 4 | Edotreotide gallium Ga-68 | −8.193 |
| 5 | Bleomycin | −8.073 |
| 6 | Edotreotide gallium Ga-68 | −8.072 |
| 7 | Bleomycin | −7.932 |
| 8 | Bleomycin | −7.851 |
| 9 | Leuprolide | −7.692 |
| 10 | Bleomycin | −7.474 |
| 11 | Bleomycin | −7.358 |
| 12 | Edotreotide gallium Ga-68 | −7.235 |
| 13 | Polymyxin B | −7.11 |
| 14 | Bleomycin | −7.081 |
| 15 | Bleomycin | −7.078 |
| 16 | Bleomycin | −6.999 |
| 17 | Bleomycin | −6.978 |
| 18 | Acarbose | −6.836 |
| 19 | Polymyxin B | −6.79 |
| 20 | Edotreotide gallium Ga-68 | −6.673 |
| 21 | Bleomycin | −6.631 |
| 22 | Edotreotide gallium Ga-68 | −6.623 |
| 23 | Pentagastrin | −6.524 |
| 24 | Edotreotide gallium Ga-68 | −6.513 |
| 25 | Edotreotide gallium Ga-68 | −6.505 |
| 26 | Acarbose | −6.493 |
| 27 | Bleomycin | −6.457 |
| 28 | Bleomycin | −6.433 |
| 29 | Cetrorelix | −6.427 |
| 30 | Bleomycin | −6.427 |
| 31 | Bleomycin | −6.411 |
| 32 | Edotreotide gallium Ga-68 | −6.401 |
| 33 | Bleomycin | −6.324 |
| 34 | Bleomycin | −6.272 |
| 35 | Bleomycin | −6.272 |
| 36 | Abarelix | −6.238 |
| 37 | Bleomycin | −6.227 |
| 38 | Bleomycin | −6.225 |
| 39 | Bleomycin | −6.204 |
| 40 | Bleomycin | −6.155 |
| 41 | Bleomycin | −6.149 |
| 42 | Bleomycin | −6.12 |
| 43 | Goserelin | −6.107 |
| 44 | Bleomycin | −6.064 |
| 45 | Bleomycin | −6.05 |
| 46 | Bleomycin | −6.026 |
| 47 | Bleomycin | −6.016 |
| 48 | Edotreotide gallium Ga-68 | −5.973 |
| 49 | Bleomycin | −5.956 |
| 50 | Bleomycin | −5.952 |
| 51 | Bleomycin | −5.912 |
| 52 | Bleomycin | −5.896 |
| 53 | Bleomycin | −5.887 |
| 54 | Bleomycin | −5.847 |
| 55 | Bleomycin | −5.78 |
| 56 | Bleomycin | −5.741 |
| 57 | Bleomycin | −5.727 |
| 58 | Bleomycin | −5.722 |
| 59 | Octreotide | −5.722 |
| 60 | Bleomycin | −5.704 |
| 61 | Edotreotide gallium Ga-68 | −5.681 |
| 62 | Bleomycin | −5.655 |
| 63 | Bleomycin | −5.643 |
| 64 | Bleomycin | −5.639 |
| 65 | Bleomycin | −5.624 |
| 66 | Bleomycin | −5.615 |
| 67 | Droperidol | −5.598 |
| 68 | Bleomycin | −5.579 |
| 69 | Travoprost | −5.566 |
| 70 | Bleomycin | −5.565 |
| 71 | Bleomycin | −5.551 |
| 72 | Leuprolide | −5.521 |
| 73 | Bleomycin | −5.511 |
| 74 | Bleomycin | −5.465 |
| 75 | Bleomycin | −5.447 |
| 76 | Bleomycin | −5.407 |
| 77 | Bleomycin | −5.378 |
| 78 | Bleomycin | −5.371 |
| 79 | NADH | −5.348 |
| 80 | Bleomycin | −5.332 |
| 81 | Theophylline | −5.308 |
| 82 | Entecavir | −5.3 |
| 83 | Bleomycin | −5.283 |
| 84 | Edotreotide gallium Ga-68 | −5.272 |
| 85 | Bleomycin | −5.244 |
| 86 | Digoxin | −5.244 |
| 87 | Bleomycin | −5.221 |
| 88 | Bleomycin | −5.215 |
| 89 | Bleomycin | −5.204 |
| 90 | Bleomycin | −5.185 |
| 91 | Bleomycin | −5.183 |
| 92 | Edotreotide gallium Ga-68 | −5.181 |
| 93 | Bleomycin | −5.175 |
| 94 | Leuprolide | −5.171 |
| 95 | Nelfinavir | −5.15 |
| 96 | Bleomycin | −5.136 |
| 97 | Edotreotide gallium Ga-68 | −5.132 |
| 98 | Bleomycin | −5.127 |
| 99 | Bleomycin | −5.12 |
| 100 | Bleomycin | −5.117 |

TABLE 3
Example Relaxin Antagonists and Docking Scores.

| Entry ID | GENERIC NAME | docking score |
|---|---|---|
| 1 | not used | not tested |
| 2 | Indium In-111 pentetreotide | −7.454 |
| 3 | Folic acid | −7.068 |
| 4 | NADH | −6.914 |
| 5 | Raltitrexed | −6.728 |
| 6 | Succinic acid | −6.37 |
| 7 | Capreomycin | −6.3 |
| 8 | Pentagastrin | −6.235 |
| 9 | Folic acid | −6.163 |
| 10 | Folic acid | −6.017 |
| 11 | Tenapanor | −5.975 |
| 12 | Chlorzoxazone | −5.719 |
| 13 | Flavone | −5.659 |
| 14 | Hyaluronic acid | −5.649 |
| 15 | Anagrelide | −5.567 |
| 16 | Raltitrexed | −5.552 |
| 17 | Mesalazine | −5.522 |
| 18 | Adenosine phosphate | −5.496 |
| 19 | Phenylbutyric acid | −5.384 |
| 20 | Argatroban | −5.376 |
| 21 | Phenylpropanolamine | −5.282 |
| 22 | Calcium acetate | −5.234 |
| 23 | Troglitazone | −5.212 |
| 24 | Isoetharine | −5.137 |
| 25 | Salicylamide | −5.129 |
| 26 | Indium In-111 pentetreotide | −5.111 |
| 27 | Biotin | −5.095 |
| 28 | Carboprost tromethamine | −5.08 |
| 29 | Octreotide | −4.977 |
| 30 | Iomeprol | −4.968 |
| 31 | Pralatrexate | −4.955 |
| 32 | Cefpiramide | −4.952 |
| 33 | Troglitazone | −4.935 |
| 34 | Lipoic acid | −4.928 |
| 35 | Troglitazone | −4.922 |
| 36 | Glutamic acid | −4.882 |
| 37 | Tryptophan | −4.858 |
| 38 | Cidofovir | −4.824 |
| 39 | Dicoumarol | −4.82 |
| 40 | Iomeprol | −4.81 |
| 41 | Ertapenem | −4.793 |
| 42 | Mupirocin | −4.777 |
| 43 | Phenylephrine | −4.762 |
| 44 | Norepinephrine | −4.75 |
| 45 | Troglitazone | −4.726 |
| 46 | Tranexamic acid | −4.719 |
| 47 | Minoxidil | −4.715 |
| 48 | Pyrithione | −4.698 |
| 49 | Isoetharine | −4.674 |
| 50 | Methysergide | −4.674 |
| 51 | Plicamycin | −4.661 |
| 52 | Pamidronic acid | −4.659 |
| 53 | Indecainide | −4.645 |
| 54 | Mangafodipir | −4.642 |
| 55 | Tiopronin | −4.64 |
| 56 | Nitisinone | −4.618 |
| 57 | Ertapenem | −4.589 |
| 58 | Zoledronic acid | −4.566 |
| 59 | Octreotide | −4.557 |
| 60 | Mefloquine | −4.534 |
| 61 | Raltegravir | −4.527 |
| 62 | Treprostinil | −4.514 |
| 63 | Cefotiam | −4.514 |
| 64 | Digoxin | −4.497 |
| 65 | Amphetamine | −4.495 |
| 66 | Unoprostone | −4.495 |
| 67 | Capreomycin | −4.489 |
| 68 | Phentermine | −4.487 |
| 69 | Tenapanor | −4.485 |
| 70 | Protriptyline | −4.483 |
| 71 | Protokylol | −4.447 |
| 72 | Milrinone | −4.429 |
| 73 | Ceftazidime | −4.423 |
| 74 | Indium In-111 pentetreotide | −4.409 |
| 75 | Lorazepam | −4.406 |
| 76 | Pamidronic acid | −4.382 |
| 77 | Pyrazinamide | −4.378 |
| 78 | Valrubicin | −4.378 |
| 79 | Methocarbamol | −4.364 |
| 80 | Pralatrexate | −4.355 |
| 81 | Chlorthalidone | −4.351 |
| 82 | Zoledronic acid | −4.349 |
| 83 | Calcium glucoheptonate | −4.329 |
| 84 | Glutathione | −4.318 |
| 85 | Tezacaftor | −4.312 |
| 86 | Sapropterin | −4.306 |
| 87 | Phenoxymethylpenicillin | −4.289 |
| 88 | Midodrine | −4.285 |
| 89 | Miglustat | −4.273 |
| 90 | Betazole | −4.271 |
| 91 | Penciclovir | −4.257 |
| 92 | Deferiprone | −4.214 |
| 93 | Nonoxynol−9 | −4.19 |
| 94 | Trihexyphenidyl | −4.181 |
| 95 | Bleomycin | −4.154 |
| 96 | Palonosetron | −4.138 |
| 97 | Tiopronin | −4.132 |
| 98 | Indium In-111 pentetreotide | −4.125 |
| 99 | Desmopressin | −4.116 |
| 100 | Midodrine | −4.113 |

Any of the compounds can be specifically claimed that show activity. The examples are not limiting of the technology.

### Example 2. Expression of Relaxin-2 Receptor in ACL Explants from Both Female and Male Patients

Investigation directive: Extent that RXFP2 receptors and apoptotic cells are differentially expressed in ACL tissue of male and female patients with ACL reconstruction.

Methods: Biopsy tissues of the ACL in the knee were collected from 20 patients with ACL reconstruction (ages ranging from 16 years to 61 years old, 10 males and 10 females). The normal ACLs were obtained from the rejected human knee osteochondral grafts from the Musculoskeletal Transplant Foundation (MTF) or the donors from MedCure (ages ranging from 24 years to 84 years old, 4 male and 4 females) were used as control samples. Immunohistochemistry was performed to detect the expression of RXFP2 and apoptosis. The number of RXFP2 positive and apoptotic positive cells were counted from six different fields for each patient. ACL cells were cultured in vitro, then cells were treated with relaxin-2 at different concentrations. After treatment, cells were lysed and analyzed for the expressions of MMP1 and MMP13 using quantitative real-time polymerase chain reaction. The protocol for patient enrollment was approved by the IRB committee at Rhode Island Hospital. GraphPad PRISM 8 software was used to perform all statistical analyses. Results were presented as mean±standard error of the mean (SEM) of different samples. Data were examined by one-way analysis of variance (ANOVA) with Tukey post-hoc multiple comparison. An unpaired t-test was used to compare any two groups. A value of p<0.05 was considered to be statically significant.

### Sample Collection

Discarded ACL tissues were collected following ACL reconstruction surgeries from 20 patients (10 males and 10 females) who had authorized the use of their tissues to be harvested for this study. The healthy ACL tissues from rejected donor's left and right knee osteochondral grafts obtained from the Musculoskeletal Transplant Foundation (MTF). The other normal ACLs were received from the donors of MedCure. The donors' age is ranging from 24 years to 84 years old (4 male and 4 females) and donors' ACLs were used as control samples. Harvested patient ACL tissues were placed in a normal saline. Part of every patient ACL or donors' ACLs were fixed in 4% of paraformaldehyde solution for immunohistochemical staining, and another part of patients' ACL tissues were used for cell culture. The protocol for patient enrollment was approved by the IRB committee at our institution.

### Cell Culture, Passaging and Harvesting

Patient ACL tissues were minced and digested with 1.0 mg/mL of type II collagenase at 37° C. at 5% CO₂ in 2 mL Dulbecco's modified Eagle medium F12 (DMEM/F12, Gibco) containing 1% antibiotic-antimycotic (Gibco) for 12 hours. ACL cells were then filtered through a 70-mm nylon mesh filter and collected by centrifugation. ACL cells were plated at a density of 1×10⁵ cells/cm² in DMEM/F12 media supplemented with 10% fetal bovine serum (FBS, Gibco) and 1% antibiotic-antimycotic in 20 cm petri dishes for passage one. The media was changed twice a week. Following one passage, ACL cells in the petri dishes were washed with Phosphate Buffered Saline (PBS). Trypsin was then applied for 5-8 minutes to detach cells from the culture dishes. The DMEM/F12 media with FBS was then added to the cells to stop the trypsin reaction. At passage 2, patient ACL cells were cultured in 10 cm petri dishes in DMEM/F12 completed medium, containing 10% FBS, 1% penicillin-streptomycin (10,000 U/ml), and 1% antibiotic-antimycotic at 37° C., 5% CO₂ in a humidified incubator. Dishes containing single-patient-derived ACL cells around 80% confluence were incubated with 1 ng/mL, 10 ng/mL, or 100 ng/mL of recombinant human relaxin-2 (R&D Systems) in serum-free DMEM/F12 containing 0.2% lactalbumin hydrolysate (Sigma) and 1% antibiotic-antimycotic. Cell morphology was observed under microscopy. Cultured cells stopped growing when they reached confluence (contact inhibition).

### Immunohistochemical Staining

The ACL tissues of patients and donors were immersed in 4% of paraformaldehyde and fixed for 3 days. The fixed ACL tissues were then embedded in paraffin, cut into 6 m histologic sections, and placed on glass slides. Next, the slides were deparaffinized with 3 changes of xylene, and rehydrated with 2 changes of 100% ethanol, 2 changes of 95% ethanol, and 1 change of 70% ethanol, before being washed with distilled water. Immunohistochemical Staining was then performed on the ACL tissues. First, antigen retrieval was achieved by steaming the slides in a 0.01M sodium citrate buffer (pH 6.0) at 99-100° C. for 20 minutes and cooling for 20 minutes at room temperature. The sections were then washed 3 times with phosphate buffered saline with Tween 20 (PBST) for 5 minutes each. Primary antibodies of RXFP2, MMP1, MMP3, and MMP13 were then applied to the sections and left overnight at 4° C. Negative controls were applied with PBST instead of the primary antibody. The following day, the slides were washed with PBST again and Primary Antibody Enhancer was applied for 10 minutes at room temperature from the UltraVision LP Detection System HRP DAB kit (Thermo Fisher Scientific, Waltham, MA, USA). Following another wash of PBST, HRP Polymer was applied for 15 minutes at room temperature out of direct light. After awash of PBST, a mixture of diaminobenzidine tetrahydrochloride (DAB) Quanto Chromogen and DAB Quanto Substrate was added to tissues on the slides, and allowed to incubate for 3-3.5 minutes. The slides were then washed in deionized water for 1 minute at a time and stained with hematoxylin for nucleus detection. Finally, the slides were dehydrated with 3 changes of 95% ethanol, 2 changes of 100% ethanol, and 3 changes of xylene for 1 minute each, before 1-3 drops of Cytoseal 60 mounting medium were added. Acover slip was placed over the stained tissue.

### Quantification of RXFP2 Positive Cells

All stained slides from 20 patients' ACL tissues were captured as RGB images using the Nikon ECLIPSE Ni-E microscope (Nikon Inc., Melville, NY). For the immunostaining process, the area of the tissue and number of RXFP2 positive cells from 5-6 different fields per stain were counted using ImageJ in a blinded manner. The presence of the target antigen RXFP2 was indicated by the formation of a brown precipitate. The nucleus was stained blue to purple by hematoxylin staining. The combination of blue/purple and brown colors resulted in dark brown. The brown and dark brown cells were counted as RXFP2 positive cells. The number of RXFP2 positive cells in a 1 mm² field were calculated and compared between the male and female patients.

### Apoptosis Detection

After deparaffinizing and rehydrating tissue sections, the ApopTag® Peroxidase In Situ Apoptosis Detection Kit (EMD Millipore, Temecula, CA) was used to detect apoptotic cells in situ by labeling and detecting DNA strand breaks using the TUNEL method. The deparaffinized tissue sections were first incubated with freshly diluted proteinase K for 15 min at room temperature. Following 2 washes of distilled water for 2 minutes each, the slides were quenched in 3.0% hydrogen peroxide in PBS for 5 min at room temperature with two rinses of PBS for 5 minutes each afterwards. Excess liquid was gently tapped off, and the section was carefully blotted. Equilibrium buffer was then applied on the slides at 75 μl/5 cm² for 10 seconds at room temperature. After excess liquid was gently taped off and carefully aspirated around the section, 55 μl/5 cm² of working strength TdT enzyme was applied to the sections in a humidified chamber at 37° C. for 1 hour. Negative controls were applied with PBS instead of TdT. Next, the specimens were agitated for 15 seconds and incubated with a working strength stop/wash buffer. Each specimen then underwent three, 1 minute washes with PBS. After carefully blotting around the sections, 65 μl/5 cm² of anti-digoxigenin conjugates were applied to the slides and incubated in a humidified chamber for 30 minutes at room temperature. After four, 2 minute rinses of PBS, 75 μl/5 cm² of peroxidase substrate was applied to cover the specimens for 3 minutes. Afterwards, the specimens were washed with three rinses of distilled water for 1 minute each before incubating in distilled water for 5 minutes. After counterstaining the slides in 0.5% methyl green solution for 10 minutes at room temperature, the sections were washed with three changes of distilled water and three changes of 100% N-butanol. The slides were then dehydrated with the same procedure mentioned previously and mounted with a coverslip.

### Quantification of Apoptosis Positive Cells

All stained slides from 20 patients' ACL tissues were captured as RGB images using the Nikon ECLIPSE Ni-E microscope (Nikon Inc., Melville, NY). For all the immunostaining, the area of the tissue and number of apoptosis positive cells from 6 different fields per stain were counted using ImageJ in a blinded manner. The presence of apoptosis was indicated by the formation of a brown precipitate. The nucleus was stained green by methyl green staining. The brown cells were counted as apoptosis positive cells. The number of apoptosis positive cells in a 1 mm² field were calculated and compared between the male and female patients.

Cyclic Adenosine Monophosphate (cAMP) Detection

The detection of cAMP in samples was conducted using Abcam's cAMP Complete in vitro competitive Enzyme-Linked Immunosorbent Assay (ELISA) kit. Patient samples were first thawed from −80° C. and added to a 10% protease inhibitor in RIPA buffer (Thermo Scientific) solution for cell lysis. Each sample was then pipetted up and down 10 times with a syringe every 10 minutes over the course of 30 minutes. Next, the samples were centrifuged at 14,000 RPM for 5 minutes. The supernatant was then extracted from the pellet and stored at −80° C.

Following cell lysis and supernatant extraction, the total protein concentration of the samples was determined using a BCA protein assay. Albumin standards were prepared with the RIPA buffer and protease inhibitor. Standards were prepared at concentrations of 2 mg/mL, 1 mg/mL, 0.5 mg/mL, 0.25 mg/mL, 0.125 mg/mL, 0.063 mg/mL, and 0 mg/mL. Patient samples were diluted to 1:1 and 1:5 concentrations of sample using RIPA buffer and protease inhibitor. Standards and samples were then loaded in a 96 well plate with BCA reagent and was read in a plate reader at 570 nm to determine overall protein concentrations in samples. The concentrations of cAMP were determined using Abcam's ELISA kit. According to the manufacturer's instructions, ACL cell lysates were diluted to 1:2 and 1:5 with 0.5% Triton in hydrochloric acid (HCl). Standards from the manufacturer kit were diluted to concentrations of 20 pmol/mL, 5 pmol/mL, 1.25 pmol/mL, 0.312 pmol/mL, and 0.078 pmol/mL. The samples and standards were then loaded in a 96 well plate with duplicates. In addition, wells for blanks (B₅), containing only substrate, total activity (TA), containing 5 μL conjugate and substrate, non-specific binding (NSB), containing standard diluent, assay buffer, conjugate, and substrate, and 0 pmol/mL standard (B₀) containing standard diluent, conjugate, antibody, and substrate. Neutralizing reagent was added to all wells, excluding TA and blank wells; cAMP Alkaline Phosphatase was added to all wells, excluding TA and B₅ wells; and Cyclic AMP Complete Antibody was added into all wells, excluding the B₅, TA, and NSB wells. The plate was then incubated at room temperature on a plate shaker for 2 hours at 500 RPM. The contents of the wells were then discarded and washed 3 times with a 1× wash buffer, with aspirating following the last wash to remove any remaining wash buffer. cAMP Complete Alkaline Phosphatase was next added to the TA wells only. Afterwards, pNpp substrate solution was added to each well, and the plate was incubated at room temperature for an hour without shaking. Stop solutions were added, and the plate was read at 405 nm with correction between 570 nm and 590 nm to determine cAMP concentrations. cAMP concentrations in pmol/mg were calculated by dividing the cAMP concentrations in pmol/mL by the total protein concentrations from the BCA protein assay in mg/mL.

### Quantitative Real-Time RT-PCR

RNA was isolated using TRIzol reagent and RNeasy Mini Kit from cultured ACL cells. RNA concentrations and purity were evaluated using a Nanodrop spectrophotometer (Thermo Scientific, Wilmington, DE, USA). A total of 1 μg of sample RNA was reverse transcribed using the Quantitect RT Kit (Qiagen, cDNA was synthesized from RNA using the iScript cDNA synthesis kit (Bio-Rad). RNA concentrations and purity were evaluated using a Nanodrop spectrophotometer (Thermo Scientific, Wilmington, DE, USA). Exactly 500 ng DNA was used as the template for real-time PCR analyses. Quantitative PCR reactions were performed using SYBR Green Fast Mix in a Bio-Rad real-time PCR machine. All Taqman primer and probe mixes were from Thermo Fisher Scientific (Waltham, MA, USA). Changes in the gene expressions of MMP1 and MMP13 were measured, which were normalized to GAPDH.

### Statistical Analysis

GraphPad PRISM 8 software was used to perform all statistical analyses. Results were presented as mean±standard error of the mean (SEM) of different samples. Data were examined by one-way analysis of variance (ANOVA) with Tukey post-hoc multiple comparison. An unpaired t-test was used to compare any two groups. A value of p<0.05 was considered to be statically significant.

### Results: Patient Demographics and Clinical Characteristics

ACL samples of 20 patients (10 males and 10 females), 4 normal males and 4 normal females were collected. Patient demographics and clinical characteristic are described in Table 1. The mean age of all patients was 30.6 years old, with patient ages ranging from 16 years to 61 years old. The mean age of 10 male patients was 31.4±4.15, and the mean age of 10 female patients was 29.8±4.29. The mean age of 4 normal male donors was 51±13.18, and the mean age of 4 normal female donors was 58.75±12.63, with donors' age ranging from 24 years to 84 years old (FIG. 1A). There was no discernible difference on age between male and female patients. The age of normal female donors was significantly higher than that of female patients.

### RXFP2 Expression in the ACL Tissues

Immunohistochemical staining of a relaxin-2 receptor RXFP2 was performed on ACL samples from all 20 male and female patient samples. Nuclei were indicated by blue/purple hematoxylin staining (FIG. 1B). This served as a negative control. The presence of the target antigen RFXP2 was indicated by the presence of a brown or dark brown precipitate (FIG. 1B). The dark brown precipitate resulted when the blue/purple stained nuclei were combined with the RXFP2 brown staining. We found that both male and female ACL samples of patients and normal donors expressed RXFP2. The number of RXFP2 positive cells was found to be markedly higher in ACL tissues from female patients as compared to that of male patients or normal female donors. There was no difference on the number of RXFP2 positive cells among normal male donors, normal female donors and male patients. The mean RXFP2 numbers of human ACLs were as follows: Normal male donor was 91.5±3.97, normal female donor was 110.8±8.38, male patient was 137.4±24.83, and female patient was 265.6±43.25 (FIG. 1C). These results suggest that the activity of relaxin-2 may be related to gender and may be more upregulated in female ACL tissues than that of males.

Data were examined using Pearson's correlation test. The correlation coefficient r was 0.4595 (r²=0.2112, p=0.1815) for males in FIG. 1D, r was 0.2483 (r²=0.06165, p=0.4891) for female patients in FIG. 1E, and r was 0.22352 (r²=0.05531, p=0.3182) for all patients in FIG. 1F. These results indicate that a weak relationship exists between RXFP2 expression and age in ACLs from these 20 patients, and in female patients. There was a significant correlation between RXFP2 expression and age in male patients.

### Apoptotic Expression in the ACL Tissues

Both male and female ACL samples of patients and normal donors expressed apoptosis. Apoptotic expression was found to be significantly higher in female ACL tissues compared to that of male patients or normal female donors. The number of apoptotic cells in male patient's ACLs was markedly higher than that of normal male donors. There was no difference on the number of apoptotic cells between normal male donors and normal female donors. The mean apoptotic cell number of normal male donor was 101±13.04, normal female donor was 114±15.74, 10 male patients was 253±41.93, and 10 female patients was 422.7±58.49 (FIG. 2A and FIG. 2B). These results suggest that increased apoptotic activity may be related to gender and may be more upregulated in female ACL tissues than that of males after ACL injury. Correlation coefficient r was 0.7665 (r²=0.5875, p=0.0097) for males in FIG. 2C, r was 0.8174 (r²=0.6681, p=0.0039) for females in FIG. 2D, r was 0.7196 (r²=0.5178, p<0.0001) for all patients in FIG. 2E. These results indicate that a strong positive correlation was found between apoptotic cells and age of male patients, between apoptotic cells and female patients, between apoptotic cells and all 20 patients.

### Correlations Between Rxfp2 Expression and Apoptotic Cell Number

Correlation coefficient r was 0.5590 (r²=0.3125, p=0.0930) for males in FIG. 3A, r was −0.1195 (r²=0.01427, p=0.7424) for females in FIG. 3B, r was 0.3102 (r²=0.09622, p=0.1832) for all patients in FIG. 3C. These data showed there is a strong linear relationship between RXFP2 expression and the number of apoptotic cells in male patients. There is a moderate positive relationship between RXFP2 expression and the number of apoptotic cells in all 20 patients. There is no relationship between RXFP2 expression and the number of apoptotic cells in female patients.

### Expression of Camp in the ACL Tissues

Relaxin-2 imparts extracellular catabolism in some types by binding to its receptor 1 and 2 as part of a cAMP-dependent pathway. The levels of cAMP were assessed using an enzyme-linked immunoassay for 17 patients. Patient 8, 10, and 17 (2 males and 1 female) were not analyzed due to insufficient cell growth. There was no significant difference found between cAMP concentrations in females and males. The mean cAMP concentration in the 8 male ACLs was 44.22±6.55 pmol/mg, and the mean cAMP concentration in the 9 female patients was 53.86±8.45 pmol/mg (FIG. 3D). There was also no significant correlation found between cAMP concentrations and age. The correlation coefficient r for cAMP and age was 0.2639 (r²=0.06967, p=0.5276) for male patients, r was −0.1789 (r²=0.03199, p=0.6452) for female patients, and r was −0.05598 (r²=0.003134, p=0.831) for patients overall (FIG. 3E and FIG. 3F). These data showed no correlation between cAMP concentration and 20 patients' age. These results indicate that there is no age-dependent change in cAMP concentration in ACL from male and female patients.

Correlations Between Apoptotic Cell Number and Time from Injury

Correlation coefficient r was −0.1357 (r²=0.01841, p=0.7086) for male patients, r was −0.0008151 (r²=6.643e-007, p=0.9982) for female patients, r was −0.08718 (r²=0.0076, p=0.7148) for all patients (FIG. 3G). These data showed there is no relationship between the number of apoptotic cells and time from injury in male patients, female patients and all 20 patients.

### Expressions of MMP1 and MMP13 in Patient ACL Tissues

The human ACL cells made progressive morphological changes during culture (FIG. 4A). Primary ACL cells were fibroblasts with a dark, round nucleus and short branches. Passaged ACL cells were fibroblasts with clustering, long branches, elongated and web-like shape. In female ACL cells, MMP1 expression increased in response to 100 ng/mL of relaxin-2, while MMP13 expression increased in response to 10 ng/mL of relaxin-2 as compared with untreated controls. In male ACL cells, relaxin-2 treatments didn't significant change the expressions of MMP1 and MMP3 as compared to control groups (FIG. 4B and FIG. 4C).

### Example 3. Effects of Relaxin-2 Antagonist on Cultured Anterior Cruciate Ligament (ACL) from Patients with ACL Reconstruction

Measurements of expressions of MMP13, collagen 1A, collagen 3A, MMP3, and MMP1 in patient ACL tissues were conducted (FIG. 5). Biopsy tissues of the ACL from 20 patients (10 male and 10 female) who underwent surgeries for ACL reconstruction were harvested. The patients' ages ranged from 16 to 61 years, with equal numbers of males and females. The protocol for patient enrollment was approved by the TRB committee at our institution. Each ACL tissue sample was minced and digested and then the cells were cultured and passaged before treated with different concentrations and combinations of 17β-estradiol (1 μM), relaxin-2 (100 ng/mL), relaxin-2 antagonist (Folic Acid, 10 μg/ml), and relaxin-2 receptor antagonist (NADH, 100 μg/ml).

After treatment, cells were lysed and analyzed for the expressions of type I collagen, type III collagen, MMP1, MMP3, and MMP13 using quantitative real-time polymerase chain reaction (RT-PCR). FIG. 5 provides expressions of MMP13, collagen 1A, collagen 3A, MMP3, and MMP1 in patient ACL tissues. (FIG. 5A) MMP13 expression; (FIG. 5B) collagen 1A expression; (FIG. 5C) collagen 3A expression; (FIG. 5D) MMP3 expression; (FIG. 5E) MMP1 expression. In FIG. 5, the protocol for patient enrollment was approved by the IRB committee at Rhode Island Hospital. GraphPad PRISM 8 software was used to perform all statistical analyses. Results were presented as mean+standard error of the mean (SEM) of different samples, and considered significant when p<0.05. A t-test was used to compare any two groups, *p<0.05, **p<0.01, ***p<0.001 compared to their individual control group; ##p<0.01, compared to their individual relaxin-2 group.

### Example 4. Animal Experiments (Ongoing and Future Work)

1. A Pilot Study to Determine the Optimal Dose is Undertaken with 108 Rats.

Female Sprague-Dawley rats (10-12 weeks old, weighing 250-300 g) are randomly divided in 9 groups, 12 rats/group:
(1) Control 1: an age-matched completely untreated group, tested at 10-12 weeks of age, to provide an indication of normal mechanical properties at 10-12 weeks;
(2) Control 2: an age-matched group treated with relaxin-2 treatment IV for 10 days, to demonstrate the effect of relaxin-2 treatment; and
(3) Control 3: an age-matched completely untreated group, tested at 10-12 weeks of age+20 days, to provide an indication of normal mechanical properties at ˜13-15 weeks.

Two sham groups: rats do not receive relaxin-2, but receive the inhibitors with the max dose of (4) relaxin-2 antagonist folic acid at 4 mg/kg or (5) relaxin-2 receptor antagonist NADH at 100 mg/kg, Rats receive relaxin-2 treatment IV×10 days.

Then the rats receive the antagonists: (6) Relaxin-2 antagonist (folic acid, 2 mg/kg), (7) Relaxin-2 antagonist (folic acid, 4 mg/kg), (8) Relaxin-2 receptor antagonist (NADH, 15 mg/kg), (9) Relaxin-2 receptor antagonist (NADH, 100 mg/kg). At Day 11-Day 31 rats receive one indicated treatments orogastrically gavaged daily from above.

After 20 days, the rats are euthanized for biomechanical testing. The dosage of drug that can achieve higher biomechanical properties (ACL strength, maximum loading before failure and stiffness) will be considered as the optimal concentration.

### 2. Follow-Up Study

According to power analysis, 12 rats will be used for each group. After completion of this pilot to determine optimal dose, total 48 rats will be used in this study. These 48 female Sprague-Dawley rats will be randomly divided into 4 groups (12 rats/group):

Group 1: Control group (volume matched saline) for 30 days;

Groups 2, 3 and 4 will receive relaxin-2 treatment IV×10 days. At Day 11-Day 31 rats will receive one of the following treatments: Group 2—Saline group; Group 3—Relaxin-2 antagonist (folic acid 2 mg/kg or 4 mg/kg, as determined by the pilot) group; Group 4—Relaxin-2 receptor antagonist (NADH 15 mg/kg or 100 mg/kg, as determined by the pilot) group.

Saline, Folic Acid, or NADH will be orogastrically gavaged 5 days per week×4 weeks. After which, the rats will be euthanized for post-mortem analysis including biomechanical testing, gene expression analysis, and histology and immunohistochemical staining. The experiments are simultaneously repeated using a dietary change, an encapsulant/food, and/or a precursor/prodrug of Folic Acid or NADH. The prodrug can be converted (to active) by an enzymatic activity of the host subject, a release from a carrier, a digestion such as by an amylase, or can be converted by a hydrolysis, wherein a hydroxyl in the parent compound is presented as an ester or a carbonate or carboxylic acid present in the parent compound is presented as an ester. Particles (such as silica) or even a food may be used as carriers/nanocarriers of the therapeutic agent(s) for prodrug/precursor experiments. A precursor or prodrug can be metabolized to the active parent compound (therapeutic agent) in vivo (e.g., the ester is hydrolyzed to the corresponding hydroxyl, or carboxylic acid). Formulation of a precursor/prodrug is accomplished by mixing Folic Acid or NADH with a carrier/nanocarrier, by mixing with an acid and/or an alcohol (then testing hydrolysis at various pH), or by attaching an enzymatically cleavable functional group. The design of prodrugs requires consideration of the following: (1) can the therapeutic agent be modified, (2) what effect will the modification have on the ADMET (absorption, distribution, metabolism, excretion, and toxicity), and (3) can the parent therapeutic agent be regenerated efficiently without producing toxic by-products. Chemical modification of Folic Acid or NADH requires a suitable functional group that allows reaction to form the targeted promoiety that bestows the desired ability for hydrolysis back to the therapeutic agent. Example groups include hydroxyls, amines, carboxylic acids, esters, thiols, and carbonyls. The purpose of the targeted promoiety generally dictates its targeted functions in formulation. For example, increased membrane permeability of a hydrophilic drug can be accomplished through lipidation (decreasing hydrophilicity), typically via ester bond formation. In doing so, the prodrug offers improved bioavailability through enhanced absorption from the gastrointestinal tract into systemic circulation or via topical application. Other routes to improve ADMET properties include the addition of ionizable groups to increase solubility (increasing hydrophilicity). The masking of metabolically labile groups is also investigated to prevent their premature breakdown. Future work contemplates the conjugation of peptidic epitopes for the active targeting of specific tissue/cell surface receptors and the site-sensitive activation of the precursor/prodrug for selective release.

### Postmortem Analysis: ACL Isolation and Load-to-Failure Testing

A cohort of the rats will be used for biomechanical testing. After rats are euthanized by CO₂ overdose, ACL isolation and biomechanical tests are conducted according to the methods described in a previous publication. Both hindlimbs are removed, and both femur-ACL-tibia complexes are isolated. All other tissues, with particular attention to non-ACL tissues also traversing the knee joint, are removed so that all that remained is the femur-ACL-tibia complex.

### RNA Isolation and Quantitative RT-PCR

A second cohort of the rats is used to evaluate ACL expression of collagen, MIP and relaxin receptor. After euthanasia, both ACLs from each rat are harvested and evaluated for the expression of relaxin receptor, MMP, and type I collagen and type III collagen.

### Histology and Immunohistochemical Staining

After euthanasia, the knees of the rats per treatment group are fixed in 10% formalin and embedded in paraffin, cut into 6 m histologic sections, and placed on glass slides. The slides are stained with hematoxylin and eosin. Immunohistochemical staining of relaxin receptor, MMPs, collagens and apoptosis are also performed on the harvested tissues.

### Example 5. Expression of a Relaxin-2 Receptor RXFP2 in the Shoulder Capsule and Labrum of Patients with Shoulder Instability

### Materials and Methods: Study Design and Ethics Statement

Twenty patients undergoing surgical treatment for shoulder instability consented to the biopsy of their labrum and capsule from January 2019 to December 2019. Subjects including 15 males and 5 females with ages between 18 and 40 years old (mean age is 24.6 years). This study was approved by the IRB committee at our institution.

### Patient Specimen Collection

During the surgery, a 2 mm×2 mm piece of shoulder labrum and capsule was collected from 20 patients with shoulder instability. The specimens were harvested from the anterior-inferior labrum at the 5 or 7 o'clock position. The capsular biopsy was similarly performed 10 mm anterior to the labral biopsy site. Once harvested, the labra and capsules were stored in 4% of paraformaldehyde solution.

### Immunohistochemical Staining of RXFP2

Immunostaining was performed on paraffin-embedded labrum and capsule tissues. In brief, the paraffin blocks were cut into 5 m histologic sections, deparaffinized with xylene and rehydrated in an ethanol series. Antigen retrieval was achieved by steaming the slides in 0.01M sodium citrate buffer (pH 6.0) at 99-100° C. for 20 minutes and cooling for 20 minutes at room temperature. The staining was performed with an UltraVision LP Detection System HRP DAB kit according to manufacturer's recommended protocol (Thermo Fisher Scientific, Waltham, MA, USA). Endogenous peroxidases were quenched by incubating the samples in UltraVision Hydrogen Peroxide Block for 10 minutes. The sections were washed 4 times with phosphate buffered saline with Tween 20 (PBST) for 5 minutes each time. An UltraVision Protein Block was applied for 5 minutes to block nonspecific background staining. The sections were then washed with PBST, then incubated with a primary RXFP2 antibody (LifeSpan BioSciences, Inc, Seattle, WA, USA) overnight at 4° C. After rinsing with PBST followed by applying Primary Antibody Enhancer and incubate for 10 minutes at room temperature. After washing with PBST, HRP Polymer was applied in dark for 15 minutes at room temperature. The slides were washed with PBST, then mixture of diaminobenzidine tetrahydrochloride (DAB) Quanto Chromogen and DAB Quanto Substrate was added to tissues on the slides to incubate for 3 minutes. After washing in deionized water for 1 minutes each time, the sections were stained with hematoxylin for nucleus. After dehydrating with increasing concentrations of ethanol and clearing in xylenes, 1-3 drops of Cytoseal 60 mounting medium were added and a cover slip was placed over the stained tissue. The brown precipitate indicates the presence of the target antigen RXFP2. The nucleus is stained purple by hematoxylin staining. Combination of purple and brown colors resulted in dark brown. The brown and dark brown cells were counted as RXFP2 positive cells.

### Quantification of RXFP2 Positive Cells

All stained slides from 20 patients' capsules and labra were scanned by Pannoramic MIDI II digital slide scanner (3DHISTECH Ltd., Budapest, Hungary) and the images were viewed and captured in CaseViewer 2.3 provided by 3DHISTECH Ltd., Budapest, Hungary. For all the immunostaining, the area of the tissue and number of RXFP2 positive cells from 5 to 7 different fields per stain were counted using the QuPath software (Version 0.2.0 for Windows 10) in a blinded manner. A QuPath software is created by the Centre for Cancer Research & Cell Biology at Queen's University Belfast, Northern Ireland, and developed at the University of Edinburgh, United Kingdom. The number of RXFP2 positive cells in 1 mm² field were calculated and compared between the male and female patients.

### Statistical Analysis

All statistical analyses were performed using GraphPad PRISM 8 software (GraphPad Software, Inc., San Diego, CA, USA). Results were presented as mean±standard deviation (SD) of different samples, and considered significant when p<0.05. Correlation analysis was done in two-tailed Pearson's correlation. Data were examined by one-way ANOVA with Bonferroni post-hoc multiple comparison to measure the effects of one factor. A t-test was used to compare any two groups.

RXFP2 was Expressed in Shoulder Capsules and Labra from Both Male and Female Subjects

Immunohistochemical staining of a relaxin-2 receptor RXFP2 were performed in shoulder capsule and labrum from all 20 male and female patients with shoulder instability. FIG. 6A (left panel) showed a negative control that was not incubated with a primary RXFP2 antibody during staining in human labrum. It has been reported that RXFP2 were present in shoulder joint capsule of young male Wistar rats.⁷⁴ Thus, we used shoulder labrum of male Wistar rat to stain RXFP2 as a positive control (right panel in FIG. 6A). As shown in FIG. 6B, RXFP2 was expressed in both shoulder capsules and labra from patients with shoulder instability. The number of RXFP2 positive cells in 1 mm2 field was 638±191 in male capsule, 230±37 in female capsule, 417±102 in male labrum, and 179±42 in female labrum. Statistical analysis showed that a significantly higher number of RXFP2 positive cells in 1 mm2 field was observed in capsules and labra in male than those in female from patients with shoulder instability (FIG. 6C). These results suggest that RXFP2 is expressed in both shoulder capsules and labra, and this is higher in male patients with shoulder instability compared to female subjects. FIG. 6 provides data supporting expression of RXFP2 on shoulder capsules and labra from 20 patients with shoulder instability.

Immunohistochemical staining of a relaxin-2 receptor RXFP2 was performed in patient shoulder specimens. The brown precipitate indicates the presence of the target antigen RXFP2. The nucleus is stained purple by hematoxylin staining. Combination of purple and brown colors resulted in dark brown. The brown and dark brown cells were counted as RXFP2 positive cells. (FIG. 6A) A representative image of a negative control and a positive control of immunohistochemistry. Negative control in human labrum tissues was not incubated with a RXFP2 antibody. RXFP2 immunohistochemistry in shoulder labrum of male Wistar rat was used as a positive control. (FIG. 6B) Representative immunohistochemistry images of RXFP2 positive cells in capsule and labrum tissues of patients with shoulder instability. Grayarrows indicate RXFP2 positive cells (brown to dark brown precipitate). (FIG. 6C) Quantification of the number of RXFP2 positive cells in 1 mm2 field from male patients (n=15) and female patients (n=5). Results were presented as mean±SD of different samples, as the dots are absolute number of RXFP2 positive cells. **P<0.01 vs male.

RXFP2 was Expressed in Blood Vessels and Nucleus of Shoulder Specimens from Male and Female Patients.

In FIG. 7A (upper panel), RXFP2 was widely expressed in blood vessels from the capsules of male and female patients with shoulder instability. In addition, RXFP2 was present in nucleus of capsules and labra of patients with shoulder instability (FIG. 7B). Nuclear expression of RXFP2 was shown as a dark brown staining after combination of purple (nuclear) and brown (RXFP2 positive) colors. These results demonstrate that RXFP2 can be expressed in vessels and nucleus of patients with shoulder instability. FIG. 7 supports expression of RXFP2 in vessels and nucleus in shoulder specimens from patients with shoulder instability. Immunohistochemical staining of a relaxin-2 receptor RXFP2 were performed in patient shoulder specimens. The brown precipitate indicates the presence of the target antigen RXFP2. The nucleus is stained purple by hematoxylin staining. Combination of purple and brown colors resulted in dark brown. The brown and dark brown cells were counted as RXFP2 positive cells. Representative immunohistochemistry images of RXFP2 located in vessels (FIG. 7A) and nucleus (FIG. 7B) of capsules and labra in patients with shoulder instability. Grayarrows indicate RXFP2 positive immunostaining (brown to dark brown precipitate). Black arrows indicate RXFP2 negative immunostaining (purple color).

RXFP2 Expression Did not Correlate with Patients' Age.

The mean age of 15 male patients was 25.0±1.9, and mean age of 5 female patients was 23.2±2.4. There was no statistic difference in age between male and female patients with shoulder instability (FIG. 8A). The number of RXFP2-positive cells varied on capsules (FIGS. 8B-D) and labra (FIGS. 8E-G) within these 20 patients. We then determined whether there is a correlation between RXFP2 expression and ages in patients with shoulder instability. As shown in FIG. 8B, correlation coefficient (r) between number of RXFP2 positive cells in capsules and ages was −0.1658 (p=0.5247). The r values between number of RXFP2 positive cells in capsules and ages in male and female patients were −0.3090 (p=0.3285) and 0.2826 (p=0.6450), respectively (FIG. 8C and FIG. 8D). The r value between number of RXFP2 positive cells in labra and ages was −0.3364 (p=0.1470) (FIG. 8E). The r values between number of RXFP2 positive cells in labra and ages in male and female patients were −0.4331 (p=0.1068) and −0.02334 (p=0.9703), respectively (FIG. 8F and FIG. 8G). These results indicate that there was no statistical significance in correlation between RXFP2 expression in capsules and labra and patients' age, regardless of sex. FIG. 8 supports there was no correlation between RXFP2 expression and ages in 20 patients with shoulder instability. Immunohistochemical staining of a relaxin-2 receptor RXFP2 were performed in shoulder capsules and labra from 20 patients with shoulder instability. The number of RXFP2 positive cells in 1 mm² field were counted and the correlation between age and RXFP2 number in the male and female patients were calculated. (FIG. 8A) The age of male (n=15) and female (n=5) patients with shoulder instability was present. The correlation between age and RXFP2 number in 1 mm² field on capsules (FIG. 8B) of patients including twelve male (FIG. 8C) and five female (FIG. 8D) patients. The correlation between age and RXFP2 number in 1 mm² field on labra (FIG. 8E) of patients, including fifteen male (FIG. 8F) and five female (FIG. 8G) patients. The dots are absolute number of RXFP2 positive cells for each group.

### Example 6. Sex Differences of Vitamin D's Effects in Cultured Cells from Patients With Anterior Cruciate Ligament Reconstruction

### Materials and Methods

Biopsy tissues of the ACL in the knee were collected from 20 patients with ACL reconstruction (ages ranging from 16 years to 61 years old, 10 males and 10 females). The ACL cells were cultured in vitro and treated with vitamin D at different concentrations for 3 days. Cells were then lysed and analyzed for expressions of type I collagen and MMP13 genes using quantitative real-time polymerase chain reaction. The protocol for patient enrollment was approved by the IRB committee at Rhode Island Hospital. GraphPad PRISM 8 software was used to perform all statistical analyses. Results were presented as mean±standard error of the mean (SEM) of different samples and considered significant when p<0.05. A t-test was used to compare any two groups. See Appendix II for data and further discussion.

### Initial Results

Cells made progressive morphological changes during culture, and stopped growing when they reached confluence due to contact inhibition. Passaged ACL cells before treatment are fibroblasts, elongated, web-like and clustered for both male and female patients. In female cells, Type I collagen expression was increased after vitamin D treatments (1, 10 and 100 nmol/L) in female cells, whereas these effects were not observed in male cells. MMP13 gene expression was increased after vitamin D treatment at 1 nmol/L in both female and male cells. Interestingly, MMP13 gene expression was reduced after vitamin D treatments at 10 nmol/L in female cells, and at 100 nmol/L in male cells.

### Discussion

Female cells are more susceptible to vitamin D treatment in regulations of type I collagen or MMP13 gene expressions. Vitamin D binds to intracellular receptors, which function as transcription factors to regulate gene expression. Further study is warranted to evaluate levels of vitamin D and its receptors in both female and male cells treated with vitamin D. This study provides mechanisms underlying sex differences in developing ACL ruptures and provides the evidence to develop potential drugs candidates to treat or prevent ACL injury.

As used throughout, it is understood that any female cell or female subject is wherein a female subject (or a cell derived therefrom such) is optionally a non-male subject or a subject incapable of producing one or more gametes capable of fertilizing an egg of another non-male or another female. It should be understood that while the data disclosed herein was surprising in distinct differences between females and males, the invention can be utilized for treatments on any subject. It should be understood that the vitamin D effects can be used in combination with any of the other surprising discoveries disclosed herein.

### REFERENCES

1. Abram SGF, Judge A, Khan T, Beard D J, Price A J. Rates of knee arthroplasty in anterior cruciate ligament reconstructed patients: a longitudinal cohort study of 111,212 procedures over 20 years. Acta Orthop. 2019; 90(6):568-574.
2. Acevedo R J, Rivera-Vega A, Miranda G, Micheo W. Anterior cruciate ligament injury: identification of risk factors and prevention strategies. Curr Sports Med Rep. 2014; 13(3):186-191.
3. Agoulnik A I. Relaxin and related peptides in male reproduction. Adv Exp Med Biol. 2007; 612:49-64.
4. Aliashrafi S, Ebrahimi-Mameghani M, Jafarabadi M A, et al. Effect of high-dose vitamin D supplementation in combination with weight loss diet on glucose homeostasis, insulin resistance, and matrix metalloproteinases in obese subjects with vitamin D deficiency: a double-blind, placebo-controlled, randomized clinical trial. Applied Physiology, Nutrition, and Metabolism. 2020; 45:1092+.
5. Angeline M E, Ma R, Pascual-Garrido C, et al. Effect of diet-induced vitamin D deficiency on rotator cuff healing in a rat model. Am J Sports Med. 2014; 42(1):27-34.
6. Bani D, Nistri S, Cinci L, et al. A novel, simple bioactivity assay for relaxin based on inhibition of platelet aggregation. Regul Pept. 2007; 144(1-3):10-16.
7. Barker T, Henriksen V T, Martins T B, et al. Higher serum 25-hydroxyvitamin D concentrations associate with a faster recovery of skeletal muscle strength after muscular injury. Nutrients. 2013; 5(4):1253-1275.
8. Barker T, Henriksen V T, Rogers V E, et al. Vitamin D deficiency associates with γ-tocopherol and quadriceps weakness but not inflammatory cytokines in subjects with knee osteoarthritis. Redox Biol. 2014; 2:466-474.
9. Barker T, Martins T B, Hill H R, et al. Low Vitamin D Impairs Strength Recovery After Anterior Cruciate Ligament Surgery. Journal of Evidence-Based Complementary & Alternative Medicine. 2011; 16(3):201-209.
10. Bathgate R A, Halls M L, van der Westhuizen E T, et al. Relaxin family peptides and their receptors. Physiol Rev. 2013; 93(1):405-480.
11. Bathgate R A, Samuel C S, Burazin T C, Gundlach A L, Tregear G W. Relaxin: new peptides, receptors and novel actions. Trends Endocrinol Metab. 2003; 14(5):207-213.
12. Baugher W H, Warren R F, Marshall J L, Joseph A. Quadriceps atrophy in the anterior cruciate insufficient knee. Am J Sports Med. 1984; 12(3):192-195.
13. Bhat M, Kalam R, Qadri S S, Madabushi S, Ismail A. Vitamin D deficiency-induced muscle wasting occurs through the ubiquitin proteasome pathway and is partially corrected by calcium in male rats. Endocrinology. 2013; 154(11):4018-4029.
14. Bisson L J, Gurske-DePerio J. Axial and sagittal knee geometry as a risk factor for noncontact anterior cruciate ligament tear: a case-control study. Arthroscopy. 2010; 26(7):901-906.
15. Blessing W A, Okajima S M, Cubria M B, et al. Intraarticular injection of relaxin-2 alleviates shoulder arthrofibrosis. Proc Natl Acad Sci USA. 2019; 116(25):12183-12192.
16. Bogunovic L, Kim A D, Beamer B S, Nguyen J, Lane J M. Hypovitaminosis D in patients scheduled to undergo orthopaedic surgery: a single-center analysis. J Bone Joint Surg Am. 2010; 92(13):2300-2304.
17. Bonaventure J, de La Tour B, Tsagris L, et al. Effect of relaxin on the phenotype of collagens synthesized by cultured rabbit chondrocytes. Biochim Biophys Acta. 1988; 972(2):209-220.
18. Buckthorpe M, Danelon F, La Rosa G, et al. Recommendations for Hamstring Function Recovery After ACL Reconstruction. Sports Med. 2021; 51(4):607-624.
19. Callander G E, Bathgate R A. Relaxin family peptide systems and the central nervous system. Cell Mol Life Sci. 2010; 67(14):2327-2341.
20. Cancienne J M, Brockmeier S F, Kew M E, Werner B C. Perioperative Serum 25-Hydroxyvitamin D Levels Affect Revision Surgery Rates After Arthroscopic Rotator Cuff Repair. Arthroscopy. 2019; 35(3):763-769.
21. Cevallos N, Soriano KKJ, Lansdown D A, et al. Contemporary Practice Patterns for the Treatment of Anterior Cruciate Ligament Tears in the United States. Orthop J Sports Med. 2021; 9(9):23259671211040891.
22. Clifton K B, Rodner C, Wolf J M. Detection of relaxin receptor in the dorsoradial ligament, synovium, and articular cartilage of the trapeziometacarpal joint. J Orthop Res. 2014; 32(8):1061-1067.
23. Close G L, Russell J, Cobley J N, et al. Assessment of vitamin D concentration in non-supplemented professional athletes and healthy adults during the winter months in the U K: implications for skeletal muscle function. J Sports Sci. 2013; 31(4):344-353.
24. Colwell C W, Jr., D'Lima D D, Hoenecke H R, et al. In vivo changes after mechanical injury. Clin Orthop RelatRes. 2001(391 Suppl):S116-123.
25. Conrad K P, Baker V L. Corpus luteal contribution to maternal pregnancy physiology and outcomes in assisted reproductive technologies. Am J Physiol Regul Integr Comp Physiol. 2013; 304(2):R69-72.
26. D'Elia H F, Mattsson L A, Ohlsson C, Nordborg E, Carlsten H. Hormone replacement therapy in rheumatoid arthritis is associated with lower serum levels of soluble IL-6 receptor and higher insulin-like growth factor 1. Arthritis Res Ther. 2003; 5(4):R202-209.
27. de la Puente Yague M, Collado Yurrita L, Ciudad Cabanas M J, Cuadrado Cenzual M A. Role of Vitamin D in Athletes and Their Performance: Current Concepts and New Trends. Nutrients. 2020; 12(2).
28. Dehghan F, Haerian B S, Muniandy S, et al. The effect of relaxin on the musculoskeletal system. Scand J Med Sci Sports. 2014; 24(4):e220-229.
29. Dehghan F, Muniandy S, Yusof A, Salleh N. Testosterone reduces knee passive range of motion and expression of relaxin receptor isoforms via 5alpha-dihydrotestosterone and androgen receptor binding. Int J Mol Sci. 2014; 15(3):4619-4634.
30. Domnick C, Raschke M J, Herbort M. Biomechanics of the anterior cruciate ligament: Physiology, rupture and reconstruction techniques. World J Orthop. 2016; 7(2):82-93.
31. Dougherty K A, Dilisio M F, Agrawal D K. Vitamin D and the immunomodulation of rotator cuff injury. J Inflamm Res. 2016; 9:123-131.
32. Dragoo J L, Castillo T N, Braun H J, et al. Prospective correlation between serum relaxin concentration and anterior cruciate ligament tears among elite collegiate female athletes. Am J Sports Med. 2011; 39(10):2175-2180.
33. Dragoo J L, Lee R S, Benhaim P, Finerman G A, Hame S L. Relaxin receptors in the human female anterior cruciate ligament. Am J Sports Med. 2003; 31(4):577-584.
34. Dragoo J L, Padrez K, Workman R, Lindsey D P. The effect of relaxin on the female anterior cruciate ligament: Analysis of mechanical properties in an animal model. Knee. 2009; 16(1):69-72.
35. Duthon V B, Barea C, Abrassart S, et al. Anatomy of the anterior cruciate ligament. Knee Surg Sports Traumatol Arthrosc. 2006; 14(3):204-213.
36. Eljabu W, Klinger H M, von Knoch M. The natural course of shoulder instability and treatment trends: a systematic review. J Orthop Traumatol. 2017; 18(1):1-8.
37. Elmore S. Apoptosis: a review of programmed cell death. Toxicol Pathol. 2007; 35(4):495-516.
38. Facciolli A, Ferlin A, Gianesello L, Pepe A, Foresta C. Role of relaxin in human osteoclastogenesis. Ann NY Acad Sci. 2009; 1160:221-225.
39. Faryniarz D A, Bhargava M, Lajam C, Attia E T, Hannafin J A. Quantitation of estrogen receptors and relaxin binding in human anterior cruciate ligament fibroblasts. In Vitro Cell Dev Biol Anim. 2006; 42(7):176-181.
40. Feng S, Bogatcheva N V, Truong A, et al. Developmental expression and gene regulation of insulin-like 3 receptor RXFP2 in mouse male reproductive organs. Biol Reprod. 2007; 77(4):671-680.
41. Feng S, Ferlin A, Truong A, et al. INSL3/RXFP2 signaling in testicular descent. Ann NY Acad Sci. 2009; 1160:197-204.
42. Ferrari R, Prosser C. Testing Vitamin D Levels and Choosing Wisely. JAMA Internal Medicine. 2016; 176(7):1019-1020.
43. Figueiredo K A, Mui A L, Nelson C C, Cox M E. Relaxin stimulates leukocyte adhesion and migration through a relaxin receptor LGR7-dependent mechanism. J Biol Chem. 2006; 281(6):3030-3039.
44. Fleming B C, Hulstyn M J, Oksendahl H L, Fadale P D. Ligament Injury, Reconstruction and Osteoarthritis. Curr Opin Orthop. 2005; 16(5):354-362.
45. Garcia L A, King K K, Ferrini M G, Norris K C, Artaza J N. 1,25(OH)2vitamin D3 stimulates myogenic differentiation by inhibiting cell proliferation and modulating the expression of promyogenic growth factors and myostatin in C2C12 skeletal muscle cells. Endocrinology. 2011; 152(8):2976-2986.
46. Garibay-Tupas J L, Okazaki K J, Tashima L S, Yamamoto S, Bryant-Greenwood G D. Regulation of the human relaxin genes H1 and H2 by steroid hormones. Mol Cell Endocrinol. 2004; 219(1-2):115-125.
47. Goh W, Yamamoto S Y, Thompson K S, Bryant-Greenwood G D. Relaxin, its receptor (RXFP1), and insulin-like peptide 4 expression through gestation and in placenta accreta. Reprod Sci. 2013; 20(8):968-980.
48. Goldsmith L T, Weiss G. Relaxin in human pregnancy. Ann NY Acad Sci. 2009; 1160:130-135.
49. Gordon P L, Sakkas G K, Doyle J W, Shubert T, Johansen K L. Relationship between vitamin D and muscle size and strength in patients on hemodialysis. J Ren Nutr. 2007; 17(6):397-407.
50. Granstrom L M, Ekman G E, Malmstrom A, Ulmsten U, Woessner J F, Jr. Serum collagenase levels in relation to the state of the human cervix during pregnancy and labor. Am J Obstet Gynecol. 1992; 167(5):1284-1288.
51. Grassi A, Macchiarola L, Lucidi G A, et al. More Than a 2-Fold Risk of Contralateral Anterior Cruciate Ligament Injuries Compared With Ipsilateral Graft Failure 10 Years After Primary Reconstruction. Am J Sports Med. 2020; 48(2):310-317.
52. Griffin L Y, Agel J, Albohm M J, et al. Noncontact anterior cruciate ligament injuries: risk factors and prevention strategies. J Am Acad Orthop Surg. 2000; 8(3):141-150.
53. Griffin L Y, Albohm M J, Arendt E A, et al. Understanding and preventing noncontact anterior cruciate ligament injuries: a review of the Hunt Valley II meeting, January 2005. Am J Sports Med. 2006; 34(9):1512-1532.
54. Gupta R, Singhal A, Kapoor A, et al. Vitamin D deficiency in athletes and its impact on outcome of Anterior Cruciate Ligament surgery. Eur J Orthop Surg Traumatol. 2021; 31(6):1193-1197.
55. Guttridge D C. Signaling pathways weigh in on decisions to make or break skeletal muscle. Curr Opin Clin Nutr Metab Care. 2004; 7(4):443-450.
56. Halls M L, van der Westhuizen E T, Bathgate R A, Summers R J. Relaxin family peptide receptors—former orphans reunite with their parent ligands to activate multiple signalling pathways. Br J Pharmacol. 2007; 150(6):677-691.
57. Harada G K, Arshi A, Fretes N, et al. Preoperative Vitamin D Deficiency Is Associated With Higher Postoperative Complications in Arthroscopic Rotator Cuff Repair. J Am Acad Orthop Surg Glob Res Rev. 2019; 3(7):e075.
58. Hasegawa A, Otsuki S, Pauli C, et al. Anterior cruciate ligament changes in the human knee joint in aging and osteoarthritis. Arthritis Rheum. 2012; 64(3):696-704.
59. Hayami T, Kapila Y L, Kapila S. MMP-1 (collagenase-1) and MMP-13 (collagenase-3) differentially regulate markers of osteoblastic differentiation in osteogenic cells. Matrix Biol. 2008; 27(8):682-692.
60. Hellio Le Graverand M P, Reno C, Hart D A. Influence of pregnancy on gene expression in rabbit articular cartilage. Osteoarthritis Cartilage. 1998; 6(5):341-350.
61. Hewett T E, Zazulak B T, Myer G D. Effects of the menstrual cycle on anterior cruciate ligament injury risk: a systematic review. Am J Sports Med. 2007; 35(4):659-668.
62. Hinek A, Poole A R. The influence of vitamin D metabolites on the calcification of cartilage matrix and the C-propeptide of type II collagen (chondrocalcin). J Bone Miner Res. 1988; 3(4):421-429.
63. Ho T Y, Santora K, Chen J C, Frankshun A L, Bagnell C A. Effects of relaxin and estrogens on bone remodeling markers, receptor activator of NF-kB ligand (RANKL) and osteoprotegerin (OPG), in rat adjuvant-induced arthritis. Bone. 2011; 48(6):1346-1353.
64. Holick M F. The vitamin D deficiency pandemic: Approaches for diagnosis, treatment and prevention. Rev EndocrMetab Disord. 2017; 18(2):153-165.
65. Holick M F, Chen T C. Vitamin D deficiency: a worldwide problem with health consequences. Am J Clin Nutr. 2008; 87(4):1080s-1086s.
66. Hootman J M, Dick R, Agel J. Epidemiology of collegiate injuries for 15 sports: summary and recommendations for injury prevention initiatives. J Athl Train. 2007; 42(2):311-319.
67. Ip T S, Fu S C, Ong M T, Yung P S. Vitamin D deficiency in athletes: Laboratory, clinical and field integration. Asia Pac J Sports Med Arthrosc Rehabil Technol. 2022; 29:22-29.
68. Ivell R, Hunt N, Khan-Dawood F, Dawood M Y. Expression of the human relaxin gene in the corpus luteum of the menstrual cycle and in the prostate. Mol Cell Endocrinol. 1989; 66(2):251-255.
69. Ivell R, Kotula-Balak M, Glynn D, Heng K, Anand-Ivell R. Relaxin family peptides in the male reproductive system—a critical appraisal. Mol Hum Reprod. 2011; 17(2):71-84.
70. Jacobs C A, Hunt E R, Conley C E, et al. Dysregulated Inflammatory Response Related to Cartilage Degradation after ACL Injury. Med Sci Sports Exerc. 2020; 52(3):535-541.
71. Javadian Y, Adabi M, Heidari B, et al. Quadriceps Muscle Strength Correlates With Serum Vitamin D and Knee Pain in Knee Osteoarthritis. Clin JPain. 2017; 33(1):67-70.
72. Kadi R, Milants A, Shahabpour M. Shoulder Anatomy and Normal Variants. J Belg Soc Radiol. 2017; 101(Suppl 2):3.
73. Kennel K A, Drake M T, Hurley D L. Vitamin D deficiency in adults: when to test and how to treat. Mayo Clin Proc. 2010; 85(8):752-757; quiz 757-758.
74. Kim J H, Lee S K, Lee S K, Kim J H, Fredericson M. Relaxin Receptor RXFP1 and RXFP2 Expression in Ligament, Tendon, and Shoulder Joint Capsule of Rats. J Korean Med Sci. 2016; 31(6):983-988.
75. Koay E S, Too C K, Greenwood F C, Bryant-Greenwood G D. Relaxin stimulates collagenase and plasminogen activator secretion by dispersed human amnion and chorion cells in vitro. J Clin Endocrinol Metab. 1983; 56(6):1332-1334.
76. Konopka J A, DeBaun M R, Chang W, Dragoo J L. The Intracellular Effect of Relaxin on Female Anterior Cruciate Ligament Cells. Am J Sports Med. 2016; 44(9):2384-2392.
77. Konopka J A, Hsue L J, Dragoo J L. Effect of Oral Contraceptives on Soft Tissue Injury Risk, Soft Tissue Laxity, and Muscle Strength: A Systematic Review of the Literature. Orthop J Sports Med. 2019; 7(3):2325967119831061.
78. Ksa̧żiek A, Zagrodna A, Slowińska-Lisowska M. Vitamin D, Skeletal Muscle Function and Athletic Performance in Athletes-A Narrative Review. Nutrients. 2019; 11(8).
79. Kuhn K, D'Lima D D, Hashimoto S, Lotz M. Cell death in cartilage. Osteoarthritis Cartilage. 2004; 12(1):1-16.
80. Kyritsis P, Bahr R, Landreau P, Miladi R, Witvrouw E. Likelihood of ACL graft rupture: not meeting six clinical discharge criteria before return to sport is associated with a four times greater risk of rupture. Br J Sports Med. 2016; 50(15):946-951.
81. Lefevre N, Bohu Y, Klouche S, Lecocq J, Herman S. Anterior cruciate ligament tear during the menstrual cycle in female recreational skiers. Orthop Traumatol Surg Res. 2013; 99(5):571-575.
82. Li S, Niu G, Dong X N, et al. Vitamin D Inhibits Activities of Metalloproteinase-9/-13 in Articular Cartilage In Vivo and In Vitro. J Nutr Sci Vitaminol (Tokyo). 2019; 65(2):107-112.
83. Li Z, Yuan B, Pei Z, et al. Circ_0136474 and MMP-13 suppressed cell proliferation by competitive binding to miR-127-5p in osteoarthritis. J Cell Mol Med. 2019; 23(10):6554-6564.
84. Lyman S, Koulouvaris P, Sherman S, et al. Epidemiology of anterior cruciate ligament reconstruction: trends, readmissions, and subsequent knee surgery. J Bone Joint Surg Am. 2009; 91(10):2321-2328.
85. MacLennan A H, Nicolson R, Green R C, Bath M. Serum relaxin and pelvic pain of pregnancy. Lancet. 1986; 2(8501):243-245.
86. Maier G S, Jakobs P, Roth K E, Kurth A A, Maus U. Is there an epidemic vitamin D deficiency in German orthopaedic patients? Clin Orthop Relat Res. 2013; 471(9):3029-3035.
87. Mendias C L, Tatsumi R, Allen R E. Role of cyclooxygenase-1 and -2 in satellite cell proliferation, differentiation, and fusion. Muscle Nerve. 2004; 30(4):497-500.
88. Moon A S, Boudreau S, Mussell E, et al. Current concepts in vitamin D and orthopaedic surgery. Orthop Traumatol Surg Res. 2019; 105(2):375-382.
89. Mu X, Urso M L, Murray K, Fu F, Li Y. Relaxin regulates MMP expression and promotes satellite cell mobilization during muscle healing in both young and aged mice. Am J Pathol. 2010; 177(5):2399-2410.
90. Murray M M, Martin S D, Martin T L, Spector M. Histological changes in the human anterior cruciate ligament after rupture. J Bone Joint Surg Am. 2000; 82(10):1387-1397.
91. Mutsuzaki H, Sakane M, Ikeda K, et al. Histological changes and apoptosis of cartilage layer in human anterior cruciate ligament tibial insertion after rupture. Knee Surg Sports Traumatol Arthrosc. 2007; 15(5):602-609.
92. Myklebust G, Maehlum S, Holm I, Bahr R. A prospective cohort study of anterior cruciate ligament injuries in elite Norwegian team handball. Scand J Med Sci Sports. 1998; 8(3):149-153.
93. Nair V B, Samuel C S, Separovic F, Hossain M A, Wade J D. Human relaxin-2: historical perspectives and role in cancer biology. Amino Acids. 2012; 43(3):1131-1140.
94. Nakagawa Y, Lebaschi A H, Wada S, et al. Duration of postoperative immobilization affects MMP activity at the healing graft-bone interface: Evaluation in a mouse ACL reconstruction model. Journal of Orthopaedic Research. 2019; 37(2):325-334.
95. Neal S, Sykes J, Rigby M, Hess B. A review and clinical summary of vitamin D in regard to bone health and athletic performance. Phys Sportsmed. 2015; 43(2):161-168.
96. Nessler T, Denney L, Sampley J. ACL Injury Prevention: What Does Research Tell Us? Curr Rev Musculoskelet Med. 2017; 10(3):281-288.
97. Nistri S, Bigazzi M, Bani D. Relaxin as a cardiovascular hormone: physiology, pathophysiology and therapeutic promises. Cardiovasc Hematol Agents Med Chem. 2007; 5(2):101-108.
98. Nose-Ogura S, Yoshino O, Yamada-Nomoto K, et al. Oral contraceptive therapy reduces serum relaxin-2 in elite female athletes. J Obstet Gynaecol Res. 2017; 43(3):530-535.
99. Otani S, Kanamoto T, Oyama S, et al. Meniscus surface texture is associated with degenerative changes in biological and biomechanical properties. Scientific Reports. 2022; 12(1):11977.
100. Owens B D, Cameron K L, Clifton K B, Svoboda S J, Wolf J M. Association Between Serum Relaxin and Subsequent Shoulder Instability. Orthopedics. 2016; 39(4):e724-728.
101. Pal C P, Kumar H, Kumar D, et al. Prevalence of vitamin D deficiency in orthopaedic patients—A single centre study. J Clin Orthop Trauma. 2016; 7(Suppl 2):143-146.
102. Palmieri-Smith R M, Brown S R, Wojtys E M, Krishnan C. Functional Resistance Training Improves Thigh Muscle Strength after ACL Reconstruction: A Randomized Clinical Trial. Med Sci Sports Exerc. 2022; 54(10):1729-1737.
103. Papathanasiou I, Michalitsis S, Hantes M E, et al. Molecular changes indicative of cartilage degeneration and osteoarthritis development in patients with anterior cruciate ligament injury. BMC Musculoskelet Disord. 2016; 17:21.
104. Parstorfer M, Profit F, Weiberg N, et al. Increased satellite cell apoptosis in vastus lateralis muscle after anterior cruciate ligament reconstruction. J Rehabil Med. 2021; 53(2):jrm00153.
105. Patton C M, Powell A P, Patel A A. Vitamin D in orthopaedics. J Am Acad Orthop Surg. 2012; 20(3):123-129.
106. Pearson S J, Burgess K E, Onambele G L. Serum relaxin levels affect the in vivo properties of some but not all tendons in normally menstruating young women. Exp Physiol. 2011; 96(7):681-688.
107. Pehrsson M, Westberg L, Landen M, Ekman A. Stable serum levels of relaxin throughout the menstrual cycle: a preliminary comparison of women with premenstrual dysphoria and controls. Arch Womens Ment Health. 2007; 10(4):147-153.
108. Pfeifer M, Begerow B, Minne H W. Vitamin D and muscle function. Osteoporos Int. 2002; 13(3):187-194.
109. Pittayapruek P, Meephansan J, Prapapan O, Komine M, Ohtsuki M. Role of Matrix Metalloproteinases in Photoaging and Photocarcinogenesis. Int J Mol Sci. 2016; 17(6).
110. Pollard C D, Sigward S M, Powers C M. ACL Injury Prevention Training Results in Modification of Hip and Knee Mechanics During a Drop-Landing Task. Orthop J Sports Med. 2017; 5(9):2325967117726267.
111. Powers C M. The Influence of Abnormal Hip Mechanics on Knee Injury: A Biomechanical Perspective. Journal of Orthopaedic & Sports Physical Therapy. 2010; 40(2):42-51.
112. Prodromos C C, Han Y, Rogowski J, Joyce B, Shi K. A meta-analysis of the incidence of anterior cruciate ligament tears as a function of gender, sport, and a knee injury-reduction regimen. Arthroscopy. 2007; 23(12):1320-1325 e1326.
113. Qin X, Garibay-Tupas J, Chua P K, Cachola L, Bryant-Greenwood G D. An autocrine/paracrine role of human decidual relaxin. I. Interstitial collagenase (matrix metalloproteinase-1) and tissue plasminogen activator. Biol Reprod. 1997; 56(4):800-811.
114. Quatman C E, Hewett T E. The anterior cruciate ligament injury controversy: is “valgus collapse” a sex-specific mechanism? Br J Sports Med. 2009; 43(5):328-335.
115. Rahr-Wagner L, Thillemann T M, Mehnert F, Pedersen A B, Lind M. Is the use of oral contraceptives associated with operatively treated anterior cruciate ligament injury? A case-control study from the Danish Knee Ligament Reconstruction Registry. Am J Sports Med. 2014; 42(12):2897-2905.
116. Rai V, Dietz N E, Dilisio M F, Radwan M M, Agrawal D K. Vitamin D attenuates inflammation, fatty infiltration, and cartilage loss in the knee of hyperlipidemic microswine. Arthritis Res Ther. 2016; 18(1):203.
117. Robertson C M, Chen C T, Shindle M K, et al. Failed healing of rotator cuff repair correlates with altered collagenase and gelatinase in supraspinatus and subscapularis tendons. Am J Sports Med. 2012; 40(9):1993-2001.
118. Ruedl G, Ploner P, Linortner I, et al. Are oral contraceptive use and menstrual cycle phase related to anterior cruciate ligament injury risk in female recreational skiers? Knee Surg Sports Traumatol Arthrosc. 2009; 17(9):1065-1069.
119. Sanders T L, Maradit Kremers H, Bryan A J, et al. Incidence of Anterior Cruciate Ligament Tears and Reconstruction: A 21-Year Population-Based Study. Am J Sports Med. 2016; 44(6):1502-1507.
120. Sanders T L, Pareek A, Hewett T E, et al. Long-term rate of graft failure after ACL reconstruction: a geographic population cohort analysis. Knee Surgery, Sports Traumatology, Arthroscopy. 2017; 25(1):222-228.
121. Santora K, Rasa C, Visco D, Steinetz B, Bagnell C. Effects of relaxin in a model of rat adjuvant-induced arthritis. Ann NY Acad Sci. 2005; 1041:481-485.
122. Sarwar M, Du X J, Dschietzig T B, Summers R J. The actions of relaxin on the human cardiovascular system. Br J Pharmacol. 2017; 174(10):933-949.
123. Shelbourne K D, Davis T J, Klootwyk T E. The relationship between intercondylar notch width of the femur and the incidence of anterior cruciate ligament tears. A prospective study. Am J Sports Med. 1998; 26(3):402-408.
124. Sherwood O D. Relaxin's physiological roles and other diverse actions. Endocr Rev. 2004; 25(2):205-234.
125. Sherwood O D, Downing S J, Guico-Lamm M L, et al. The physiological effects of relaxin during pregnancy: studies in rats and pigs. Oxf Rev Reprod Biol. 1993; 15:143-189.
126. Shin C S, Chaudhari A M, Andriacchi T P. The effect of isolated valgus moments on ACL strain during single-leg landing: a simulation study. J Biomech. 2009; 42(3):280-285.
127. Shin C S, Chaudhari A M, Andriacchi T P. Valgus plus internal rotation moments increase anterior cruciate ligament strain more than either alone. Med Sci Sports Exerc. 2011; 43(8):1484-1491.
128. Singh R, Letai A, Sarosiek K. Regulation of apoptosis in health and disease: the balancing act of BCL-2 family proteins. Nat Rev Mol Cell Biol. 2019; 20(3):175-193.
129. Skinner S J, Campos G A, Liggins G C. Collagen content of human amniotic membranes: effect of gestation length and premature rupture. Obstet Gynecol. 1981; 57(4):487-489.
130. Spindler K P, Wright R W. Anterior Cruciate Ligament Tear. New England Journal of Medicine. 2008; 359(20):2135-2142.
131. Stockton K A, Mengersen K, Paratz J D, Kandiah D, Bennell K L. Effect of vitamin D supplementation on muscle strength: a systematic review and meta-analysis. Osteoporos Int. 2011; 22(3):859-871.
132. Teerlink J R, Cotter G, Davison B A, et al. Serelaxin, recombinant human relaxin-2, for treatment of acute heart failure (RELAX-AHF): a randomised, placebo-controlled trial. Lancet. 2013; 381(9860):29-39.
133. Timms P M, Mannan N, Hitman G A, et al. Circulating MMP9, vitamin D and variation in the TIMP-1 response with VDR genotype: mechanisms for inflammatory damage in chronic disorders? Qjm. 2002; 95(12):787-796.
134. Tran E Y, Uhl R L, Rosenbaum A J. Vitamin D in Orthopaedics. JBJS Rev. 2017; 5(8):e1.
135. Vinall R L, Mahaffey C M, Davis R R, et al. Dual blockade of PKA and NF-kappaB inhibits H2 relaxin-mediated castrate-resistant growth of prostate cancer sublines and induces apoptosis. Horm Cancer. 2011; 2(4):224-238.
136. Wang X, Cicuttini F, Jin X, et al. Knee effusion-synovitis volume measurement and effects of vitamin D supplementation in patients with knee osteoarthritis. Osteoarthritis Cartilage. 2017; 25(8):1304-1312.
137. Wilkinson T N, Speed T P, Tregear G W, Bathgate R A. Evolution of the relaxin-like peptide family. BMC Evol Biol. 2005; 5:14.
138. Wiqvist I, Norstrom A, O'Byrne E, Wiqvist N. Regulatory influence of relaxin on human cervical and uterine connective tissue. Acta Endocrinol (Copenh). 1984; 106(1):127-132.
139. Wojtys E M, Huston L J, Boynton M D, Spindler K P, Lindenfeld T N. The effect of the menstrual cycle on anterior cruciate ligament injuries in women as determined by hormone levels. Am J Sports Med. 2002; 30(2):182-188.
140. Wolf J M, Cameron K L, Clifton K B, Owens B D. Serum relaxin levels in young athletic men are comparable with those in women. Orthopedics. 2013; 36(2):128-131.
141. Wolf J M, Scher D L, Etchill E W, et al. Relationship of relaxin hormone and thumb carpometacarpal joint arthritis. Clin Orthop Relat Res. 2014; 472(4):1130-1137.
142. Wood M L, Luthin W N, Lester G E, Dahners L E. Tendon creep is potentiated by NKISK and relaxin which produce collagen fiber sliding. Iowa Orthop J 2003; 23:75-79.
143. Wreje U, Kristiansson P, Aberg H, Bystrom B, von Schoultz B. Serum levels of relaxin during the menstrual cycle and oral contraceptive use. Gynecol Obstet Invest. 1995; 39(3):197-200.
144. Yue L, DeFroda S F, Sullivan K, Garcia D, Owens B D. Mechanisms of Bone Tunnel Enlargement Following Anterior Cruciate Ligament Reconstruction. JBJS Rev. 2020; 8(4):e0120.
145. Zhao D, Pan J-k, Lin F-z, et al. Risk Factors for Revision or Rerupture After Anterior Cruciate Ligament Reconstruction: A Systematic Review and Meta-analysis. The American Journal of Sports Medicine. 2023; 51(11):3053-3075.
146. Zhao S, Zhao Y, Guo J, et al. Downregulation of MMP1 in MDS-derived mesenchymal stromal cells reduces the capacity to restrict MDS cell proliferation. Sci Rep. 2017; 7:43849.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following claims.

What is claimed is:

1. A method for preventing and/or treating a connective tissue injury characteristic, the method comprising the steps of:
(1) obtaining a subject at risk of a connective tissue injury or suspected of being at risk; and
(2) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury;
wherein the subject is a subject that has previously undergone a surgical reconstruction, and/or a normal healthy subject, lacking a pre-diagnosed connective tissue disease or condition, and step (2) comprises in part a prophylactic method used to prevent a connective tissue injury and/or rupture.

2. The method of claim 1, wherein the connective tissue comprises a ligament, and the antagonist comprises folic acid, NADH, or a combination thereof.

3. The method of claim 1, wherein the subject is expected to or likely to participate in an activity, a sport, or a movement in the future that can increase the chances of a connective tissue injury occurring in the subject; or wherein the subject is at a risk of or in need of a connective tissue care, having a diagnosed connective tissue injury characteristic, or has been determined to be at a risk of a connective tissue injury by a health care provider; or wherein the subject is suspected of having, or has been diagnosed by a health care provider as having, a disorder including an autoimmune disease of connective tissue, an undifferentiated connective tissue disorder, a myxomatous degeneration, a congenital disease, a neoplasm, or a combination thereof.

4. The method of claim 1, wherein the antagonist further comprises a therapeutic agent, a supplement, a formulation, a natural product, vitamin D, a drug, a biologic, an aerosol, a liquid, a particle, microparticle, or nanoparticle, a biologic, or a combination thereof.

5. The method of claim 1, wherein the antagonist includes a daily dose of about 400 micrograms/day antagonist for a healthy human subject.

6. A method for cultivating a connective tissue in vitro, the method comprising the step of contacting the culturing connective tissue with an antagonist of relaxin-2 and/or an antagonist of a receptor of relaxin-2, or a precursor of said antagonist, before, during, or after said cultivating, whereby one or more connective tissue characteristics are modulated/changed.

7. The method of claim 6, further comprising wherein the connective tissue is suitable for implantation into a subject in need thereof; wherein the connective tissue is a cell generated tissue graft; wherein the connective tissue cultivated in vitro is derived from or originates from a subject undergoing, or planned to undergo, a ligament reconstruction surgery; or a combination thereof.

8. The method of claim 6, wherein the connective tissue includes ligament, tendon, cartilage, intervertebral disc, cornea, or a combination thereof; and/or wherein the connective tissue comprises a fibrillar collagen or a collagen.

9. The method of claim 6, wherein the connective tissue comprises collagen and wherein the collagen comprises type I (or IA), II, III (or IIIA), V, XI, or a combination thereof.

10. The method of claim 1, wherein the antagonist is a blocker, an orthosteric antagonist, allosteric antagonist, conformation changing binder, a modulator, a partial inverse agonist/inverse agonist, or a combination thereof.

11. The method of claim 1, wherein the antagonist comprises a relaxin antagonist/relaxin receptor antagonist from Table 4 below, or from a library of known therapeutic agents:
TABLE 4
GENERIC_NAME:
Abarelix,
Acarbose,
Adenosine phosphate,
Amphetamine,
Anagrelide,
Argatroban,
Betazole,
Biotin,
Bleomycin,
Calcium acetate,
Calcium glucoheptonate,
Capreomycin,
Carboprost tromethamine,
Cefotiam,
Cefpiramide,
Ceruletide,
Cetrorelix,
Chlorthalidone,
Chlorzoxazone,
Cidofovir,
Deferiprone,
Desmopressin,
Dicoumarol,
Digoxin,
Droperidol,
Edotreotide gallium Ga-68,
Entecavir,
Ertapenem,
Flavone,
Folic acid,
Glutamic acid,
Glutathione,
Goserelin,
Hyaluronic acid,
Indecainide,
Indium In-111 pentetreotide,
Iomeprol,
Isoetharine,
Leuprolide,
Lipoic acid,
Lorazepam,
Mangafodipir,
Mefloquine,
Mesalazine,
Methocarbamol,
Methysergide,
Midodrine,
Miglustat,
Milrinone,
Minoxidil,
Mupirocin,
NADH,
Nelfinavir,
Nitisinone,
Nonoxynol-9,
Norepinephrine,
Octreotide,
Palonosetron,
Pamidronic acid,
Penciclovir,
Pentagastrin,
Phenoxymethylpenicillin,
Phentermine,
Phenylbutyric acid,
Phenylephrine,
Phenylpropanolamine,
Plicamycin,
Polymyxin B,
Pralatrexate,
Protokylol,
Protriptyline,
Pyrazinamide,
Pyrithione,
Raltegravir,
Raltitrexed,
Salicylamide,
Sapropterin,
Succinic acid,
Tenapanor,
Tezacaftor,
Theophylline,
Tiopronin,
Tranexamic acid,
Travoprost,
Treprostinil,
Trihexyphenidyl.
Troglitazone,
Tryptophan,
Unoprostone,
Valrubicin, and
Zoledronic acid.

12. A method for preventing and/or treating a connective tissue injury characteristic, the method comprising the steps of:
(1) providing or obtaining a subject; and
(2) administering vitamin D (D₁, D₂, D₃, D₄, D₅, or an analogue thereof) to the subject, or a precursor of said vitamin D, whereby one or more connective tissue characteristics are modulated/changed in the subject such as to prevent and/or treat the connective tissue injury.

13. The method of claim 12, further comprising the step of:
(3) administering an antagonist of relaxin-2 and/or an antagonist of a receptor of relaxin-2 to the subject, or a precursor of said antagonist, whereby one or more connective tissue characteristics are modulated/changed in the subject such as to prevent and/or treat the connective tissue injury.

14. The method of claim 12, wherein the vitamin D comprises vitamin D₃ (cholecalciferol), vitamin D₂ (ergocalciferol), or a combination thereof.

15. The method of claim 12, wherein the analogue comprises Alfacalcidol, Calcipotriol (calcipotriene), Doxercalciferol, Falecalcitriol, Paricalcitol, Tacalcitol, or a combination thereof.

16. The method of claim 12, wherein a Type I collagen expression is observably increased after vitamin D in female cells compared to such observable expression under the same conditions in male cells.

17. The method of claim 1, wherein an expression/activity of MMP1 and/or MMP13 is decreased by the method.

18. The method of claim 12, wherein an expression/activity of MMP1 and/or MMP13 is decreased by the method.

19. The method of claim 12, wherein an MMP13 gene expression is reduced after one or more vitamin D treatments at about 10 nmol/L in female cells and at about 100 nmol/L in male cells.
